(19) United States
(12) Patent Application Publication
(10) Pub. No.: US 2014/0012056 A1
(43) Pub. Date: Jan. 9, 2014

(54) METHOD FOR PRODUCING 1-HEXENE

(71) Applicants: Takayuki Hishiya, Ichihara (JP); Takahiro Hino, Ichihara (JP); Taichi Senda, Ichihara (JP)

(72) Inventors: Takayuki Hishiya, Ichihara (JP); Takahiro Hino, Ichihara (JP); Taichi Senda, Ichihara (JP)

(73) Assignee: SUMITOMO CHEMICAL COMPANY, LIMITED, Chuo-ku, Tokyo (JP)

(21) Appl. No.: 14/007,715

(22) PCT Filed: Mar. 29, 2012
(86) PCT No.: PCT/JP2012/059296

§ 371 Date: Sep. 26, 2013

(30) Foreign Application Priority Data
Mar. 30, 2011 (JP) 2011-074577
Apr. 6, 2011 (JP) 2011-084371

(51) Int. Cl. C07C 2/30

(52) U.S. Cl. CPC C07C 2/30
USPC 585/511

(57) ABSTRACT

Disclosed is a method for producing 1-hexene that is capable of reducing the amount of by-product polymers when 1-hexene is produced through the trimerization reaction of ethylene. The method for producing 1-hexene comprises the following steps 1 and 2:
step 1: the step of preparing a catalytic component by bringing a transition metal complex represented by any one of formulae (1-1) to (1-3) into contact with a specific organic aluminum compound in the absence of ethylene; and
step 2: the step of trimerizing ethylene in the presence of a catalyst obtainable by bringing the catalytic component obtained in step 1 into contact with a specific boron compound.

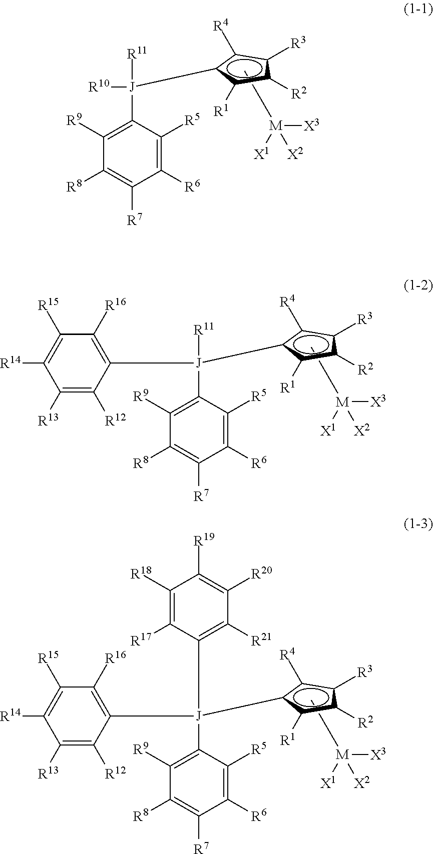

### TECHNICAL FIELD

The present invention relates to a method for producing 1-hexene.

### BACKGROUND ART

α-olefin is an industrially important monomer raw material that is produced by the oligomerization of ethylene using a metal catalyst. However, the oligomerization of ethylene usually gives α-olefin mixtures according to Schulz-Flory distribution. Therefore, the development of a catalyst system capable of selectively producing one species of α-olefin is very important industrially.

For example, Patent Literature 1 has reported that a half-metallocene titanium complex represented by the formula (Cp-B(R)ₙAr)TiR¹₃ functions as a catalytic component for selective trimerization of ethylene in the presence of an activating co-catalytic component.

Among these catalysts for selective ethylene trimerization, a half-metallocene titanium complex (carbon-bridged Cp-Ar complex) wherein cyclopentadiene is bonded to a substituted aryl group via a carbon atom, such as [1-(1-methyl-1-(3,5-dimethylphenyl)ethyl)-3-trimethylsilyl-cyclopentadienyl]titanium trichloride, has been reported to function as an efficient catalyst for ethylene trimerization under the condition of 30° C. with MAO (methylaluminoxane) as an activating co-catalytic component (see e.g., Non-Patent Literature 1). On the other hand, [dimethylphenylsilylcyclopentadienyl]titanium trichloride, which is a half-metallocene titanium complex (silicon-bridged Cp-Ar complex) wherein cyclopentadiene is bonded to a substituted aryl group via a silicon atom, has been reported to have low catalytic activity in ethylene trimerization reaction under the same conditions as above and to yield a larger amount of by-product polymers (see Non-Patent Literature1).

Moreover, it has been reported that a catalyst system for ethylene trimerization using a carbon-bridged Cp-Ar complex and MAO exhibits much lower catalytic activity in 1-hexene production under the high temperature condition of 80° C. than that of 30° C. and yields a larger amount of by-product polymers under the high temperature conditions of 80° C. than that of 30° C. (see Non-Patent Literature2).

It has been reported that in ethylene trimerization reaction at 30° C. using such a carbon-bridged Cp-Ar complex, the amount of by-product polymers can be reduced by bringing, for example, [1-(1-methyl-1-(3,5-dimethylphenyl)ethyl)-3-trimethylsilyl-cyclopentadienyl]titanium trichloride into contact with a toluene solution containing MAO, followed by ethylene trimerization reaction (see Non-Patent Literature3).

### CITATION LIST

### Patent Literature

PATENT LITERATURE 1: JP-A-2004-524959

### Non-Patent Literature

NON-PATENT LITERATURE 1: Organometallics 2002, 21, 5122-5135.
NON-PATENT LITERATURE 2: Chinese Journal of Chemistry 2006, 24, 1397-1401.
NON-PATENT LITERATURE 3: J. Mol. Catal. A: Chem. 2006, 248, 237-247.

### SUMMARY OF INVENTION

### Technical Problem

Under such circumstances, an object of the present invention is to provide a method for producing 1-hexene that is capable of reducing the amount of by-product polymers when 1-hexene is produced through the trimerization reaction of ethylene.

### Solution to Problem

Specifically, a 1st aspect of the present invention relates to a method for producing 1-hexene, comprising the following steps 1 and 2:

step 1: the step of preparing a catalytic component by bringing a transition metal complex represented by any one of formulae (1-1) to (1-3) into contact with the following compound (A) in the absence of ethylene; and

step 2: the step of trimerizing ethylene in the presence of a catalyst obtainable by bringing the catalytic component obtained in step 1 into contact with the following compound (B).

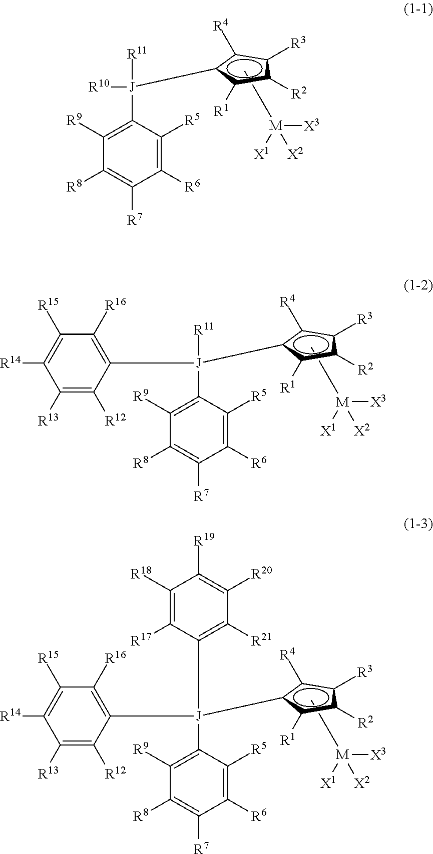

wherein

M represents a transition metal atom of Group 4 of the Periodic Table of the Elements; J represents a carbon atom or a silicon atom;

R¹, R², R³, R⁴, R⁵, R⁶, R⁷, R⁸, R⁹, R¹², R¹³, R¹⁴, R¹⁵, R¹⁶, R¹⁷, R¹⁸, R¹⁹, R²⁰, R²¹, X¹, X² and X³ each independently represent
a hydrogen atom, a halogen atom,
an alkyl group having 1 to 20 carbon atoms which may have a halogen atom as a substituent,
an alkoxy group having 1 to 20 carbon atoms which may have a halogen atom as a substituent,
an aryl group having 6 to 20 carbon atoms which may have a halogen atom as a substituent,
an aryloxy group having 6 to 20 carbon atoms which may have a halogen atom as a substituent,
an aralkyl group having 7 to 20 carbon atoms which may have a halogen atom as a substituent,
an aralkyloxy group having 7 to 20 carbon atoms which may have a halogen atom as a substituent,
a substituted silyl group represented by —Si(R²²)₃, wherein the three R²² groups each independently represent a hydrogen atom, a hydrocarbyl group or a halogenated hydrocarbyl group, and the total number of the carbon atoms in the three R²² groups is 1 to 20, or
a disubstituted amino group represented by —N(R²³)₂, wherein the two R²³ groups each independently represent a hydrocarbyl group or a halogenated hydrocarbyl group, and the total number of the carbon atoms in the two R²³ groups is 2 to 20, and
R¹⁰ and R¹¹ each independently represent
a hydrogen atom, a halogen atom,
an alkyl group having 1 to 20 carbon atoms which may have a halogen atom as a substituent,
an alkoxy group having 1 to 20 carbon atoms which may have a halogen atom as a substituent,
an aryloxy group having 6 to 20 carbon atoms which may have a halogen atom as a substituent,
an aralkyl group having 7 to 20 carbon atoms which may have a halogen atom as a substituent,
an aralkyloxy group having 7 to 20 carbon atoms which may have a halogen atom as a substituent,
a substituted silyl group represented by —Si(R²²)₃, wherein the three R²² groups each independently represent a hydrogen atom, a hydrocarbyl group or a halogenated hydrocarbyl group, and the total number of the carbon atoms in the three R²² groups is 1 to 20, or
a disubstituted amino group represented by —N(R²³)₂, wherein the two R²³ groups each independently represent a hydrocarbyl group or a halogenated hydrocarbyl group, and the total number of the carbon atoms in the two R²³ groups is 2 to 20;
of R¹, R², R³ and R⁴, two group bonded to two adjoining carbon atoms may be bonded to each other to form a ring together with the two carbon atoms to which the two groups are bonded, of R⁵, R⁶, R⁷, R⁸ and R⁹, two group bonded to two adjoining carbon atoms may be bonded to each other to form a ring together with the two carbon atoms to which the two groups are bonded, of R¹², R¹³, R¹⁴, R¹⁵ and R¹⁶, two group bonded to two adjoining carbon atoms may be bonded to each other to form a ring together with the two carbon atoms to which the two groups are bonded, of R¹⁷, R¹⁸, R¹⁹, R²⁰ and R²¹, two group bonded to two adjoining carbon atoms may be bonded to each other to form a ring together with the two carbon atoms to which the two groups are bonded, and R¹⁰ and R¹¹ may be bonded to each other to form a ring together with J to which they are bonded.

compound (A): an organic aluminum compound represented by formula

(E¹)ₐAl(G)₃₋ₐ,

wherein E¹ represents a hydrocarbyl group having 2 to 8 carbon atoms; G represents a hydrogen atom or a halogen atom; a represents an integer of 1 to 3; in the case where more than one E¹ groups exist, the E¹ groups may be the same as or different from each other; and in the case where more than one G groups exist, the G groups may be the same as or different from each other, and

compound (B): one or more boron compounds selected from the compound group consisting of the following compounds (B1), (B2) and (B3):

(B1): a boron compound represented by formula BQ¹Q²Q³,

(B2): a borate compound represented by formula T⁺(BQ⁴Q⁵Q⁶Q⁷)⁻, and

(B3): a borate compound represented by formula (L-H)⁺(BQ⁸Q⁹Q¹⁰Q¹¹)⁻, wherein

B represents a trivalent boron; Q¹, Q², Q³, Q⁴, Q⁵, Q⁶, Q⁷, Q⁸, Q⁹, Q¹⁰ and Q¹¹ are the same as or different from each other and each represent a halogen atom, a hydrocarbyl group having 1 to 20 carbon atoms which may have a halogen as a substituent, a hydrocarbylsilyl group having 1 to 20 carbon atoms which may have a halogen as a substituent, an alkoxy group having 1 to 20 carbon atoms which may have a halogen as a substituent or a dihydrocarbylamino group having 2 to 20 carbon atoms which may have a halogen as a substituent; T⁺ represents an inorganic or organic cation; and (L-H)⁺ represents a Broensted acid.

### Advantageous Effects of Invention

The present invention can provide a method for producing 1-hexene that is capable of reducing the amount of by-product polymers even under high temperature conditions when 1-hexene is produced through the trimerization reaction of ethylene.

### DESCRIPTION OF EMBODIMENTS

### <Method for Producing 1-Hexene>

A method for producing 1-hexene of the present invention is a method for producing 1-hexene from ethylene and is a production method comprising the following steps 1 and 2:

step 1: the step of preparing a catalytic component by bringing a transition metal complex represented by any one of formulae (1-1) to (1.3) into contact with a compound (A) in the absence of ethylene; and

step 2: the step of trimerizing ethylene in the presence of a catalyst obtainable by bringing the catalytic component obtained in step 1 into contact with a compound (B).

### (Step 1)

The transition metal complex represented by any one of formulae (1-1) to (1-3) (hereinafter, also abbreviated to a “transition metal complex (1-1)”, etc.) used in the step 1 will be described in detail.

In the transition metal complexes (1-1) to (1-3), M represents an element of Group 4 of the Periodic Table of the Elements, and examples thereof include titanium, zirconium and hafnium atoms. Among them, a titanium atom is preferable.

In the transition metal complexes (1-1) to (1-3), J represents a carbon atom or a silicon atom and is preferably a silicon atom.

In the transition metal complexes (1-1) to (1-3), the substituents R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, X¹, X² and X³ are as defined above, and examples thereof are shown below.

The halogen atom is a fluorine, chlorine, bromine or iodine atom and is preferably a chlorine atom.

Examples of the “alkyl group having 1 to 20 carbon atoms” in the alkyl group having 1 to 20 carbon atoms which may have a halogen atom as a substituent include methyl, ethyl, n-propyl, isopropyl, n-butyl, sec-butyl, tert-butyl, n-pentyl, neopentyl, amyl, n-hexyl, heptyl, n-octyl, n-nonyl, n-decyl, n-dodecyl, n-tridecyl, tetradecyl, pentadecyl, hexadecyl, heptadecyl, octadecyl, nonadecyl and n-eicosyl groups. Of them, a preferable alkyl group is an alkyl group having 1 to 10 carbon atoms, and more preferable examples thereof can include methyl, ethyl, isopropyl, tert-butyl and amyl groups. Moreover, the phrase “may have a halogen atom as a substituent” in the “alkyl group which may have a halogen atom as a substituent” means that a part or all of the hydrogen atoms in the alkyl group may be substituted by a halogen atom. Examples of the halogen atom are as described above. When the alkyl group has a halogen atom as a substituent, the number of its carbon atoms is preferably in the range of 1 to 20, more preferably in the range of 1 to 10. Preferable examples of the alkyl group having a halogen atom as a substituent can include fluoromethyl, difluoromethyl, trifluoromethyl, chloromethyl, dichloromethyl, trichloromethyl, bromomethyl, dibromomethyl, tribromomethyl, fluoroethyl, perfluoropropyl, perfluorobutyl, perfluoropentyl and perfluorohexyl groups.

Examples of the “aryl group having 6 to 20 carbon atoms” in the aryl group having 6 to 20 carbon atoms which may have a halogen atom as a substituent include phenyl, 2-tolyl, 3-tolyl, 4-tolyl, 2,3-xylyl, 2,4-xylyl, 2,5-xylyl, 2,6-xylyl, 3,4-xylyl, 3,5-xylyl, 2,3,4-trimethylphenyl, 2,3,5-trimethylphenyl, 2,3,6-trimethylphenyl, 2,4,6-trimethylphenyl, 3,4,5-trimethylphenyl, 2,3,4,5-tetramethylphenyl, 2,3,4,6-tetramethylphenyl, 2,3,5,6-tetramethylphenyl, pentamethylphenyl, ethylphenyl, n-propylphenyl, isopropylphenyl, n-butylphenyl, sec-butylphenyl, tert-butylphenyl, n-pentylphenyl, neopentylphenyl, n-hexylphenyl, n-octylphenyl, n-decylphenyl, n-dodecylphenyl, n-tetradecylphenyl, naphthyl and anthracenyl groups. Of them, a preferable aryl group is an aryl group having 6 to 10 carbon atoms, and more preferable examples thereof can include a phenyl group. Moreover, the phrase “may have a halogen atom as a substituent” in the “aryl group which may have a halogen atom as a substituent” means that a part or all of the hydrogen atoms in the aryl group may be substituted by a halogen atom. Examples of the halogen atom are as described above. When the aryl group has a halogen atom as a substituent, the number of its carbon atoms is preferably in the range of 6 to 20, more preferably in the range of 6 to 10. Preferable examples of the aryl group having a halogen atom as a substituent can specifically include fluorophenyl, difluorophenyl, trifluorophenyl, tetrafluorophenyl, pentafluorophenyl, chlorophenyl, bromophenyl and iodophenyl groups.

Examples of the “aralkyl group having 7 to 20 carbon atoms” in the aralkyl group having 7 to 20 carbon atoms which may have a halogen atom as a substituent include benzyl, (2-methylphenyl)methyl, (3-methylphenyl)methyl, (4-methylphenyl)methyl, (2,3-dimethylphenyl)methyl, (2,4-dimethylphenyl)methyl, (2,5-dimethylphenyl)methyl, (2,6-dimethylphenyl)methyl, (3,4-dimethylphenyl)methyl, (3,5-dimethylphenyl)methyl, (2,3,4-trimethylphenyl)methyl, (2,3,5-trimethylphenyl)methyl, (2,3,6-trimethylphenyl)methyl, (3,4,5-trimethylphenyl)methyl, (2,4,6-trimethylphenyl)methyl, (2,3,4,5-tetramethylphenyl)methyl, (2,3,4,6-tetramethylphenyl)methyl, (2,3,5,6-tetramethylphenyl)methyl, (pentamethylphenyl)methyl, (ethylphenyl)methyl, (n-propylphenyl)methyl, (isopropylphenyl)methyl, (n-butylphenyl)methyl, (sec-butylphenyl)methyl, (tert-butylphenyl)methyl, (n-pentylphenyl)methyl, (neopentylphenyl)methyl, (n-hexylphenyl)methyl, (n-octylphenyl)methyl, (n-decylphenyl)methyl, (n-dodecylphenyl)methyl, naphthylmethyl and anthracenylmethyl groups. Of them, a preferable aralkyl group is an aralkyl group having 7 to 10 carbon atoms, and more preferable examples thereof can include a benzyl group. Moreover, the phrase “may have a halogen atom as a substituent” in the “aralkyl group which may have a halogen atom as a substituent” means that a part or all of the hydrogen atoms in the aralkyl group may be substituted by a halogen atom. Examples of the halogen atom are as described above. When the aralkyl group has a halogen atom as a substituent, the number of its carbon atoms is preferably in the range of 7 to 20, more preferably in the range of 7 to 10.

Examples of the “alkoxy group having 1 to 20 carbon atoms” in the alkoxy group having 1 to 20 carbon atoms which may have a halogen atom as a substituent include methoxy, ethoxy, n-propoxy, isopropoxy, n-butoxy, sec-butoxy, tert-butoxy, n-pentyloxy, neopentyloxy, n-hexyloxy, n-octyloxy, n-nonyloxy, n-decyloxy, n-undecyloxy, n-dodecyloxy, tridecyloxy, tetradecyloxy, n-pentadecyloxy, hexadecyloxy, heptadecyloxy, octadecyloxy, nonadecyloxy and n-eicosyloxy groups. Of them, a preferable alkoxy group is an alkoxy group having 1 to 10 carbon atoms, and more preferable examples thereof can include methoxy, ethoxy and tert-butoxy groups. Moreover, the phrase “may have a halogen atom as a substituent” in the “alkoxy group which may have a halogen atom as a substituent” means that a part or all of the hydrogen atoms in the alkoxy group may be substituted by a halogen atom. Examples of the halogen atom are as described above. When the alkoxy group has a halogen atom as a substituent, the number of its carbon atoms is preferably in the range of 1 to 20, more preferably in the range of 1 to 10.

Examples of the “alkoxy group having 2 to 20 carbon atoms” in the alkoxy group having 2 to 20 carbon atoms which may have a halogen atom as a substituent include ethoxy, n-propoxy, isopropoxy, n-butoxy, sec-butoxy, tert-butoxy, n-pentyloxy, neopentyloxy, n-hexyloxy, n-octyloxy, n-nonyloxy, n-decyloxy, n-undecyloxy, n-dodecyloxy, tridecyloxy, tetradecyloxy, n-pentadecyloxy, hexadecyloxy, heptadecyloxy, octadecyloxy, nonadecyloxy and n-eicosyloxy groups. Of them, a preferable alkoxy group is an alkoxy group having 2 to 10 carbon atoms, and more preferable examples thereof can include ethoxy and tert-butoxy groups. Moreover, the phrase “may have a halogen atom as a substituent” in the “alkoxy group which may have a halogen atom as a substituent” means that a part or all of the hydrogen atoms in the alkoxy group may be substituted by a halogen atom. Examples of the halogen atom are as described above. When the alkoxy group has a halogen atom as a substituent, the number of its carbon atoms is preferably in the range of 2 to 20, more preferably in the range of 2 to 10.

Examples of the “aryloxy group having 6 to 20 carbon atoms” in the aryloxy group having 6 to 20 carbon atoms which may have a halogen atom as a substituent include phenoxy, 2-methylphenoxy, 3-methylphenoxy, 4-methylphenoxy, 2,3-dimethylphenoxy, 2,4-dimethylphenoxy, 2,5-dimethylphenoxy, 2,6-dimethylphenoxy, 3,4-dimethylphenoxy, 3,5-dimethylphenoxy, 2,3,4-trimethylphenoxy, 2,3,5-trimethylphenoxy, 2,3,6-trimethylphenoxy, 2,4,5-trimethylphenoxy, 2,4,6-trimethylphenoxy, 3,4,5-trimethylphenoxy, 2,3,4,5-tetramethylphenoxy, 2,3,4,6-tetramethylphenoxy, 2,3,5,6-tetramethylphenoxy, pentamethylphenoxy, ethylphenoxy, n-propylphenoxy, isopropylphenoxy, n-butylphenoxy, sec-butylphenoxy, tert-butylphenoxy, n-hexylphenoxy, n-octylphenoxy, n-decylphenoxy, n-tetradecylphenoxy, naphthoxy and anthracenoxy groups. Of them, a preferable aryloxy group is an aryloxy group having 6 to 10 carbon atoms, and more preferable examples thereof can include phenoxy, 2-methylphenoxy, 3-methylphenoxy and 4-methylphenoxy groups. Moreover, the phrase “may have a halogen atom as a substituent” in the “aryloxy group which may have a halogen atom as a substituent” means that a part or all of the hydrogen atoms in the aryloxy group may be substituted by a halogen atom. Examples of the halogen atom are as described above. When the aryloxy group has a halogen atom as a substituent, the number of its carbon atoms is preferably in the range of 6 to 20, more preferably in the range of 6 to 10.

Examples of the “aralkyloxy group having 7 to 20 carbon atoms” in the aralkyloxy group having 7 to 20 carbon atoms which may have a halogen atom as a substituent include benzyloxy, (2-methylphenyl)methoxy, (3-methylphenyl)methoxy, (4-methylphenyl)methoxy, (2,3-dimethylphenyl)methoxy, (2,4-dimethylphenyl)methoxy, (2,5-dimethylphenyl)methoxy, (2,6-dimethylphenyl)methoxy, (3,4-dimethylphenyl)methoxy, (3,5-dimethylphenyl)methoxy, (2,3,4-trimethylphenyl)methoxy, (2,3,5-trimethylphenyl)methoxy, (2,3,6-trimethylphenyl)methoxy, (2,4,5-trimethylphenyl)methoxy, (2,4,6-trimethylphenyl)methoxy, (3,4,5-trimethylphenyl)methoxy, (2,3,4,5-tetramethylphenyl)methoxy, (2,3,4,6-tetramethylphenyl)methoxy, (2,3,5,6-tetramethylphenyl)methoxy, (pentamethylphenyl)methoxy, (ethylphenyl)methoxy, (n-propylphenyl)methoxy, (isopropylphenyl)methoxy, (n-butylphenyl)methoxy, (sec-butylphenyl)methoxy, (tert-butylphenyl)methoxy, (n-hexylphenyl)methoxy, (n-octylphenyl)methoxy, (n-decylphenyl)methoxy, naphthylmethoxy and anthracenylmethoxy groups. Of them, a preferable aralkyloxy group is an aralkyloxy group having 7 to 10 carbon atoms, and more preferable examples thereof can include a benzyloxy group. Moreover, the phrase “may have a halogen atom as a substituent” in the “aralkyloxy group which may have a halogen atom as a substituent” means that a part or all of the hydrogen atoms in the aralkyloxy group may be substituted by a halogen atom. Examples of the halogen atom are as described above. When the aralkyloxy group has a halogen atom as a substituent, the number of its carbon atoms is preferably in the range of 7 to 20, more preferably in the range of 7 to 10.

In the substituted silyl group represented by —Si(R²²)₃, wherein the three R²² groups each independently represent a hydrogen atom, a hydrocarbyl group or a halogenated hydrocarbyl group, and the total number of the carbon atoms in the three R²² groups is 1 to 20, the R²² groups are each independently a hydrogen atom; a hydrocarbyl group such as an alkyl group having 1 to 10 carbon atoms (e.g., methyl, ethyl, n-propyl, isopropyl, n-butyl, sec-butyl, tert-butyl, isobutyl, n-pentyl, n-hexyl, cyclohexyl, n-heptyl, n-octyl, n-nonyl and n-decyl groups) and an aryl group (e.g., a phenyl group); or a halogenated hydrocarbyl group obtained by substituting a part or all of the hydrogen atoms in the hydrocarbyl group with a halogen atom, and the total number of the carbon atoms in the three R²² groups is in the range of 1 to 20. The total number of the carbon atoms in these three R²² groups is preferably in the range of 3 to 18. Examples of the substituted silyl group include: monosubstituted silyl groups having one hydrocarbyl or halogenated hydrocarbyl group, such as methylsilyl, ethylsilyl and phenylsilyl groups, and groups obtained by substituting a part or all of the hydrogen atoms in the hydrocarbyl groups of these groups with a halogen atom; disubstituted silyl groups having two hydrocarbyl and/or halogenated hydrocarbyl groups, such as dimethylsilyl, diethylsilyl and diphenylsilyl groups, and groups obtained by substituting a part or all of the hydrogen atoms in the hydrocarbyl groups of these groups with a halogen atom; and trisubstituted silyl group having three hydrocarbyl and/or halogenated hydrocarbyl groups, such as trimethylsilyl, triethylsilyl, tri-n-propylsilyl, triisopropylsilyl, tri-n-butylsilyl, tri-sec-butylsilyl, tri-tert-butylsilyl, tri-isobutylsilyl, tert-butyl-dimethylsilyl, tri-n-pentylsilyl, tri-n-hexylsilyl, tricyclohexylsilyl and triphenylsilyl groups, and groups obtained by substituting a part or all of the hydrogen atoms in the hydrocarbyl groups of these groups with a halogen atom. Of them, trisubstituted silyl groups are preferable, and trimethylsilyl, tert-butyldimethylsilyl and triphenylsilyl groups, and groups obtained by substituting a part or all of the hydrogen atoms in these groups with a halogen atom are more preferable.

In the disubstituted amino group represented by —N(R²³)₂, wherein the two R²³ groups each independently represent a hydrocarbyl group or a halogenated hydrocarbyl group, and the total number of the carbon atoms in the two R²³ groups is 2 to 20, the R²³ groups each independently represent a hydrocarbyl group or a halogenated hydrocarbyl group, and the total number of the carbon atoms in the two R²³ groups is in the range of 2 to 20, more preferably in the range of 2 to 10. The hydrocarbyl group and the halogenated hydrocarbyl group are the same as those described as a hydrocarbyl group and a halogenated hydrocarbyl group for the substituted silyl group. Moreover, these two R²³ groups may be bonded to each other to form a ring together with the nitrogen atom to which they are bonded. Examples of such a disubstituted amino group include dimethylamino, diethylamino, di-n-propylamino, diisopropylamino, di-n-butylamino, di-sec-butylamino, di-tort-butylamino, di-isobutylamino, tert-butylisopropylamino, di-n-hexylamino, di-n-octylamino, di-n-decylamino, diphenylamino, bistrimethylsilylamino, bis-tert-butyldimethylsilylamino, pyrrolyl, pyrrolidinyl, piperidinyl, carbazolyl, dihydroindolyl and dihydroisoindolyl groups, and groups obtained by substituting a part or all of the hydrogen atoms in these groups with a halogen atom. Of them, dimethylamino, diethylamino, pyrrolidinyl and piperidinyl groups, and groups obtained by substituting a part or all of the hydrogen atoms in these groups with a halogen atom are preferable.

Of R¹, R², R³ and R⁴, two groups bonded to two adjoining carbon atoms may be bonded to each other to form a ring together with the carbon atoms to which the two groups are bonded, of R⁵, R⁶, R⁷, R⁸ and R⁹, two groups bonded to two adjoining carbon atoms may be bonded to each other to form a ring together with the carbon atoms to which the two groups are bonded, of R¹², R¹³, R¹⁴, R¹⁵, and R¹⁶, two group bonded to two adjoining carbon atoms may be bonded to each other to form a ring together with the two carbon atoms to which the two groups are bonded, of R¹⁷, R¹⁸, R¹⁹, R²⁰ and R²¹, two group bonded to two adjoining carbon atoms may be bonded to each other to form a ring together with the two carbon atoms to which the two groups are bonded, and R¹⁰ and R¹¹ may be bonded to each other to form a ring together with the silicon atom to which they are bonded. In this context, the ring is a saturated or unsaturated hydrocarbyl ring substituted by a hydrocarbyl group having 1 to 20 carbon atoms, a saturated or unsaturated silahydrocarbyl ring substituted by a hydrocarbyl group having 1 to 20 carbon atoms, etc. Examples thereof include cyclopropane, cyclopropane, cyclobutane, cyclobutene, cyclopentane, cyclopentene, cyclohexane, cyclohexene, cycloheptane, cycloheptene, cyclooctane, cyclooctene, benzene, naphthalene, anthracene, silacyclopropane, silacyclobutane, silacyclopentane and silacyclohexane rings.

In the transition metal complexes (1-1) to (1-3), R¹, R², R³ and R⁴ are each independently preferably a hydrogen atom, a halogen atom, an alkyl group having 1 to 20 carbon atoms, an aryl group having 6 to 20 carbon atoms or an aralkyl group having 7 to 20 carbon atoms, more preferably an alkyl group having 1 to 20 carbon atoms, even more preferably a methyl group.

Examples of R¹, R², R³ and R⁴ include the following substructures represented by substructural formula (2):

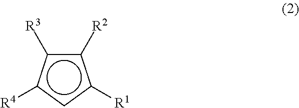

wherein R¹, R², R³ and R⁴ are as defined above:

cyclopentadienyl, methylcyclopentadienyl, ethylcyclopentadienyl, n-propylcyclopentadienyl, isopropylcyclopentadienyl, n-butylcyclopentadienyl, sec-butylcyclopentadienyl, tert-butylcyclopentadienyl, dimethylcyclopentadienyl, trimethylcyclopentadienyl, tetramethylcyclopentadienyl, phenylcyclopentadienyl, benzylcyclopentadienyl, indenyl, fluorenyl, tetrahydroindenyl, methyltetrahydroindenyl, dimethyltetrahydroindenyl and octahydrofluorenyl.

Of the cyclopentadienyl substructures exemplified above, a preferable cyclopentadienyl substructure is tetramethylcyclopentadienyl, etc.

In the transition metal complexes (1-1) to (1-3), R⁵, R⁶, R⁷, R⁸, R⁹, R¹², R¹³, R¹⁴, R¹⁵, R¹⁶, R¹⁷, R¹⁸, R¹⁹, R²⁰ and R²¹ are each independently preferably a hydrogen atom, a halogen atom, an alkyl group having 1 to 20 carbon atoms, an aryl group having 6 to 20 carbon atoms, or an aralkyl group having 7 to 20 carbon atoms.

In the transition metal complexes (1-1) to (1-3), R⁶, R⁸, R¹³, R¹⁵, R¹⁸ and R²⁰ are preferably an alkyl group having 1 to 20 carbon atoms which may have a halogen atom as a substituent.

Examples of a preferable combination of the groups represented by R⁵, R⁶, R⁷, R⁸ and R⁹ include the following substructures represented by substructural formula (3):

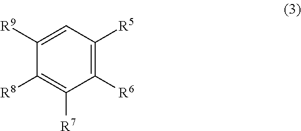

wherein. R⁵, R⁶, R⁷, R⁸ and R⁹ are as defined above:

Examples of a preferable combination of the groups represented by R¹², R¹³, R¹⁴, R¹⁵ and R¹⁶ each independently include the following substructures represented by substructural formula (4):

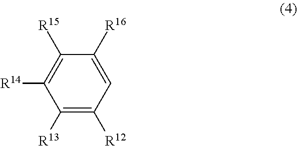

wherein R¹², R¹³, R¹⁴, R¹⁵ and R¹⁶ are as defined above:

and examples of a preferable combination of the groups represented by R¹⁷, R¹⁸, R¹⁹, R²⁰ and R²¹ include the following substructures represented by substructural formula (5):

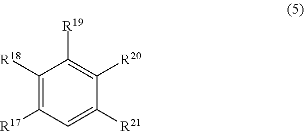

wherein R¹⁷, R¹⁸, R¹⁹, R²⁰ and R²¹ are as defined above:

phenyl, methylphenyl, dimethylphenyl, trimethylphenyl, tetramethylphenyl, pentamethylphenyl, ethylphenyl, diethylphenyl, isopropylphenyl, diisopropylphenyl, tert-butylphenyl, di-tert-butylphenyl, tert-butylmethylphenyl, di(tert-butyl)methylphenyl, naphthyl, anthracenyl, chlorophenyl, dichlorophenyl, fluorophenyl, pentafluorophenyl, bis(trifluoromethyl)phenyl, biphenyl, benzylphenyl, methoxyphenyl, phenoxyphenyl, benzyloxyphenyl, trimethylsilylphenyl and dimethylaminophenyl.

Of the substructures exemplified above, a more preferable substructure is phenyl, methylphenyl, dimethylphenyl, trimethylphenyl, diethylphenyl, trimethylsilylphenyl, etc.

In the transition metal complexes (1-1) and (1-2), R¹⁰ and R¹¹ are each independently preferably a hydrogen atom, an alkyl group having 1 to 20 carbon atoms or an aralkyl group having 7 to 20 carbon atoms, and examples thereof include methyl, ethyl, n-propyl, isopropyl, n-butyl, sec-butyl, tert-butyl or benzyl group.

Examples of a preferable combination of the groups represented by R¹⁰, R¹¹ and J include the following substructures represented by substructural formula (6):

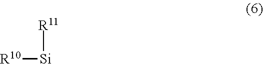

wherein R¹⁰ and R¹¹ are as defined above:

dimethylsilylene, diethylsilylene, ethylmethylsilylene, di(n-propyl)silylene, methyl(n-propyl)silylene, di(n-butyl)silylene, n-butylmethylsilylene, n-hexylmethylsilylene, methyl(n-octyl)silylene, n-decylmethylsilylene, methyl(n-octadecyl)silylene, cyclohexylmethylsilylene and cyclotetramethylenesilylene.

Preferable examples thereof include a structural formula represented by substructural formula (6), wherein

R¹⁰ and R¹¹ are the same as each other and are an alkyl group having 1 to 20 carbon atoms which may have a halogen atom as a substituent.

Specifically, the substructure is dimethylsilylene, diethylsilylene, ethylmethylsilylene, n-butylmethylsilylene, cyclohexylmethylsilylene, cyclotetramethylenesilylene, etc.

Examples of a preferable combination of the groups represented by R¹⁰, R¹¹ and J include the following substructures represented by substructural formula (7):

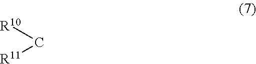

wherein R¹⁰ and R¹¹ are as defined above:

isopropylidene, 1-ethylpropylene, 1-methylpropylene, 1-n-propylbutylene, 1-methylbutylene, 1-n-butylpentylene, 1-methylpentylene, 1-methylheptylene, 1-methylnonylene, 1-methyldodecylene, 1-methylnonadecylene, 1-cyclohexylethylene, cyclotetramethylenemethylene, diphenylmethylene, 1-phenylethylene, di(3,5-dimethylphenyl)methylene, di(3,5-diethylphenyl)methylene, (3,5-dimethylphenyl)(3,5-di-tert-butylphenyl)methylene, di(3,5-diphenylphenyl)methylene and di(3,5-dibenzylphenyl)methylene.

Preferable examples thereof include a structural formula represented by substructural formula (7), wherein

R¹⁰ and R¹¹ are the same as each other and are an alkyl group having 1 to 20 carbon atoms which may have a halogen atom as a substituent.

Specifically, the substructure is isopropylidene, 1-ethylpropylene, 1-methylpropylene, 1-methylpentylene, 1-cyclohexylethylene, etc.

In the transition metal complexes (1-1) to (1-3), X¹, X² and X³ are each independently preferably a halogen atom, an alkyl group having 1 to 20 carbon atoms which may have a halogen atom as a substituent or an aralkyl group having 7 to 20 carbon atoms which may have a halogen atom as a substituent, more preferably a halogen atom, or an alkyl group having 1 to 20 carbon atoms which may have a halogen atom as a substituent.

Examples of the transition metal complex (1-1) wherein J is a silicon atom include the following complexes:

[1-dimethylphenylsilyl-2,3,4,5-tetramethylcyclopentadienyl]titanium trichloride, [1-diethylphenylsilyl-2,3,4,5-tetramethylcyclopentadienyl]titanium trichloride, [1-phenyldi(n-propyl)silyl-2,3,4,5-tetramethylcyclopentadienyl]titanium trichloride, [1-diisopropylphenylsilyl-2,3,4,5-tetramethylcyclopentadienyl]titanium trichloride, [1-di(n-butyl)phenylsilyl-2,3,4,5-tetramethylcyclopentadienyl]titanium trichloride, [1-di(isobutyl)phenylsilyl-2,3,4,5-tetramethylcyclopentadienyl]titanium trichloride, [1-di(sec-butyl)phenylsilyl-2,3,4,5-tetramethylcyclopentadienyl]titanium trichloride, [1-di(tert-butyl)phenylsilyl-2,3,4,5-tetramethylcyclopentadienyl]titanium trichloride, [1-ethylmethylphenylsilyl-2,3,4,5-tetramethylcyclopentadienyl]titanium trichloride, [1-methylphenyl(n-propyl)silyl-2,3,4,5-tetramethylcyclopentadienyl]titanium trichloride, [1-methylphenyl)isopropyl)silyl-2,3,4,5-tetramethylcyclopentadienyl]titanium trichloride, [1-n-butylmethylphenylsilyl-2,3,4,5-tetramethylcyclopentadienyl]titanium trichloride, [1-isobutylmethylphenylsilyl-2,3,4,5-tetramethylcyclopentadienyl]titanium trichloride, [1-sec-butylmethylphenylsilyl-2,3,4,5-tetramethylcyclopentadienyl]titanium trichloride, [1-tert-butylmethylphenylsilyl-2,3,4,5-tetramethylcyclopentadienyl]titanium trichloride, [1-cyclohexylmethylphenylsilyl-2,3,4,5-tetramethylcyclopentadienyl]titanium trichloride, [1-methyl(n-octadecyl)phenylsilyl-2,3,4,5-tetramethylcyclopentadienyl]titanium trichloride,

[1-dimethyl(3,5-dimethylphenyl)silyl-2,3,4,5-tetramethylcyclopentadienyl]titanium trichloride, [1-diethyl(3,5-dimethylphenyl)silyl-2,3,4,5-tetramethylcyclopentadienyl]titanium trichloride, [1-(3,5-dimethylphenyl)di(n-propyl)silyl-2,3,4,5-tetramethylcyclopentadienyl]titanium trichloride, [1-diisopropyl(3,5-dimethylphenyl)silyl-2,3,4,5-tetramethylcyclopentadienyl]titanium trichloride, [1-di(n-butyl)(3,5-dimethylphenyl)silyl-2,3,4,5-tetramethylcyclopentadienyl]titanium trichloride, [1-di(isobutyl)(3,5-dimethylphenyl)silyl-2,3,4,5-tetramethylcyclopentadienyl]titanium trichloride, [1-di(sec-butyl)(3,5-dimethylphenyl)silyl-2,3,4,5-tetramethylcyclopentadienyl]titanium trichloride, [1-di(tert-butyl)(3,5-dimethylphenyl)silyl-2,3,4,5-tetramethylcyclopentadienyl]titanium trichloride, [1-ethylmethyl(3,5-dimethylphenyl)silyl-2,3,4,5-tetramethylcyclopentadienyl]titanium trichloride, [1-methyl(3,5-dimethylphenyl)(n-propyl)silyl-2,3,4,5-tetramethylcyclopentadienyl]titanium trichloride, [1-methyl(3,5-dimethylphenyl)(isopropyl)silyl-2,3,4,5-tetramethylcyclopentadienyl]titanium trichloride, [1-n-butylmethyl(3,5-dimethylphenyl)silyl-2,3,4,5-tetramethylcyclopentadienyl]titanium trichloride, [1-isobutylmethyl(3,5-dimethylphenyl)silyl-2,3,4,5-tetramethylcyclopentadienyl]titanium trichloride, [1-sec-butylmethyl(3,5-dimethylphenyl)silyl-2,3,4,5-tetramethylcyclopentadienyl]titanium trichloride, [1-tert-butylmethyl(3,5-dimethylphenyl)silyl-2,3,4,5-tetramethylcyclopentadienyl]titanium trichloride, [1-cyclohexylmethyl(3,5-dimethylphenyl)silyl-2,3,4,5-tetramethylcyclopentadienyl]titanium trichloride and [1-methyl(n-octadecyl)(3,5-dimethylphenyl)silyl-2,3,4,5-tetramethylcyclopentadienyl]titanium trichloride.

Moreover, examples of the transition metal complex (1-1) wherein J is a silicon atom also include titanium chloride complexes obtained by substituting “cyclopentadienyl”, “2-methylcyclopentadienyl”, “3-methylcyclopentadienyl”, “2,3-dimethylcyclopentadienyl”, “2,4-dimethylcyclopentadienyl”, “2,5-dimethylcyclopentadienyl”, “2,3,5-trimethylcyclopentadienyl”, “2-ethylcyclopentadienyl”, “3-ethylcyclopentadienyl”, “2-n-propylcyclopentadienyl”, “3-n-propylcyclopentadienyl”, “2-isopropylcyclopentadienyl”, “3-isopropylcyclopentadienyl”, “2-n-butylcyclopentadienyl”, “3-n-butylcyclopentadienyl”, “2-sec-butylcyclopentadienyl”, “3-sec-butylcyclopentadienyl”, “2-tert-butylcyclopentadienyl”, “3-tert-butylcyclopentadienyl”, “2-phenylcyclopentadienyl”, “3-phenylcyclopentadienyl”, “2-benzylcyclopentadienyl”, “3-benzylcyclopentadienyl”, “indenyl”, “2-methylindenyl”, “fluorenyl”, “tetrahydroindenyl”, “2-methyltetrahydroindenyl” and “octahydrofluorenyl” for “2,3,4,5-tetramethylcyclopentadienyl” in the titanium chloride complexes exemplified above.

Furthermore, examples of the transition metal complex (1-1) wherein J is a silicon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Examples of the transition metal complex (1-1) wherein J is a carbon atom include the following complexes:

(1-phenylmethyl-cyclopentadienyl)titanium trichloride, [142-methylphenyl)methyl-cyclopentadienyl]titanium trichloride, [1-(3-methylphenyl)methyl-cyclopentadienyl]titanium trichloride, [1-(4-methylphenyl)methyl-cyclopentadienyl]titanium trichloride, [1-(2,3-dimethylphenyl)methyl-cyclopentadienyl]titanium trichloride, [1-(2,4-dimethylphenyl)methyl-cyclopentadienyl]titanium trichloride, [1-(2,5-dimethylphenyl)methyl-cyclopentadienyl]titanium trichloride, [1-(2,6-dimethylphenyl)methyl-cyclopentadienyl]titanium trichloride, [1-(3,4-dimethylphenyl)methyl-cyclopentadienyl]titanium trichloride, [1-(3,5-dimethylphenyl)methyl-cyclopentadienyl]titanium trichloride, [1-(2,3,4-trimethylphenyl)methyl-cyclopentadienyl]titanium trichloride, [1-(2,3,5-trimethylphenyl)methyl-cyclopentadienyl]titanium trichloride, [1-(2,3,6-trimethylphenyl)methyl-cyclopentadienyl]titanium trichloride, [1-(2,4,5-trimethylphenyl)methyl-cyclopentadienyl]titanium trichloride, [1-(2,4,6-trimethylphenyl)methyl-cyclopentadienyl]titanium trichloride, [1-(3,4,5-trimethylphenyl)methyl-cyclopentadienyl]titanium trichloride, [1-(2,3,4,5,6-pentamethylphenyl)methyl-cyclopentadienyl]titanium trichloride, [1-(2-trimethylsilylphenyl)methyl-cyclopentadienyl]titanium trichloride, [1-(3-trimethylsilylphenyl)methyl-cyclopentadienyl]titanium trichloride, [1-(4-trimethylsilylphenyl)methyl-cyclopentadienyl]titanium trichloride, [1-(1-methyl-1-phenylethyl)-cyclopentadienyl]titanium trichloride, [1-(1-methyl-1-(2-methylphenyl)ethyl)-cyclopentadienyl]titanium trichloride, [1-(1-methyl-1-(3-methylphenyl)ethyl)-cyclopentadienyl]titanium trichloride, [1-(1-methyl-1-(4-methylphenyl)ethyl)-cyclopentadienyl]titanium trichloride, [1-(1-methyl-1-(2,3-dimethylphenyl)ethyl)-cyclopentadienyl]titanium trichloride, [1-(1-methyl-1-(2,4-dimethylphenyl)ethyl)-cyclopentadienyl]titanium trichloride, [1-(1-methyl-1-(2,5-dimethylphenyl)ethyl)-cyclopentadienyl]titanium trichloride, [1-(1-methyl-1-(2,6-dimethylphenyl)ethyl)-cyclopentadienyl]titanium trichloride, [1-(1-methyl-1-(3,4-dimethylphenyl)ethyl)-cyclopentadienyl]titanium trichloride, [1-(1-methyl-1-(3,5-dimethylphenyl)ethyl)-cyclopentadienyl]titanium trichloride, [1-(1-methyl-1-(2,3,4-trimethylphenyl)ethyl)-cyclopentadienyl]titanium trichloride, [1-(1-methyl-1-(2,3,5-trimethylphenyl)ethyl)-cyclopentadienyl]titanium trichloride, [1-(1-methyl-1-(2,3,6-trimethylphenyl)ethyl)-cyclopentadienyl]titanium trichloride, [1-(1-methyl-1-(2,4,5-trimethylphenyl)ethyl)-cyclopentadienyl]titanium trichloride, [1-(1-methyl-1-(2,4,6-trimethylphenyl)ethyl)-cyclopentadienyl]titanium trichloride, [1-(1-methyl-1-(3,4,5-trimethylphenyl)ethyl)-cyclopentadienyl]titanium trichloride, [1-(1-methyl-1-(2,3,4,5,6-pentamethylphenyl)ethyl)-cyclopentadienyl]titanium trichloride, [1-(1-ethyl-1-phenylpropyl)-cyclopentadienyl]titanium trichloride, [1-(1-phenylcyclohexyl)-cyclopentadienyl]titanium trichloride, [1-(1-phenylvinyl)-cyclopentadienyl]titanium trichloride,

[1-phenylmethyl-3-trimethylsilylcyclopentadienyl]titanium trichloride, [1-(2-methylphenyl)methyl-3-trimethylsilylcyclopentadienyl]titanium trichloride, [1-(3-methylphenyl)methyl-3-trimethylsilylcyclopentadienyl]titanium trichloride, [1-(4-methylphenyl)methyl-3-trimethylsilylcyclopentadienyl]titanium trichloride, [1-(2,3-dimethylphenyl)methyl-3-trimethylsilylcyclopentadienyl]titanium trichloride, [1-(2,4-dimethylphenyl)methyl-3-trimethylsilylcyclopentadienyl]titanium trichloride, [1-(2,5-dimethylphenyl)methyl-3-trimethylsilylcyclopentadienyl]titanium trichloride, [1-(2,6-dimethylphenyl)methyl-3-trimethylsilylcyclopentadienyl]titanium trichloride, [1-(3,4-dimethylphenyl)methyl-3-trimethylsilylcyclopentadienyl]titanium trichloride, [1-(3,5-dimethylphenyl)methyl-3-trimethylsilylcyclopentadienyl]titanium trichloride, [1-(2,3,4-trimethylphenyl)methyl-3-trimethylsilylcyclopentadienyl]titanium trichloride, [1-(2,3,5-trimethylphenyl)methyl-3-trimethylsilylcyclopentadienyl]titanium trichloride, [1-(2,3,6-trimethylphenyl)methyl-3-trimethylsilylcyclopentadienyl]titanium trichloride, [1-(2,4,5-trimethylphenyl)methyl-3-trimethylsilylcyclopentadienyl]titanium trichloride, [1-(2,4,6-trimethylphenyl)methyl-3-trimethylsilylcyclopentadienyl]titanium trichloride, [1-(3,4,5-trimethylphenyl)methyl-3-trimethylsilylcyclopentadienyl]titanium trichloride, [1-(2,3,4,5,6-pentamethylphenyl)methyl-3-trimethylsilylcyclopentadienyl]titanium trichloride, [1-(2-trimethylsilylphenyl)methyl-3-trimethylsilylcyclopentadienyl]titanium trichloride, [1-(3-trimethylsilylphenyl)methyl-3-trimethylsilylcyclopentadienyl]titanium trichloride, [1-(4-trimethylsilylphenyl)methyl-3-trimethylsilylcyclopentadienyl]titanium trichloride, [1-(1-methyl-1-phenylethyl)-3-trimethylsilylcyclopentadienyl]titanium trichloride, [1-(1-methyl-1-(2-methylphenyl)ethyl)-3-trimethylsilylcyclopentadienyl]titanium trichloride, [1-(1-methyl-1-(3-methylphenyl)ethyl)-3-trimethylsilylcyclopentadienyl]titanium trichloride, [1-(1-methyl-1-(4-methylphenyl)ethyl)-3-trimethylsilylcyclopentadienyl]titanium trichloride, [1-(1-methyl-1-(2,3-dimethylphenyl)ethyl)-3-trimethylsilylcyclopentadienyl]titanium trichloride, [1-(1-methyl-1-(2,4-dimethylphenyl)ethyl)-3-trimethylsilylcyclopentadienyl]titanium trichloride, [1-(1-methyl-1-(2,5-dimethylphenyl)ethyl)-3-trimethylsilylcyclopentadienyl]titanium trichloride, [1-(1-methyl-1-(2,6-dimethylphenyl)ethyl)-3-trimethylsilylcyclopentadienyl]titanium trichloride, [1-(1-methyl-1-(3,4-dimethylphenyl)ethyl)-3-trimethylsilylcyclopentadienyl]titanium trichloride,

[1-(1-methyl-1-(3,5-dimethylphenyl)ethyl)-3-trimethylsilylcyclopentadienyl]titanium trichloride, [1-(1-methyl-1-(2,3,4-trimethylphenyl)ethyl)-3-trimethylsilylcyclopentadienyl]titanium trichloride, [1-(1-methyl-1-(2,3,5-trimethylphenyl)ethyl)-3-trimethylsilylcyclopentadienyl]titanium trichloride, [1-(1-methyl-1-(2,3,6-trimethylphenyl)ethyl)-3-trimethylsilylcyclopentadienyl]titanium trichloride, [1-(1-methyl-1-(2,4,5-trimethylphenyl)ethyl)-3-trimethylsilylcyclopentadienyl]titanium trichloride, [1-(1-methyl-1-(2,4,6-trimethylphenyl)ethyl)-3-trimethylsilylcyclopentadienyl]titanium trichloride, [1-(1-methyl-1-(3,4,5-trimethylphenyl)ethyl)-3-trimethylsilylcyclopentadienyl]titanium trichloride, [1-(1-methyl-1-(2,3,4,5,6-pentamethylphenyl)ethyl)-3-trimethylsilylcyclopentadienyl]titanium trichloride, [1-(1-ethyl-1-phenylpropyl)-3-trimethylsilylcyclopentadienyl]titanium trichloride, [1-(1-phenylcyclohexyl)-3-trimethylsilyl cyclopentadienyl]titanium trichloride and [1-(1-phenylvinyl)-3-trimethylsilylcyclopentadienyl]titanium trichloride.

Moreover, examples of the transition metal complex (1-1) wherein J is a carbon atom also include titanium chloride complexes obtained by substituting 2-methylcyclopentadienyl, 3-methylcyclopentadienyl, 2,3-dimethylcyclopentadienyl, 2,4-dimethylcyclopentadienyl, 2,5-dimethylcyclopentadienyl, 2,3,4-trimethylcyclopentadienyl, 2,3,5-trimethylcyclopentadienyl, 2,3,4,5-tetramethylcyclopentadienyl, 2-n-butylcyclopentadienyl, 3-n-butylcyclopentadienyl, 2,3-di-n-butylcyclopentadienyl, 2,4-di-n-butylcyclopentadienyl, 2,5-di-n-butylcyclopentadienyl, 2,3,4-tri-n-butylcyclopentadienyl, 2,3,5-tri-n-butylcyclopentadienyl, 2,3,4,5-tetra-n-butylcyclopentadienyl, 2-tert-butylcyclopentadienyl, 3-tert-butylcyclopentadienyl, 2,3-di-tert-butylcyclopentadienyl, 2,4-di-tert-butylcyclopentadienyl, 2,5-di-tert-butylcyclopentadienyl, 2,3,4-tri-tert-butylcyclopentadienyl, 2,3,5-tri-tert-butylcyclopentadienyl, 2,3,4,5-tetra-tert-butylcyclopentadienyl, 2-trimethylsilylcyclopentadienyl, 3-trimethylsilylcyclopentadienyl, 2,3-di-trimethylsilylcyclopentadienyl, 2,4-di-trimethylsilylcyclopentadienyl, 2,5-di-trimethylsilylcyclopentadienyl, 2,3,4-tris(trimethylsilyl)cyclopentadienyl, 2,3,5-tris(trimethylsilyl)cyclopentadienyl, 2,3,4,5-tetrakis(trimethylsilyl)cyclopentadienyl, indenyl or fluorenyl for cyclopentadienyl in the titanium chloride complexes exemplified above.

Furthermore, examples of the transition metal complex (1-1) wherein J is a carbon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Examples of the transition metal complex (1-2) wherein J is a silicon atom include the following complexes:

[1-methyldiphenylsilyl-2,3,4,5-tetramethylcyclopentadienyl]titanium trichloride, [1-ethyldiphenylsilyl-2,3,4,5-tetramethylcyclopentadienyl]titanium trichloride, [1-n-propyl diphenylsilyl-2,3,4,5-tetramethylcyclopentadienyl]titanium trichloride, [1-isopropyl diphenylsilyl-2,3,4,5-tetramethylcyclopentadienyl]titanium trichloride, [1-n-butyl diphenylsilyl-2,3,4,5-tetramethylcyclopentadienyl]titanium trichloride, [1-isobutyl diphenylsilyl-2,3,4,5-tetramethylcyclopentadienyl]titanium trichloride, [1-sec-butyl diphenylsilyl-2,3,4,5-tetramethylcyclopentadienyl]titanium trichloride, [1-tert-butyl diphenylsilyl-2,3,4,5-tetramethylcyclopentadienyl]titanium trichloride, [1-cyclohexyl diphenylsilyl-2,3,4,5-tetramethylcyclopentadienyl]titanium trichloride, [1-n-octadecyl diphenylsilyl-2,3,4,5-tetramethylcyclopentadienyl]titanium trichloride, [1-methylphenyl(2-methylphenyl)silyl-2,3,4,5-tetramethylcyclopentadienyl]titanium trichloride, [1-methylphenyl(3-methylphenyl)silyl-2,3,4,5-tetramethylcyclopentadienyl]titanium trichloride, [1-methylphenyl(4-methylphenyl)silyl-2,3,4,5-tetramethylcyclopentadienyl]titanium trichloride, [1-methylphenyl(2,3-dimethylphenyl)silyl-2,3,4,5-tetramethylcyclopentadienyl]titanium trichloride, [1-methylphenyl(2,4-dimethylphenyl)silyl-2,3,4,5-tetramethylcyclopentadienyl]titanium trichloride, [1-methylphenyl(2,5-dimethylphenyl)silyl-2,3,4,5-tetramethylcyclopentadienyl]titanium trichloride, [1-methylphenyl(2,6-dimethylphenyl)silyl-2,3,4,5-tetramethylcyclopentadienyl]titanium trichloride, [1-methylphenyl(3,5-dimethylphenyl)silyl-2,3,4,5-tetramethylcyclopentadienyl]titanium trichloride, [1-methylphenyl(3,4,5-trimethylphenyl)silyl-2,3,4,5-tetramethylcyclopentadienyl]titanium trichloride,

[1-ethylphenyl(3,5-dimethylphenyl)silyl-2,3,4,5-tetramethylcyclopentadienyl]titanium trichloride, [1-n-propylphenyl(3,5-dimethylphenyl)silyl-2,3,4,5-tetramethylcyclopentadienyl]titanium trichloride, [1-isopropylphenyl(3,5-dimethylphenyl)silyl-2,3,4,5-tetramethylcyclopentadienyl]titanium trichloride, [1-n-butylphenyl(3,5-dimethylphenyl)silyl-2,3,4,5-tetramethylcyclopentadienyl]titanium trichloride, [1-isobutylphenyl(3,5-dimethylphenyl)silyl-2,3,4,5-tetramethylcyclopentadienyl]titanium trichloride, [1-sec-butylphenyl(3,5-dimethylphenyl)silyl-2,3,4,5-tetramethylcyclopentadienyl]titanium trichloride, [1-tert-butylphenyl(3,5-dimethylphenyl)silyl-2,3,4,5-tetramethylcyclopentadienyl]titanium trichloride, [1-cyclohexylphenyl(3,5-dimethylphenyl)silyl-2,3,4,5-tetramethylcyclopentadienyl]titanium trichloride, [1-n-octadecylphenyl(3,5-dimethylphenyl)silyl-2,3,4,5-tetramethylcyclopentadienyl]titanium trichloride, [1-methyl(2-methylphenyl)(3,5-dimethylphenyl)silyl-2,3,4,5-tetramethylcyclopentadienyl]titanium trichloride, [1-methyl(3-methylphenyl)(3,5-dimethylphenyl)silyl-2,3,4,5-tetramethylcyclopentadienyl]titanium trichloride, [1-methyl(4-methylphenyl)(3,5-dimethylphenyl)silyl-2,3,4,5-tetramethylcyclopentadienyl]titanium trichloride, [1-methyl(2,3-dimethylphenyl)(3,5-dimethylphenyl)silyl-2,3,4,5-tetramethylcyclopentadienyl]titanium trichloride, [1-methyl(2,4-dimethylphenyl)(3,5-dimethylphenyl)silyl-2,3,4,5-tetramethylcyclopentadienyl]titanium trichloride, [1-methyl(2,5-dimethylphenyl)(3,5-dimethylphenyl)silyl-2,3,4,5-tetramethylcyclopentadienyl]titanium trichloride, [1-methylphenyl(2,6-dimethylphenyl)(3,5-dimethylphenyl)silyl-2,3,4,5-tetramethylcyclopentadienyl]titanium trichloride, [1-methylbis(3,5-dimethylphenyl)silyl-2,3,4,5-tetramethylcyclopentadienyl]titanium trichloride and [1-methyl(3,5-dimethylphenyl)(3,4,5-trimethylphenyl)silyl-2,3,4,5-tetramethylcyclopentadienyl]titanium trichloride.

Moreover, examples of the transition metal complex (1-2) wherein J is a silicon atom also include titanium chloride complexes obtained by substituting “cyclopentadienyl”, “2-methylcyclopentadienyl”, “3-methylcyclopentadienyl”, “2,3-dimethylcyclopentadienyl”, “2,4-dimethylcyclopentadienyl”, “2,5-dimethylcyclopentadienyl”, “2,3,5-trimethylcyclopentadienyl”, “2-ethylcyclopentadienyl”, “3-ethylcyclopentadienyl”, “2-n-propylcyclopentadienyl”, “3-n-propylcyclopentadienyl”, “2-isopropylcyclopentadienyl”, “3-isopropylcyclopentadienyl”, “2-n-butylcyclopentadienyl”, “3-n-butylcyclopentadienyl”, “2-sec-butylcyclopentadienyl”, “3-sec-butylcyclopentadienyl”, “2-tert-butylcyclopentadienyl”, “3-tert-butylcyclopentadienyl”, “2-phenylcyclopentadienyl”, “3-phenylcyclopentadienyl”, “2-benzylcyclopentadienyl”, “3-benzylcyclopentadienyl”, “indenyl”, “2-methylindenyl”, “fluorenyl”, “tetrahydroindenyl”, “2-methyltetrahydroindenyl” or “octahydrofluorenyl” for “2,3,4,5-tetramethylcyclopentadienyl” in the titanium chloride complexes exemplified above.

Furthermore, examples of the transition metal complex (1-2) wherein J is a silicon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Examples of the transition metal complex (1-2) wherein J is a carbon atom include the following complexes:

[1-diphenylmethyl-cyclopentadienyl]titanium trichloride, [1-diphenylmethyl-2-methyl-cyclopentadienyl]titanium trichloride, [1-diphenylmethyl-3-methyl-cyclopentadienyl]titanium trichloride, [1-diphenylmethyl-2,3-dimethyl-cyclopentadienyl]titanium trichloride, [1-diphenylmethyl-2,4-dimethyl-cyclopentadienyl]titanium trichloride, [1-diphenylmethyl-3,4-dimethyl-cyclopentadienyl]titanium trichloride, [1-diphenylmethyl-2,3,4-trimethyl-cyclopentadienyl]titanium trichloride, [1-diphenylmethyl-2,3,5-trimethyl-cyclopentadienyl]titanium trichloride, [1-diphenylmethyl-2,3,4,5-tetramethyl-cyclopentadienyl]titanium trichloride, [1-diphenylmethyl-2-ethyl-cyclopentadienyl]titanium trichloride, [1-diphenylmethyl-3-ethyl-cyclopentadienyl]titanium trichloride, [1-diphenylmethyl-2-n-propyl-cyclopentadienyl]titanium trichloride, [1-diphenylmethyl-3-n-propyl-cyclopentadienyl]titanium trichloride, [1-diphenylmethyl-2-isopropyl-cyclopentadienyl]titanium trichloride, [1-diphenylmethyl-3-isopropyl-cyclopentadienyl]titanium trichloride, [1-diphenylmethyl-2-n-butyl-cyclopentadienyl]titanium trichloride, [1-diphenylmethyl-3-n-butyl-cyclopentadienyl]titanium trichloride, [1-diphenylmethyl-2-sec-butyl-cyclopentadienyl]titanium trichloride, [1-diphenylmethyl-3-sec-butyl-cyclopentadienyl]titanium trichloride, [1-diphenylmethyl-2-tert-butyl-cyclopentadienyl]titanium trichloride, [1-diphenylmethyl-3-tert-butyl-cyclopentadienyl]titanium trichloride, [1-diphenylmethyl-2-phenyl-cyclopentadienyl]titanium trichloride, [1-diphenylmethyl-3-phenyl-cyclopentadienyl]titanium trichloride, [1-diphenylmethyl-2-benzyl-cyclopentadienyl]titanium trichloride, [1-diphenylmethyl-3-benzyl-cyclopentadienyl]titanium trichloride, [1-diphenylmethyl-indenyl]titanium trichloride, [1-diphenylmethyl-2-methylindenyl]titanium trichloride, [1-diphenylmethyl-tetrahydroindenyl]titanium trichloride, [9-diphenylmethyl-fluorenyl]titanium trichloride, [1-diphenylmethyl-2-methyltetrahydroindenyl]titanium trichloride, [9-diphenylmethyl-octahydrofluorenyl]titanium trichloride, [3-diphenylmethyl-1-trimethylsilyl-cyclopentadienyl]titanium trichloride,

[1-di(3-methylphenyl)methyl-cyclopentadienyl]titanium trichloride, [1-di(3-methylphenyl)methyl-2-methyl-cyclopentadienyl]titanium trichloride, [1-di(3-methylphenyl)methyl-3-methyl-cyclopentadienyl]titanium trichloride, [1-di(3-methylphenyl)methyl-2,3-dimethyl-cyclopentadienyl]titanium trichloride, [1-di(3-methylphenyl)methyl-2,4-dimethyl-cyclopentadienyl]titanium trichloride, [1-di(3-methylphenyl)methyl-3,4-dimethyl-cyclopentadienyl]titanium trichloride, [1-di(3-methylphenyl)methyl-2,3,4-trimethyl-cyclopentadienyl]titanium trichloride, [1-di(3-methylphenyl)methyl-2,3,5-trimethyl-cyclopentadienyl]titanium trichloride, [1-di(3-methylphenyl)methyl-2,3,4,5-tetramethyl-cyclopentadienyl]titanium trichloride, [1-di(3-methylphenyl)methyl-2-ethyl-cyclopentadienyl]titanium trichloride, [1-di(3-methylphenyl)methyl-3-ethyl-cyclopentadienyl]titanium trichloride, [1-di(3-methylphenyl)methyl-2-n-propyl-cyclopentadienyl]titanium trichloride, [1-di(3-methylphenyl)methyl-3-n-propyl-cyclopentadienyl]titanium trichloride, [1-di(3-methylphenyl)methyl-2-isopropyl-cyclopentadienyl]titanium trichloride, [1-di(3-methylphenyl)methyl-3-isopropyl-cyclopentadienyl]titanium trichloride, [1-di(3-methylphenyl)methyl-2-n-butyl-cyclopentadienyl]titanium trichloride, [1-di(3-methylphenyl)methyl-3-n-butyl-cyclopentadienyl]titanium trichloride, [1-di(3-methylphenyl)methyl-2-sec-butyl-cyclopentadienyl]titanium trichloride, [1-di(3-methylphenyl)methyl-3-sec-butyl-cyclopentadienyl]titanium trichloride, [1-di(3-methylphenyl)methyl-2-tert-butyl-cyclopentadienyl]titanium trichloride, [1-di(3-methylphenyl)methyl-3-tert-butyl-cyclopentadienyl]titanium trichloride, [1-di(3-methylphenyl)methyl-2-phenyl-cyclopentadienyl]titanium trichloride, [1-di(3-methylphenyl)methyl-3-phenyl-cyclopentadienyl]titanium trichloride, [1-di(3-methylphenyl)methyl-2-benzyl-cyclopentadienyl]titanium trichloride, [1-di(3-methylphenyl)methyl-3-benzyl-cyclopentadienyl]titanium trichloride, [1-di(3-methylphenyl)methyl-indenyl]titanium trichloride, [1-di(3-methylphenyl)methyl-2-methylindenyl]titanium trichloride, [9-di(3-methylphenyl)methyl-fluorenyl]titanium trichloride, [1-di(3-methylphenyl)methyl-tetrahydroindenyl]titanium trichloride, [1-di(3-methylphenyl)methyl-2-methyltetrahydroindenyl]titanium trichloride, [9-di(3-methylphenyl)methyl-octahydrofluorenyl]titanium trichloride, [3-di(3-methylphenyl)methyl-1-trimethylsilyl-cyclopentadienyl]titanium trichloride,

[1-bis(3,5-dimethylphenyl)methyl-cyclopentadienyl]titanium trichloride, [1-bis(3,5-dimethylphenyl)methyl-2-methyl-cyclopentadienyl]titanium trichloride, [1-bis(3,5-dimethylphenyl)methyl-3-methyl-cyclopentadienyl]titanium trichloride, [1-bis(3,5-dimethylphenyl)methyl-2,3-dimethyl-cyclopentadienyl]titanium trichloride, [1-bis(3,5-dimethylphenyl)methyl-2,4-dimethyl-cyclopentadienyl]titanium trichloride, [1-bis(3,5-dimethylphenyl)methyl-3,4-dimethyl-cyclopentadienyl]titanium trichloride, [1-bis(3,5-dimethylphenyl)methyl-2,3,4-trimethyl-cyclopentadienyl]titanium trichloride, [1-bis(3,5-dimethylphenyl)methyl-2,3,5-trimethyl-cyclopentadienyl]titanium trichloride, [1-bis(3,5-dimethylphenyl)methyl-2,3,4,5-tetramethyl-cyclopentadienyl]titanium trichloride, [1-bis(3,5-dimethylphenyl)methyl-2-ethyl-cyclopentadienyl]titanium trichloride, [1-bis(3,5-dimethylphenyl)methyl-3-ethyl-cyclopentadienyl]titanium trichloride, [1-bis(3,5-dimethylphenyl)methyl-2-n-propyl-cyclopentadienyl]titanium trichloride, [1-bis(3,5-dimethylphenyl)methyl-3-n-propyl-cyclopentadienyl]titanium trichloride, [1-bis(3,5-dimethylphenyl)methyl-2-isopropyl-cyclopentadienyl]titanium trichloride, [1-bis(3,5-dimethylphenyl)methyl-3-isopropyl-cyclopentadienyl]titanium trichloride, [1-bis(3,5-dimethylphenyl)methyl-2-n-butyl-cyclopentadienyl]titanium trichloride, [1-bis(3,5-dimethylphenyl)methyl-3-n-butyl-cyclopentadienyl]titanium trichloride, [1-bis(3,5-dimethylphenyl)methyl-2-sec-butyl-cyclopentadienyl]titanium trichloride, [1-bis(3,5-dimethylphenyl)methyl-3-sec-butyl-cyclopentadienyl]titanium trichloride, [1-bis(3,5-dimethylphenyl)methyl-2-tert-butyl-cyclopentadienyl]titanium trichloride, [1-bis(3,5-dimethylphenyl)methyl-3-tert-butyl-cyclopentadienyl]titanium trichloride, [1-bis(3,5-dimethylphenyl)methyl-2-phenyl-cyclopentadienyl]titanium trichloride, [1-bis(3,5-dimethylphenyl)methyl-3-phenyl-cyclopentadienyl]titanium trichloride, [1-bis(3,5-dimethylphenyl)methyl-2-benzyl-cyclopentadienyl]titanium trichloride, [1-bis(3,5-dimethylphenyl)methyl-3-benzyl-cyclopentadienyl]titanium trichloride, [1-bis(3,5-dimethylphenyl)methyl-indenyl]titanium trichloride, [1-bis(3,5-dimethylphenyl)methyl-2-methylindenyl]titanium trichloride, [9-bis(3,5-dimethylphenyl)methyl-fluorenyl]titanium trichloride, [1-bis(3,5-dimethylphenyl)methyl-tetrahydroindenyl]titanium trichloride, [1-bis(3,5-dimethylphenyl)methyl-2-methyltetrahydroindenyl]titanium trichloride, [9-bis(3,5-dimethylphenyl)methyl-octahydrofluorenyl]titanium trichloride, [3-bis(3,5-dimethylphenyl)methyl-1-trimethylsilyl-cyclopentadienyl]titanium trichloride,

[1-(1,1-diphenyl-ethyl)-cyclopentadienyl]titanium trichloride, [1-(1,1-diphenyl-ethyl)-2-methyl-cyclopentadienyl]titanium trichloride, [1-(1,1-diphenyl-ethyl)-3-methyl-cyclopentadienyl]titanium trichloride, [1-(1,1-diphenyl-ethyl)-2,3-dimethyl-cyclopentadienyl]titanium trichloride, [1-(1,1-diphenyl-ethyl)-2,4-dimethyl-cyclopentadienyl]titanium trichloride, [1-(1,1-diphenyl-ethyl)-3,4-dimethyl-cyclopentadienyl]titanium trichloride, [1-(1,1-diphenyl-ethyl)-2,3,4-trimethyl-cyclopentadienyl]titanium trichloride, [1-(1,1-diphenyl-ethyl)-2,3,5-trimethyl-cyclopentadienyl]titanium trichloride, [1-(1,1-diphenyl-ethyl)-2,3,4,5-tetramethyl-cyclopentadienyl]titanium trichloride, [1-(1,1-diphenyl-ethyl)-2-ethyl-cyclopentadienyl]titanium trichloride, [1-(1,1-diphenyl-ethyl)-3-ethyl-cyclopentadienyl]titanium trichloride, [1-(1,1-diphenyl-ethyl)-2-n-propyl-cyclopentadienyl]titanium trichloride, [1-(1,1-diphenyl-ethyl)-3-n-propyl-cyclopentadienyl]titanium trichloride, [1-(1,1-diphenyl-ethyl)-2-isopropyl-cyclopentadienyl]titanium trichloride, [1-(1,1-diphenyl-ethyl)-3-isopropyl-cyclopentadienyl]titanium trichloride, [1-(1,1-diphenyl-ethyl)-2-n-butyl-cyclopentadienyl]titanium trichloride, [1-(1,1-diphenyl-ethyl)-3-n-butyl-cyclopentadienyl]titanium trichloride, [1-(1,1-diphenyl-ethyl)-2-sec-butyl-cyclopentadienyl]titanium trichloride, [1-(1,1-diphenyl-ethyl)-3-sec-butyl-cyclopentadienyl]titanium trichloride, [1-(1,1-diphenyl-ethyl)-2-tert-butyl-cyclopentadienyl]titanium trichloride, [1-(1,1-diphenyl-ethyl)-3-tert-butyl-cyclopentadienyl]titanium trichloride, [1-(1,1-diphenyl-ethyl)-2-phenyl-cyclopentadienyl]titanium trichloride, [1-(1,1-diphenyl-ethyl)-3-phenyl-cyclopentadienyl]titanium trichloride, [1-(1,1-diphenyl-ethyl)-2-benzyl-cyclopentadienyl]titanium trichloride, [1-(1,1-diphenyl-ethyl)-3-benzyl-cyclopentadienyl]titanium trichloride, [1-(1,1-diphenyl-ethyl)-indenyl]titanium trichloride, [1-(1,1-diphenyl-ethyl)-2-methylindenyl]titanium trichloride, [1-(1,1-diphenyl-ethyl)-tetrahydroindenyl]titanium trichloride, [9-(1,1-diphenyl-ethyl)-fluorenyl]titanium trichloride, [1-(1,1-diphenyl-ethyl)-2-methyltetrahydroindenyl]titanium trichloride, [9-(1,1-diphenyl-ethyl)-octahydrotluorenyl]titanium trichloride, [3-(1,1-diphenyl-ethyl)-1-trimethylsilyl-cyclopentadienyl]titanium trichloride,

[1-(1,1-diphenyl-pentyl)-cyclopentadienyl]titanium trichloride, [1-(1,1-diphenyl-pentyl)-2-methyl-cyclopentadienyl]titanium trichloride, [1-(1,1-diphenyl-pentyl)-3-methyl-cyclopentadienyl]titanium trichloride, [1-(1,1-diphenyl-pentyl)-2,3-dimethyl-cyclopentadienyl]titanium trichloride, [1-(1,1-diphenyl-pentyl)-2,4-dimethyl-cyclopentadienyl]titanium trichloride, [1-(1,1-diphenyl-pentyl)-3,4-dimethyl-cyclopentadienyl]titanium trichloride, [1-(1,1-diphenyl-pentyl)-2,3,4-trimethyl-cyclopentadienyl]titanium trichloride, [1-(1,1-diphenyl-pentyl)-2,3,5-trimethyl-cyclopentadienyl]titanium trichloride, [1-(1,1-diphenyl-pentyl)-2,3,4,5-tetramethyl-cyclopentadienyl]titanium trichloride, [1-(1,1-diphenyl-pentyl)-2-ethyl-cyclopentadienyl]titanium trichloride, [1-(1,1-diphenyl-pentyl)-3-ethyl-cyclopentadienyl]titanium trichloride, [1-(1,1-diphenyl-pentyl)-2-n-propyl-cyclopentadienyl]titanium trichloride, [1-(1,1-diphenyl-pentyl)-3-n-propyl-cyclopentadienyl]titanium trichloride, [1-(1,1-diphenyl-pentyl)-2-isopropyl-cyclopentadienyl]titanium trichloride, [1-(1,1-diphenyl-pentyl)-3-isopropyl-cyclopentadienyl]titanium trichloride, [1-(1,1-diphenyl-pentyl)-2-n-butyl-cyclopentadienyl]titanium trichloride, [1-(1,1-diphenyl-pentyl)-3-n-butyl-cyclopentadienyl]titanium trichloride, [1-(1,1-diphenyl-pentyl)-2-sec-butyl-cyclopentadienyl]titanium trichloride, 1-(1,1-diphenyl-pentyl)-3-sec-butyl-trichloride, [1-(1,1-diphenyl-pentyl)-2-tert-butyl-cyclopentadienyl]titanium trichloride, [1-(1,1-diphenyl-pentyl)-3-tert-butyl-cyclopentadienyl]titanium trichloride, [1-(1,1-diphenyl-pentyl)-2-phenyl-cyclopentadienyl]titanium trichloride, [1-(1,1-diphenyl-pentyl)-3-phenyl-cyclopentadienyl]titanium trichloride, [1-(1,1-diphenyl-pentyl)-2-benzyl-cyclopentadienyl]titanium trichloride, [1-(1,1-diphenyl-pentyl)-3-benzyl-cyclopentadienyl]titanium trichloride, [1-(1,1-diphenyl-pentyl)-indenyl]titanium trichloride, [1-(1,1-diphenyl-pentyl)-2-methylindenyl]titanium trichloride, [1-(1,1-diphenyl-pentyl)-tetrahydroindenyl]titanium trichloride, [9-(1,1-diphenyl-pentyl)-fluorenyl]titanium trichloride, [1-(1,1-diphenyl-pentyl)-2-methyltetrahydroindenyl]titanium trichloride, [9-(1,1-diphenyl-pentyl)-octahydrofluorenyl]titanium trichloride, [3-(1,1-diphenyl-pentyl)-1-trimethylsilyl-cyclopentadienyl]titanium trichloride,

[1-(1,1-diphenyl-1-tert-butyl-methyl)-cyclopentadienyl]titanium trichloride, [1-(1,1-diphenyl-1-tert-butyl-methyl)-2-methyl-cyclopentadienyl]titanium trichloride, [1-(1,1-diphenyl-1-tert-butyl-methyl)-3-methyl-cyclopentadienyl]titanium trichloride, [1-(1,1-diphenyl-1-tert-butyl-methyl)-2,3-dimethyl-cyclopentadienyl]titanium trichloride, [1-(1,1-diphenyl-1-tert-butyl-methyl)-2,4-dimethyl-cyclopentadienyl]titanium trichloride, [1-(1,1-diphenyl-1-tert-butyl-methyl)-3,4-dimethyl-cyclopentadienyl]titanium trichloride, [1-(1,1-diphenyl-1-tert-butyl-methyl)-2,3,4-trimethyl-cyclopentadienyl]titanium trichloride, [1-(1,1-diphenyl-1-tert-butyl-methyl)-2,3,5-trimethyl-cyclopentadienyl]titanium trichloride, [1-(1,1-diphenyl-1-tert-butyl-methyl)-2,3,4,5-tetramethyl-cyclopentadienyl]titanium trichloride, [1-(1,1-diphenyl-1-tert-butyl-methyl)-2-ethyl-cyclopentadienyl]titanium trichloride, [1-(1,1-diphenyl-1-tertbutyl-methyl)-3-ethyl-cyclopentadienyl]titanium trichloride, [1-(1,1-diphenyl-1-tert-butyl-methyl)-2-n-propyl-cyclopentadienyl]titanium trichloride, [1-(1,1-diphenyl-1-tert-butyl-methyl)-3-n-propyl-cyclopentadienyl]titanium trichloride, [1-(1,1-diphenyl-1-tert-butyl-methyl)-2-isopropyl-cyclopentadienyl]titanium trichloride, [1-(1,1-diphenyl-1-tert-butyl-methyl)-3-isopropyl-cyclopentadienyl]titanium trichloride, [1-(1,1-diphenyl-1-tert-butyl-methyl)-2-n-butyl-cyclopentadienyl]titanium trichloride, [1-(1,1-diphenyl-1-tert-butyl-methyl)-3-n-butyl-cyclopentadienyl]titanium trichloride, [1-(1,1-diphenyl-1-tert-butyl-methyl)-2-sec-butyl-cyclopentadienyl]titanium trichloride, [1-(1,1-diphenyl-1-tert-butyl-methyl)-3-sec-butyl-cyclopentadienyl]titanium trichloride, [1-(1,1-diphenyl-1-tert-butyl-methyl)-2-tert-butyl-cyclopentadienyl]titanium trichloride, [1-(1,1-diphenyl-1-tert-butyl-methyl)-3-tert-butyl-cyclopentadienyl]titanium trichloride, [1-(1,1-diphenyl-1-tert-butyl-methyl)-2-phenyl-cyclopentadienyl]titanium trichloride, [1-(1,1-diphenyl-1-tert-butyl-methyl)-3-phenyl-cyclopentadienyl]titanium trichloride, [1-(1,1-diphenyl-1-tert-butyl-methyl)-2-benzyl-cyclopentadienyl]titanium trichloride, [1-(1,1-diphenyl-1-tert-butyl-methyl)-3-benzyl-cyclopentadienyl]titanium trichloride, [1-(1,1-diphenyl-1-tert-butyl-methyl)-indenyl]titanium trichloride, [1-(1,1-diphenyl-1-tert-butyl-methyl)-2-methylindenyl]titanium trichloride, [1-(1,1-diphenyl-1-tert-butyl-methyl)-tetrahydroindenyl]titanium trichloride, [9-(1,1-diphenyl-1-tert-butyl-methyl)-fluorenyl]titanium trichloride, [1-(1,1-diphenyl-1-tert-butyl-methyl)-2-methyltetrahydroindenyl]titanium trichloride, [9-(1,1-diphenyl-1-tert-butyl-methyl)-octahydrofluorenyl]titanium trichloride and [3-(1,1-diphenyl-1-tert-butyl-methyl)-1-trimethylsilyl-cyclopentadienyl]titanium trichloride.

Furthermore, examples of the transition metal complex (1-2) wherein J is a carbon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Examples of the transition metal complex (1-3) wherein J is a silicon atom include the following complexes:

[1-triphenylsilyl-2,3,4,5-tetramethylcyclopentadienyl]titanium trichloride, [1-phenyldi(2-methylphenyl)silyl-2,3,4,5-tetramethylcyclopentadienyl]titanium trichloride, [1-phenyldi(3-methylphenyl)silyl-2,3,4,5-tetramethylcyclopentadienyl]titanium trichloride, [1-phenyldi(4-methylphenyl)silyl-2,3,4,5-tetramethylcyclopentadienyl]titanium trichloride, [1-phenylbis(2,3-dimethylphenyl)silyl-2,3,4,5-tetramethylcyclopentadienyl]titanium trichloride, [1-phenylbis(2,4-dimethylphenyl)silyl-2,3,4,5-tetramethylcyclopentadienyl]titanium trichloride, [1-phenylbis(2,5-dimethylphenyl)silyl-2,3,4,5-tetramethylcyclopentadienyl]titanium trichloride, [1-phenylbis(2,6-dimethylphenyl)silyl-2,3,4,5-tetramethylcyclopentadienyl]titanium trichloride, [1-phenylbis(3,5-dimethylphenyl)silyl-2,3,4,5-tetramethyl cyclopentadienyl]titanium trichloride, [1-phenylbis(3,4,5-trimethylphenyl)silyl-2,3,4,5-tetramethylcyclopentadienyl]titanium trichloride,

[1-diphenyl(2-methylphenyl)silyl-2,3,4,5-tetramethylcyclopentadienyl]titanium trichloride, [1-diphenyl(3-methylphenyl)silyl-2,3,4,5-tetramethylcyclopentadienyl]titanium trichloride, [1-diphenyl(4-methylphenyl)silyl-2,3,4,5-tetramethylcyclopentadienyl]titanium trichloride, [1-diphenyl(2,3-dimethylphenyl)silyl-2,3,4,5-tetramethylcyclopentadienyl]titanium trichloride, [1-diphenyl(2,4-dimethylphenyl)silyl-2,3,4,5-tetramethylcyclopentadienyl]titanium trichloride, [1-diphenyl(2,5-dimethylphenyl)silyl-2,3,4,5-tetramethylcyclopentadienyl]titanium trichloride, [1-diphenyl(2,6-dimethylphenyl)silyl-2,3,4,5-tetramethylcyclopentadienyl]titanium trichloride, [1-diphenyl(3,5-dimethylphenyl)silyl-2,3,4,5-tetramethylcyclopentadienyl]titanium trichloride, [1-diphenyl(3,4,5-trimethylphenyl)silyl-2,3,4,5-tetramethylcyclopentadienyl]titanium trichloride,

[1-phenyl(2-methylphenyl)(3,5-dimethylphenyl)silyl-2,3,4,5-tetramethylcyclopentadienyl]titanium trichloride, [1-phenyl(3-methylphenyl)(3,5-dimethylphenyl)silyl-2,3,4,5-tetramethylcyclopentadienyl]titanium trichloride, [1-phenyl(4-methylphenyl)(3,5-dimethylphenyl)silyl-2,3,4,5-tetramethylcyclopentadienyl]titanium trichloride, [1-phenyl(2,3-dimethylphenyl)(3,5-dimethylphenyl)silyl-2,3,4,5-tetramethylcyclopentadienyl]titanium trichloride, [1-phenyl(2,4-dimethylphenyl)(3,5-dimethylphenyl)silyl-2,3,4,5-tetramethyl cyclopentadienyl]titanium trichloride, [1-phenyl(2,5-dimethylphenyl)(3,5-dimethylphenyl)silyl-2,3,4,5-tetramethylcyclopentadienyl]titanium trichloride, [1-phenyl(2,6-dimethylphenyl)(3,5-dimethylphenyl)silyl-2,3,4,5-tetramethylcyclopentadienyl]titanium trichloride, [1-phenyl(3,5-dimethylphenyl)(3,4,5-trimethylphenyl)silyl-2,3,4,5-tetramethylcyclopentadienyl]titanium trichloride,

[1-di(2-methylphenyl)(3,5-dimethylphenyl)silyl-2,3,4,5-tetramethylcyclopentadienyl]titanium trichloride, [1-di(3-methylphenyl)(3,5-dimethylphenyl)silyl-2,3,4,5-tetramethylcyclopentadienyl]titanium trichloride, [1-di(4-methylphenyl)(3,5-dimethylphenyl)silyl-2,3,4,5-tetramethylcyclopentadienyl]titanium trichloride, [1-bis(2,3-dimethylphenyl)(3,5-dimethylphenyl)silyl-2,3,4,5-tetramethylcyclopentadienyl]titanium trichloride, [1-bis(2,4-dimethylphenyl)(3,5-dimethylphenyl)silyl-2,3,4,5-tetramethylcyclopentadienyl]titanium trichloride, [1-bis(2,5-dimethylphenyl)(3,5-dimethylphenyl)silyl-2,3,4,5-tetramethylcyclopentadienyl]titanium trichloride, [1-bis(2,6-dimethylphenyl)(3,5-dimethylphenyl)silyl-2,3,4,5-tetramethylcyclopentadienyl]titanium trichloride, [1-tris(3,5-dimethylphenyl)silyl-2,3,4,5-tetramethylcyclopentadienyl]titanium trichloride and [1-(3,5-dimethylphenyl)bis(3,4,5-dimethylphenyl)silyl-2,3,4,5-tetramethylcyclopentadienyl]titanium trichloride.

Moreover, examples of the transition metal complex (1-3) wherein J is a silicon atom also include titanium chloride complexes obtained by substituting “cyclopentadienyl”, “2-methylcyclopentadienyl”, “3-methylcyclopentadienyl”, “2,3-dimethylcyclopentadienyl”, “2,4-dimethylcyclopentadienyl”, “2,5-dimethylcyclopentadienyl”, “2,3,5-trimethylcyclopentadienyl”, “2-ethylcyclopentadienyl”, “3-ethylcyclopentadienyl”, “2-n-propylcyclopentadienyl”, “3-n-propylcyclopentadienyl”, “2-isopropylcyclopentadienyl”, “3-isopropylcyclopentadienyl”, “2-n-butylcyclopentadienyl”, “3-n-butylcyclopentadienyl”, “2-sec-butylcyclopentadienyl”, “3-sec-butylcyclopentadienyl”, “2-tert-butylcyclopentadienyl”, “3-tert-butylcyclopentadienyl”, “2-phenylcyclopentadienyl”, “3-phenylcyclopentadienyl”, “2-benzylcyclopentadienyl”, “3-benzylcyclopentadienyl”, “indenyl”, “2-methylindenyl”, “fluorenyl”, “tetrahydroindenyl”, “2-methyltetrahydroindenyl” or “octahydrofluorenyl” for “2,3,4,5-tetramethylcyclopentadienyl” in the titanium chloride complexes exemplified above.

Furthermore, examples of the transition metal complex (1-3) wherein J is a silicon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Examples of the transition metal complex (1-3) wherein J is a carbon atom include the following complexes:

[1-triphenylmethyl-cyclopentadienyl]titanium trichloride, [1-triphenylmethyl-2-methyl-cyclopentadienyl]titanium trichloride, [1-triphenylmethyl-3-methyl-cyclopentadienyl]titanium trichloride, [1-triphenylmethyl-2,3-dimethyl-cyclopentadienyl]titanium trichloride, [1-triphenylmethyl-2,4-dimethyl-cyclopentadienyl]titanium trichloride, [1-triphenylmethyl-3,4-dimethyl-cyclopentadienyl]titanium trichloride, [1-triphenylmethyl-2,3,4-trimethyl-cyclopentadienyl]titanium trichloride, [1-triphenylmethyl-2,3,5-trimethyl-cyclopentadienyl]titanium trichloride, [1-triphenylmethyl-2,3,4,5-tetramethyl-cyclopentadienyl]titanium trichloride, [1-triphenylmethyl-2-ethyl-cyclopentadienyl]titanium trichloride, [1-triphenylmethyl-3-ethyl-cyclopentadienyl]titanium trichloride, [1-triphenylmethyl-2-n-propyl-cyclopentadienyl]titanium trichloride, [1-triphenylmethyl-3-n-propyl-cyclopentadienyl]titanium trichloride, [1-triphenylmethyl-2-isopropyl-cyclopentadienyl]titanium trichloride, [1-triphenylmethyl-3-isopropyl-cyclopentadienyl]titanium trichloride, [1-triphenylmethyl-2-n-butyl-cyclopentadienyl]titanium trichloride, [1-triphenylmethyl-3-n-butyl-cyclopentadienyl]titanium trichloride, [1-triphenylmethyl-2-sec-butyl-cyclopentadienyl]titanium trichloride, [1-triphenylmethyl-3-sec-butyl-cyclopentadienyl]titanium trichloride, [1-triphenylmethyl-2-tort-butyl-cyclopentadienyl]titanium trichloride, [1-triphenylmethyl-3-tert-butyl-cyclopentadienyl]titanium trichloride, [1-triphenylmethyl-2-phenyl-cyclopentadienyl]titanium trichloride, [1-triphenylmethyl-3-phenyl-cyclopentadienyl]titanium trichloride, [1-triphenylmethyl-2-benzyl-cyclopentadienyl]titanium trichloride, [1-triphenylmethyl-3-benzyl-cyclopentadienyl]titanium trichloride, [1-triphenylmethyl-indenyl]titanium trichloride, [1-triphenylmethyl-2-methylindenyl]titanium trichloride, [1-triphenylmethyl-tetrahydroindenyl]titanium trichloride, [9-triphenylmethyl-fluorenyl]titanium trichloride, [1-triphenylmethyl-2-methyltetrahydroindenyl]titanium trichloride, [9-triphenylmethyl-octahydrofluorenyl]titanium trichloride, [1-trimethylsilyl-3-triphenylmethyl-cyclopentadienyl]titanium trichloride, [1-triethylsilyl-3-triphenylmethyl-cyclopentadienyl]titanium trichloride, [1-triisopropylsilyl-3-triphenylmethyl-cyclopentadienyl]titanium trichloride, [1-tert-butyldimethylsilyl-3-triphenylmethyl-cyclopentadienyl]titanium trichloride,

[1-tri(3-methylphenyl)methyl-cyclopentadienyl]titanium trichloride, [1-tri(3-methylphenyl)methyl-2-methyl-cyclopentadienyl]titanium trichloride, [1-tri(3-methylphenyl)methyl-3-methyl-cyclopentadienyl]titanium trichloride, [1-tri(3-methylphenyl)methyl-2,3-dimethyl-cyclopentadienyl]titanium trichloride, [1-tri(3-methylphenyl)methyl-2,4-dimethyl-cyclopentadienyl]titanium trichloride, [1-tri(3-methylphenyl)methyl-3,4-dimethyl-cyclopentadienyl]titanium trichloride, [1-tri(3-methylphenyl)methyl-2,3,4-trimethyl-cyclopentadienyl]titanium trichloride, [1-tri(3-methylphenyl)methyl-2,3,5-trimethyl-cyclopentadienyl]titanium trichloride, [1-tri(3-methylphenyl)methyl-2,3,4,5-tetramethyl-cyclopentadienyl]titanium trichloride, [1-tri(3-methylphenyl)methyl-2-ethyl-cyclopentadienyl]titanium trichloride, [1-tri(3-methylphenyl)methyl-3-ethyl-cyclopentadienyl]titanium trichloride, [1-tri(3-methylphenyl)methyl-2-n-propyl-cyclopentadienyl]titanium trichloride, [1-tri(3-methylphenyl)methyl-3-n-propyl-cyclopentadienyl]titanium trichloride, [1-tri(3-methylphenyl)methyl-2-isopropyl-cyclopentadienyl]titanium trichloride, [1-tri(3-methylphenyl)methyl-3-isopropyl-cyclopentadienyl]titanium trichloride, [1-tri(3-methylphenyl)methyl-2-n-butyl-cyclopentadienyl]titanium trichloride, [1-tri(3-methylphenyl)methyl-3-n-butyl-cyclopentadienyl]titanium trichloride, [1-tri(3-methylphenyl)methyl-2-sec-butyl-cyclopentadienyl]titanium trichloride, [1-tri(3-methylphenyl)methyl-3-sec-butyl-cyclopentadienyl]titanium trichloride, [1-tri(3-methylphenyl)methyl-2-tert-butyl-cyclopentadienyl]titanium trichloride, [1-tri(3-methylphenyl)methyl-3-tert-butyl-cyclopentadienyl]titanium trichloride, [1-tri(3-methylphenyl)methyl-2-phenyl-cyclopentadienyl]titanium trichloride, [1-tri(3-methylphenyl)methyl-3-phenyl-cyclopentadienyl]titanium trichloride, [1-tri(3-methylphenyl)methyl-2-benzyl-cyclopentadienyl]titanium trichloride, [1-tri(3-methylphenyl)methyl-3-benzyl-cyclopentadienyl]titanium trichloride, [1-tri(3-methylphenyl)methyl-indenyl]titanium trichloride, [1-tri(3-methylphenyl)methyl-2-methylindenyl]titanium trichloride, [9-tri(3-methylphenyl)methyl-fluorenyl]titanium trichloride, [1-tri(3-methylphenyl)methyl-tetrahydroindenyl]titanium trichloride, [1-tri(3-methylphenyl)methyl-2-methyltetrahydroindenyl]titanium trichloride, [9-tri(3-methylphenyl)methyl-octahydrofluorenyl]titanium trichloride, [1-trimethylsilyl-3-tri(3-methylphenyl)methyl-cyclopentadienyl]titanium trichloride, [1-triethylsilyl-3-tri(3-methylphenyl)methyl-cyclopentadienyl]titanium trichloride, [1-triisopropyl silyl-3-tri(3-methylphenyl)methyl-cyclopentadienyl]titanium trichloride, [1-tert-butyldimethylsilyl-3-tri(3-methylphenyl)methyl-cyclopentadienyl]titanium trichloride,

[1-tris(3,5-dimethylphenyl)methyl-cyclopentadienyl]titanium trichloride, [1-tris(3,5-dimethylphenyl)methyl-2-methyl-cyclopentadienyl]titanium trichloride, [1-tris(3,5-dimethylphenyl)methyl-3-methyl-cyclopentadienyl]titanium trichloride, [1-tris(3,5-dimethylphenyl)methyl-2,3-dimethyl-cyclopentadienyl]titanium trichloride, [1-tris(3,5-dimethylphenyl)methyl-2,4-dimethyl-cyclopentadienyl]titanium trichloride, [1-tris(3,5-dimethylphenyl)methyl-3,4-dimethyl-cyclopentadienyl]titanium trichloride, [1-tris(3,5-dimethylphenyl)methyl-2,3,4-trimethyl-cyclopentadienyl]titanium trichloride, [1-tris(3,5-dimethylphenyl)methyl-2,3,5-trimethyl-cyclopentadienyl]titanium trichloride, [1-tris(3,5-dimethylphenyl)methyl-2,3,4,5-tetramethyl-cyclopentadienyl]titanium trichloride, [1-tris(3,5-dimethylphenyl)methyl-2-ethyl-cyclopentadienyl]titanium trichloride, [1-tris(3,5-dimethylphenyl)methyl-3-ethyl-cyclopentadienyl]titanium trichloride, [1-tris(3,5-dimethylphenyl)methyl-2-n-propyl-cyclopentadienyl]titanium trichloride, [1-tris(3,5-dimethylphenyl)methyl-3-n-propyl-cyclopentadienyl]titanium trichloride, [1-tris(3,5-dimethylphenyl)methyl-2-isopropyl-cyclopentadienyl]titanium trichloride, [1-tris(3,5-dimethylphenyl)methyl-3-isopropyl-cyclopentadienyl]titanium trichloride, [1-tris(3,5-dimethylphenyl)methyl-2-n-butyl-cyclopentadienyl]titanium trichloride, [1-tris(3,5-dimethylphenyl)methyl-3-n-butyl-cyclopentadienyl]titanium trichloride, [1-tris(3,5-dimethylphenyl)methyl-2-sec-butyl-cyclopentadienyl]titanium trichloride, [1-tris(3,5-dimethylphenyl)methyl-3-sec-butyl-cyclopentadienyl]titanium trichloride, [1-tris(3,5-dimethylphenyl)methyl-2-tert-butyl-cyclopentadienyl]titanium trichloride, [1-tris(3,5-dimethylphenyl)methyl-3-tert-butyl-cyclopentadienyl]titanium trichloride, [1-tris(3,5-dimethylphenyl)methyl-2-phenyl-cyclopentadienyl]titanium trichloride, [1-tris(3,5-dimethylphenyl)methyl-3-phenyl-cyclopentadienyl]titanium trichloride, [1-tris(3,5-dimethylphenyl)methyl-2-benzyl-cyclopentadienyl]titanium trichloride, [1-tris(3,5-dimethylphenyl)methyl-3-benzyl-cyclopentadienyl]titanium trichloride, [1-tris(3,5-dimethylphenyl)methyl-indenyl]titanium trichloride, [1-tris(3,5-dimethylphenyl)methyl-2-methylindenyl]titanium trichloride, [9-tris(3,5-dimethylphenyl)methyl-fluorenyl]titanium trichloride, [1-tris(3,5-dimethylphenyl)methyl-tetrahydroindenyl]titanium trichloride, [1-tris(3,5-dimethylphenyl)methyl-2-methyltetrahydroindenyl]titanium trichloride, [9-tris(3,5-dimethylphenyl)methyl-octahydrofluorenyl]titanium trichloride, [1-trimethylsilyl-3-tris(3,5-dimethylphenyl)methyl-cyclopentadienyl]titanium trichloride, [1-triethylsilyl-3-tris(3,5-dimethylphenyl)methyl-cyclopentadienyl]titanium trichloride, [1-triisopropylsilyl-3-tris(3,5-dimethylphenyl)methyl-cyclopentadienyl]titanium trichloride and [1-tert-butyldimethyl silyl-3-tris(3,5-dimethylphenyl)methyl-cyclopentadienyl]titanium trichloride.

Furthermore, examples of the transition metal complex (1-3) wherein J is a carbon atom also include: transition metal chloride complexes such as zirconium chloride complexes obtained by substituting “zirconium” for “titanium” in the complexes exemplified above, or hafnium chloride complexes obtained by substituting “hafnium” therefor; titanium halide complexes such as titanium fluoride complexes obtained by substituting “fluoride” for “chloride” in the complexes, titanium bromide complexes obtained by substituting “bromide” therefor and titanium iodide complexes obtained by substituting “iodide” therefor; titanium hydride complexes obtained by substituting “hydride” therefor; alkylated titanium complexes such as methylated titanium complexes obtained by substituting “methyl” therefor; arylated titanium complexes such as phenylated titanium complexes obtained by substituting “phenyl” therefor; aralkylated titanium complexes such as benzylated titanium complexes obtained by substituting “benzyl” therefor; titanium alkoxide complexes such as titanium methoxide complexes obtained by substituting “methoxide” therefor, titanium n-butoxide complexes obtained by substituting “n-butoxide” therefor and titanium isopropoxide complexes obtained by substituting “isopropoxide” therefor; titanium aryloxide complexes such as titanium phenoxide complexes obtained by substituting “phenoxide” therefor; titanium aralkyloxide complexes such as titanium benzyloxide complexes obtained by substituting “benzyloxide” therefor; and titanium amide complexes such as titanium dimethylamide complexes obtained by substituting “dimethylamide” therefor and titanium diethylamide complexes obtained by substituting “diethylamide” therefor.

Preferable examples of the transition metal complexes of the formulae (1-1) to (1-3) include [1-dimethylphenylsilyl-2,3,4,5-tetramethylcyclopentadienyl]titanium trichloride, [1-methyldiphenylsilyl-2,3,4,5-tetramethylcyclopentadienyl]titanium trichloride, [1-triphenylsilyl-2,3,4,5-tetramethylcyclopentadienyl]titanium trichloride, [1-tris(3,5-dimethylphenyl)silyl-2,3,4,5-tetramethylcyclopentadienyl]titanium trichloride, [1-dimethylphenylsilyl-2,3,4,5-tetramethylcyclopentadienyl]trimethyl titanium, [1-methyldiphenylsilyl-2,3,4,5-tetramethylcyclopentadienyl]trimethyl titanium, [1-triphenylsilyl-2,3,4,5-tetramethylcyclopentadienyl]titanium trichloride, [1-tris(3,5-dimethylphenyl)silyl-2,3,4,5-tetramethylcyclopentadienyl]trimethyl titanium, [1-(1-methyl-1-phenyl)ethyl)-cyclopentadienyl]titanium trichloride, [1-(1-methyl-1-(3,5-dimethylphenyl)ethyl)-3-trimethylsilylcyclopentadienyl]titanium trichloride, [3-diphenylmethyl-1-trimethylsilyl-cyclopentadienyl]titanium trichloride, [3-bis(3,5-dimethylphenyl)methyl-1-trimethylsilyl-cyclopentadienyl]titanium trichloride and [1-trimethylsilyl-3-triphenylmethyl-cyclopentadienyl]titanium trichloride

Examples of the compound (A) used in step 1 include the following compound (A):

(A): an organic aluminum compound represented by formula (E¹)ₐAl(G)₃₋ₐ, wherein E¹ represents a hydrocarbyl group having 2 to 8 carbon atoms; G represents a hydrogen atom or a halogen atom; a represents an integer of 1 to 3; in the case where more than one E¹ groups exist, the E¹ groups may be the same as or different from each other; and in the case where more than one G groups exist, the G groups may be the same as or different from each other.

In the compound (A), examples of the hydrocarbyl group having 2 to 8 carbon atoms in E¹ include alkyl having 2 to 8 carbon atoms. Examples of the alkyl groups having 2 to 8 carbon atoms include ethyl, n-propyl, isopropyl, n-butyl, isobutyl, n-pentyl, neopentyl, n-hexyl, n-heptyl and n-octyl groups. Among them, alkyl groups having 2 to 6 carbon atoms are preferable.

Examples of the organic aluminum compound (A) represented by (E¹)ₐAl(G)₃₋ₐ, include trialkylaluminum, dialkylaluminum chloride, alkylaluminum dichloride and dialkylaluminum hydride. Examples of the trialkylaluminum include triethylaluminum, tripropylaluminum, triisobutylaluminum and trihexylaluminum. Examples of the dialkylaluminum chloride include diethylaluminum chloride, dipropylaluminum chloride, diisobutylaluminum chloride and dihexylaluminum chloride. Examples of the alkylaluminum dichloride include ethylaluminum dichloride, propylaluminum dichloride, isobutylaluminum dichloride and hexylaluminum dichloride. Examples of the dialkylaluminum hydride include diethylaluminum hydride, dipropylaluminum hydride, diisobutylaluminum hydride and dihexylaluminum hydride.

Of the organic aluminum compounds exemplified above, triisobutylaluminum and diisobutylaluminum hydride are preferable, and triisobutylaluminum is more preferable.

Step 1 is the step of obtaining a catalytic component by bringing the transition metal complex (1-1), (1-2) or (1-3) into contact with the compound (A) in the absence of ethylene. A method for bringing them into contact with each other is not particularly limited. They are preferably brought into contact with each other in a solvent. Moreover, when a solvent is used, the compound (A) is mixed with the solvent and then, the transition metal complex (1-1), (1-2) or (1-3) can be added thereto and thereby brought into contact with each other. Alternatively, the transition metal complex (1-1), (1-2) or (1-3) is mixed with the solvent and then, the compound (A) can be added thereto and thereby brought into contact with each other.

The solvent is not particularly limited and, for example, aliphatic hydrocarbyl (e.g., butane, pentane, hexane, heptane and octane), aromatic hydrocarbyl (e.g., benzene and toluene) or halogenated hydrocarbyl (e.g., methylene dichloride and chlorobenzene) can be used.

Regarding the amount of the compound (A) to be used, the molar ratio between the compound (A) and the transition metal complex (compound (A)/transition metal complex) is usually 0.01 to 10000, preferably 5 to 5000.

For use in a solution state, the concentration of the transition metal complex is usually 0.0001 to 5 mmol/L, preferably 0.001 to 1 mmol/L. The concentration of the compound (A) is usually 0.01 to 500 mmol/L, preferably 0.1 to 100 mmol/L.

The catalytic component obtainable in step 1 may be isolated from the solvent and subjected to step 2 or may be subjected to step 2 in the form of a solution containing the catalytic component dissolved therein.

For reducing the amount of by-product polymers, step 1 is preferably performed in the absence of ethylene.

### (Step 2)

Compound B used in the step 2 is one or more compounds selected from the group consisting of the following (B 1), (B2) and (B3):

(B 1): a boron compound represented by formula BQ¹Q²Q³,

(B2): a borate compound represented by formula T⁺(BQ⁴Q⁵Q⁶Q⁷)⁻, and

(B3): a borate compound represented by formula (L-H)⁺(BQ⁸Q⁹Q¹⁰Q¹¹)⁻, wherein

B represents a trivalent boron; Q¹, Q², Q³, Q⁴, Q⁵, Q⁶, Q⁷, Q⁸, Q⁹, Q¹⁰ and Q¹¹ are the same as or different from each other and each represent a halogen atom, a hydrocarbyl group having 1 to 20 carbon atoms which may have a halogen atom as a substituent, a hydrocarbylsilyl group having 1 to 20 carbon atoms which may have a halogen atom as a substituent, an alkoxy group having 1 to 20 carbon atoms which may have a halogen atom as a substituent or a dihydrocarbylamino group having 2 to 20 carbon atoms which may have a halogen atom as a substituent; T⁺ represents an inorganic or organic cation; and (L-H)⁺ represents a Broensted acid.

In the compounds (B1) to (B3), Q¹, Q², Q³, Q⁴, Q⁵, Q⁶, Q⁷, Q⁸, Q⁹, Q¹⁰ and Q¹¹ are preferably a halogen atom or a hydrocarbyl group having 1 to 20 carbon atoms which may have a halogen atom as a substituent. Examples of the inorganic cation represented by T⁺ include a ferrocenium cation, an alkyl-substituted ferrocenium cation, and a silver cation. Examples of the organic cation include a triphenylmethyl cation. Examples of (BQ⁴Q⁵Q⁶Q⁷)⁻ and (BQ⁸Q⁹Q¹⁰Q¹¹)⁻ include tetrakis(pentafluorophenyl)borate, tetrakis(2,3,5,6-tetrafluorophenyl)borate, tetrakis(2,3,4,5-tetrafluorophenyl)borate, tetrakis(3,4,5-trifluorophenyl)borate, tetrakis(2,3,4-trifluorophenyl)borate, phenyltris(pentafluorophenyl)borate, and tetrakis(3,5-bistrifluoromethylphenyl)borate. Examples of the Broensted acid represented by (L-H)⁺ include trialkyl-substituted ammonium, N,N-dialkylanilinium, dialkylammonium and triarylphosphonium.

Examples of the boron compound (B1) represented by formula BQ¹Q²Q³ include tris(pentafluorophenyl)borane, tris(2,3,5,6-tetrafluorophenyl)borane, tris(2,3,4,5-tetrafluorophenyl)borane, tris(3,4,5-trifluorophenyl)borane, tris(2,3,4-trifluorophenyl)borane, and phenylbis(pentafluorophenyl)borane.

Examples of the borate compound (B2) represented by formula T⁺(BQ⁴Q⁵Q⁶Q⁷)⁻ include ferrocenium tetrakis(pentafluorophenyl)borate, 1,1′-bis trimethylsilylferrocenium tetrakis(pentafluorophenyl)borate, silver tetrakis(pentafluorophenyl)borate, triphenylmethyl tetrakis(pentafluorophenyl)borate and triphenylmethyl tetrakis(3,5-bistrifluoromethylphenyl)borate.

Examples of the borate compound (B3) represented by formula (L-H)⁺(BQ⁸Q⁹Q¹⁰Q¹¹)− include triethylammonium tetrakis(pentafluorophenyl)borate, tripropylammonium tetrakis(pentafluorophenyl)borate, tri(normal butyl)ammonium tetrakis(pentafluorophenyl)borate, tri(normal butyl)ammonium tetrakis(3,5-bistrifluoromethylphenyl)borate, N,N-dimethylanilinium tetrakis(pentafluorophenyl)borate, N,N-bis-trimethylsilylanilinium tetrakis(pentafluorophenyl)borate, N,N-diethylanilinium tetrakis(pentafluorophenyl)borate, N,N-2,4,6-pentamethylanilinium tetrakis(pentafluorophenyl)borate, N,N-bis-trimethylsilylanilinium tetrakis(3,5-bistrifluoromethylphenyl)borate, diisopropylammonium tetrakis(pentafluorophenyl)borate, dicyclohexylammonium tetrakis(pentafluorophenyl)borate, triphenylphosphonium tetrakis(pentafluorophenyl)borate, tri(methylphenyl)phosphonium tetrakis(pentafluorophenyl)borate, tri(bis-trimethylsilylphenyl)phosphonium tetrakis(pentafluorophenyl)borate and tri(dimethylphenyl)phosphonium tetrakis(pentafluorophenyl)borate.

In step 2, the method for bringing the catalytic component into contact with the compound (B) is not particularly limited. The catalytic component is preferably brought into contact with the compound (B) in the presence of a solvent. Moreover, when a solvent is used, the catalytic component is mixed with the solvent and then, the compound (B) can be added thereto and thereby brought into contact with the catalytic component.

The molar ratio between the compound (B) and the transition metal complex (compound (B)/transition metal complex) is usually 0.01 to 100, preferably 0.5 to 10.

The concentration of the compound (B) is usually 0.001 to 50 mmol/L, preferably 0.01 to 20 mmol/L.

The solvent is not particularly limited and, for example, aliphatic hydrocarbyl (e.g., butane, pentane, hexane, heptane and octane), aromatic hydrocarbyl (e.g., benzene and toluene) or halogenated hydrocarbyl (e.g., methylene dichloride and chlorobenzene) can be used.

Trimerization reaction is performed in the presence of a catalyst obtainable by bringing the catalytic component into contact with the compound (B). The trimerization reaction may be, for example, trimerization reaction using aliphatic hydrocarbyl (e.g., butane, pentane, hexane, heptane and octane), aromatic hydrocarbyl (e.g., benzene and toluene) or halogenated hydrocarbyl (e.g., methylene dichloride and chlorobenzene) as a solvent, trimerization reaction in a slurry state, or trimerizing gaseous ethylene.

The trimerization reaction can be performed by any of batch, semi-continuous and continuous methods.

The pressure of ethylene in the trimerization reaction is usually normal pressure to 10 MPa, preferably in the range of normal pressure to 5 MPa.

The temperature of the trimerization reaction can usually be in the range of −50° C. to 220° C. and is preferably in the range of 0° C. to 170° C., more preferably in the range of 50° C. to 120° C.

The time of the trimerization reaction can generally be determined appropriately depending on the reaction apparatus of interest and can be in the range of 1 minute to 20 hours.

### <Methods for Producing Transition Metal Complexes (1-1) to (1-3)>

The transition metal complexes (1-1), (1-2) and (1-3) wherein J is a silicon atom can be produced from a substituted cyclopentadiene compound represented by formula (7-1) (hereinafter, abbreviated to a “substituted cyclopentadiene compound (7-1)”), a substituted cyclopentadiene compound represented by formula (7-2) (hereinafter, abbreviated to a “substituted cyclopentadiene compound (7-2)”) and a substituted cyclopentadiene compound represented by formula (7-3) (hereinafter, abbreviated to a “substituted cyclopentadiene compound (7-3)”), respectively, by similar methods:

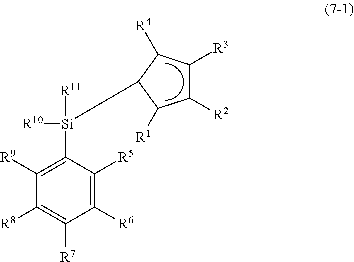

wherein R¹, R², R³, R⁴, R⁵, R⁶, R⁷, R⁸, R⁹, R¹⁰ and R¹¹ are as defined above,

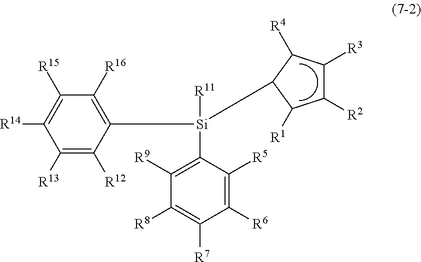

wherein R¹, R², R³, R⁴, R⁵, R⁶, R⁷, R⁸, R⁹, R¹¹, R¹², R¹³, R¹⁴, R¹⁵ and R¹⁶ are as defined above, and

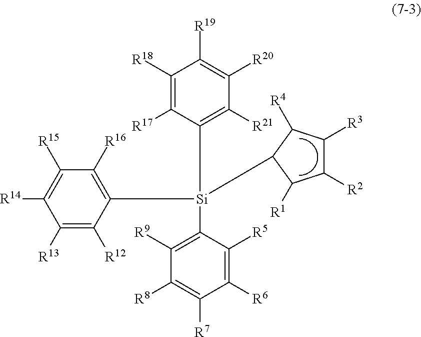

wherein R¹, R², R³, R⁴, R⁵, R⁶, R⁷, R⁸, R⁹, R¹², R¹³, R¹⁴, R¹⁵, R¹⁶, R¹⁷, R¹⁸, R¹⁹, R²⁰, and R²¹ are as defined above.

The transition metal complexes (1-1), (1-2) and (1-3) wherein J is a carbon atom can be produced from a substituted cyclopentadiene compound represented by formula (7-4) (hereinafter, abbreviated to a “substituted cyclopentadiene compound (7-4)”), a substituted cyclopentadiene compound represented by formula (7.5) (hereinafter, abbreviated to a “substituted cyclopentadiene compound (7-5)”) and a substituted cyclopentadiene compound represented by formula (7-6) (hereinafter, abbreviated to a “substituted cyclopentadiene compound (7-6)”), respectively, by similar methods:

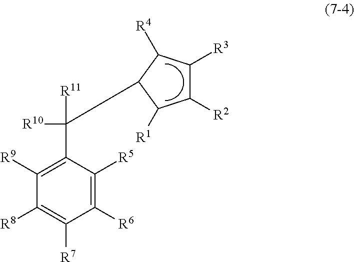

wherein R¹, R², R³, R⁴, R⁵, R⁶, R⁷, R⁸, R⁹, R¹⁰ and R¹¹ are as defined above,

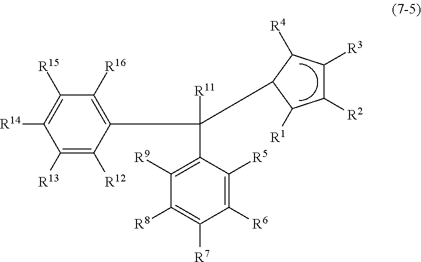

wherein R¹, R², R³, R⁴, R⁵, R⁶, R⁷, R⁸, R⁹, R¹⁰, R¹¹, R¹², R¹³, R¹⁴, R¹⁵ and R¹⁶ are as defined above, and

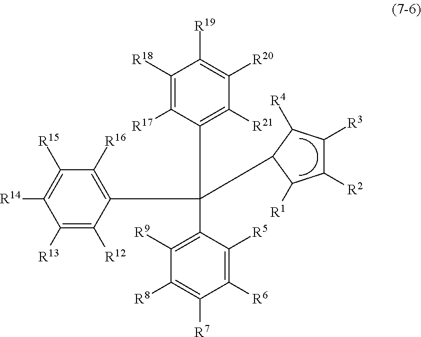

wherein R¹, R², R³, R⁴, R⁵, R⁶, R⁷, R⁸, R⁹, R¹², R¹³, R¹⁴, R¹⁵, R¹⁶, R¹⁷, R¹⁸, R¹⁹, R²⁰, and R²¹ are as defined above.

The transition metal complex (1-1) wherein J is a carbon atom can be produced by, for example, a method described in Organometallics 2002, 21, 5122-5135.

Hereinafter, a method for producing the transition metal complex (1-3) wherein J is a silicon atom will be described as an example.

The transition metal complexes (1-1) and (1-2) wherein J is a silicon atom and the transition metal complexes (1-1), (1-2) and (1-3) wherein J is a carbon atom can also be produced similarly thereto.

The transition metal complex (1-3) can be produced by, for example, a production method comprising the steps of:

reacting the substituted cyclopentadiene compound (7-3) with a base in the presence of an amine compound; and
reacting the reaction product of the substituted cyclopentadiene compound (7-3) and the base with a transition metal compound represented by formula (8) (hereinafter, referred to as a “transition metal compound (8)”):

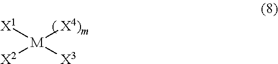

wherein M, X¹, X² and X³ are as defined above; and X⁴ is as defined in X¹, X² and X³.

Hereinafter, the step of reacting the substituted cyclopentadiene compound (7-3) with a base in the presence of an amine compound may be referred to as a “1st reaction step”, and the step of reacting the reaction product of the substituted cyclopentadiene compound (7-3) and the base with a transition metal compound (8) may be referred to as a “2nd reaction step”.

Isomers of the substituted cyclopentadiene compound (7-3) differing in the double bond position of the cyclopentadiene ring include the following structural isomers:

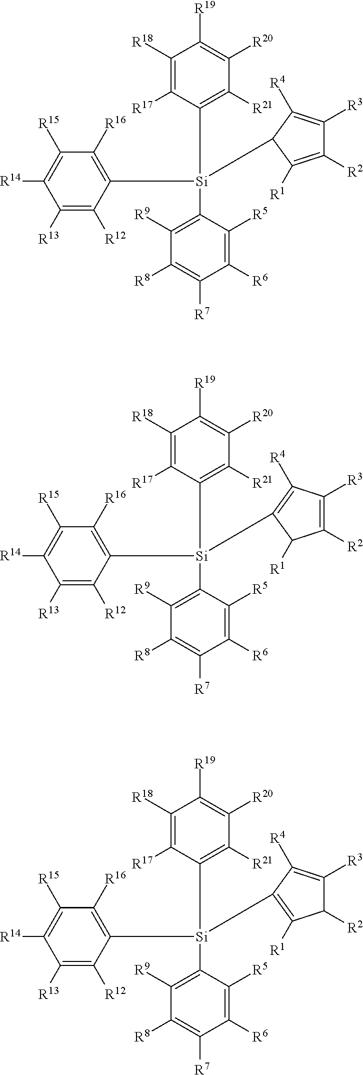

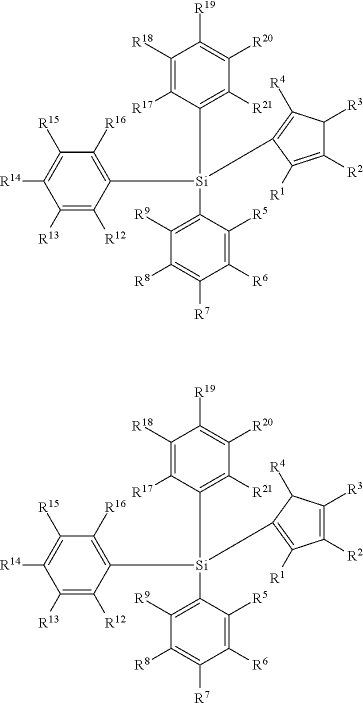

The substituted cyclopentadiene compound (7-3) has isomers differing in the double bond position of each cyclopentadiene ring. In the present invention, it represents any of them or a mixture of any of them.

In the transition metal compound (8), the substituent X⁴ is as defined above, and examples thereof can include the same as those exemplified for X¹, X² and X³.

Examples of the transition metal compound (8) include: titanium halide such as titanium tetrachloride, titanium trichloride, titanium tetrabromide and titanium tetraiodide; amidotitanium such as tetrakis(dimethylamino)titanium, dichlorobis(dimethylamino)titanium, trichloro(dimethylamino)titanium and tetrakis(diethylamino)titanium; and alkoxytitanium such as tetraisopropoxytitanium, tetra-n-butoxytitanium, dichlorodiisopropoxytitanium and trichloroisopropoxytitanium. Moreover, examples of the transition metal compound (8) include compounds obtained by substituting “zirconium” or “hafnium” for “titanium” in these compounds. Of them, a preferable transition metal compound (8) is titanium tetrachloride.

Examples of the base reacted with the substituted cyclopentadiene compound (7-3) in the 1st reaction step include organic alkali metal compounds typified by organic lithium compounds such as methyllithium, ethyllithium, n-butyllithium, sec-butyllithium, tert-butyllithium, lithiumtrimethylsilyl acetylide, lithium acetylide, trimethylsilylmethyllithium, vinyllithium, phenyllithium and allyllithium.

The amount of the base used may be in the range of 0.5 to 5 moles per mole of the substituted cyclopentadienyl compound (7-3).

In the reaction of the substituted cyclopentadiene compound (7-3) with the base in the 1st reaction step, an amine compound is used. Examples of such an amine compound include: primary amine compounds such as methylamine, ethylamine, n-propylamine, isopropylamine, n-butylamine, tert-butylamine, n-octylamine, n-decylamine, aniline and ethylenediamine; secondary amine compounds such as dimethylamine, diethylamine, di-n-propylamine, diisopropylamine, di-n-butylamine, di-tert-butylamine, di-n-octylamine, di-n-decylamine, pyrrolidine, hexamethyldisilazane and diphenylamine; and tertiary amine compounds such as trimethylamine, triethylamine, tri-n-propylamine, tri-n-butylamine, diisopropylethylamine, tri-n-octylamine, tri-n-decylamine, triphenylamine, N,N-dimethylaniline, N,N,N′,N′-tetramethylethylenediamine, N-methylpyrrolidine and 4-dimethylaminopyridine. The amount of such an amine compound used is preferably 10 moles or smaller, more preferably in the range of 0.5 to 10 moles, even more preferably in the range of 1 to 5 moles, per mole of the base.

In the 1st reaction step, the reaction of the substituted cyclopentadiene compound (7-3) with the base is preferably performed in the presence of a solvent. Moreover, when the solvent is used, the substituted cyclopentadiene compound (7-3) and the base are reacted in the solvent and then a transition metal compound (8) can be added into this reaction mixture to thereby further react the transition metal compound (8) with the reaction product of the substituted cyclopentadiene compound (7-1) and the base. Solids may be deposited in the reaction mixture obtained by reacting the substituted cyclopentadiene compound (7-3) and the base. In this case, the solvent may be further added until the deposited solid is dissolved; or the deposited solid may be temporarily separated by filtration or the like, and the solvent may be added to the separated solid for dissolution or suspension, followed by the addition of a transition metal compound (8). Moreover, when the solvent is used, the substituted cyclopentadiene compound (7-3), the base and the transition metal compound (8) can also be added simultaneously to the solvent to thereby perform the 1st reaction step and the 2nd reaction step almost simultaneously.

In the 1st reaction step, the reaction of the substituted cyclopentadiene compound (7-3) with the base is preferably performed in the presence of a solvent. Moreover, when the solvent is used, the substituted cyclopentadiene compound (7-3) and the base are reacted in the solvent and then a transition metal compound (8) can be added into this reaction mixture to thereby further react the transition metal compound (8) with the reaction product of the substituted cyclopentadiene compound (7-1) and the base. Solids may be deposited in the reaction mixture obtained by reacting the substituted cyclopentadiene compound (7-3) and the base. In this case, the solvent may be further added until the deposited solid is dissolved; or the deposited solid may be temporarily separated by filtration or the like, and the solvent may be added to the separated solid for dissolution or suspension, followed by the addition of a transition metal compound (8). Moreover, when the solvent is used, the substituted cyclopentadiene compound (7-3), the base, the amine compound and the transition metal compound (8) can also be added simultaneously to the solvent to thereby perform the 1st reaction step and the 2nd reaction step almost simultaneously.

The solvent used in the 1st reaction step or in the 1st and 2nd reaction steps is an inert solvent that does not significantly hinder the progress of the reaction associated with these steps. Examples of such a solvent include aprotic solvents such as: aromatic hydrocarbyl solvents such as benzene and toluene; aliphatic hydrocarbyl solvents such as hexane and heptane; ether solvents such as diethyl ether, tetrahydrofuran and 1,4-dioxane; amide solvents such as hexamethylphosphoric amide and dimethylformamide; polar solvents such as acetonitrile, propionitrile, acetone, diethyl ketone, methyl isobutyl ketone and cyclohexanone; and halogen solvents such as dichloromethane, dichloroethane, chlorobenzene and dichlorobenzene. These solvents can be used alone or as a mixture of two or more thereof, and the amount thereof used is preferably 1 to 200 parts by weight, more preferably 3 to 50 parts by weight, per part by weight of the substituted cyclopentadiene compound (7-3).

The amount of the transition metal compound (8) used is preferably in the range of 0.5 to 3 moles, more preferably in the range of 0.7 to 1.5 moles, per mole of the substituted cyclopentadiene compound (7-3).

The reaction temperature of the 1st and 2nd reaction steps needs only to be a temperature between −100° C. and the boiling point of the solvent inclusive and is preferably in the range of −80 to 100° C.

From the reaction mixture thus obtained through the 1st and 2nd reaction steps, the produced transition metal complex (1-3) can be taken by various purification methods known in the art. For example, the transition metal complex (1-3) of interest can be obtained by a method in which after the 1st and 2nd reaction steps, the formed precipitates are filtered off, and the filtrate is then concentrated to deposit a transition metal complex (1-3), which is then collected by filtration.

Moreover, a compound wherein any one of X¹, X² and X³ in the transition metal complex (1-3) is

an alkyl group having 1 to 20 carbon atoms which may have a halogen atom as a substituent, an alkoxy group having 1 to 20 carbon atoms which may have a halogen atom as a substituent, an aryl group having 6 to 20 carbon atoms which may have a halogen atom as a substituent, an aryloxy group having 6 to 20 carbon atoms which may have a halogen atom as a substituent,
an aralkyl group having 7 to 20 carbon atoms which may have a halogen atom as a substituent, or
an aralkyloxy group having 7 to 20 carbon atoms which may have a halogen atom as a substituent
can also be obtained by reacting a compound wherein X¹, X² or X³ in the transition metal complex (1-3) is a halogen atom, with a lithium, sodium, potassium or magnesium compound having the corresponding alkyl, alkoxy, aryl, aryloxy, aralkyl or aralkyloxy group.

### <Substituted Cyclopentadiene Compounds (7-1) to (7-6)>

In the substituted cyclopentadiene compounds (7-1) to (7-6), the substituents R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰ and R²¹ are as defined above.

Examples of the substituted cyclopentadiene compound (7-1) include the following substituted cyclopentadiene compounds:

1-dimethylphenylsilyl-2,3,4,5-tetramethylcyclopentadiene, 1-diethylphenylsilyl-2,3,4,5-tetramethylcyclopentadiene, 1-phenyldi(n-propyl)silyl-2,3,4,5-tetramethylcyclopentadiene, 1-diisopropylphenylsilyl-2,3,4,5-tetramethylcyclopentadiene, 1-di(n-butyl)phenylsilyl-2,3,4,5-tetramethylcyclopentadicne, 1-di(isobutyl)phenylsilyl-2,3,4,5-tetramethylcyclopentadiene, 1-di(sec-butyl)phenylsilyl-2,3,4,5-tetramethylcyclopentadiene, 1-di(tert-butyl)phenylsilyl-2,3,4,5-tetramethylcyclopentadiene, 1-ethylmethylphenylsilyl-2,3,4,5-tetramethylcyclopentadiene, 1-methylphenyl(n-propyl)silyl-2,3,4,5-tetramethylcyclopentadiene, 1-methylphenyl(isopropyl)silyl-2,3,4,5-tetramethylcyclopentadiene, 1-n-butylmethylphenylsilyl-2,3,4,5-tetramethylcyclopentadiene, 1-isobutylmethylphenylsilyl-2,3,4,5-tetramethylcyclopentadiene, 1-sec-butylmethylphenylsilyl-2,3,4,5-tetramethylcyclopentadiene, 1-tert-butylmethylphenylsilyl-2,3,4,5-tetramethylcyclopentadiene, 1-cyclohexylmethylphenylsilyl-2,3,4,5-tetramethylcyclopentadiene, 1-methyl(n-octadecyl)phenylsilyl-2,3,4,5-tetramethylcyclopentadiene,

1-dimethyl(3,5-dimethylphenyl)silyl-2,3,4,5-tetramethylcyclopentadiene, 1-diethyl(3,5-dimethylphenyl)silyl-2,3,4,5-tetramethylcyclopentadiene, 1-(3,5-dimethylphenyl)di(n-propyl)silyl-2,3,4,5-tetramethylcyclopentadiene, 1-diisopropyl(3,5-dimethylphenyl)silyl-2,3,4,5-tetramethylcyclopentadiene, 1-di(n-butyl)(3,5-dimethylphenyl)silyl-2,3,4,5-tetramethylcyclopentadiene, 1-di(isobutyl)(3,5-dimethylphenyl)silyl-2,3,4,5-tetramethylcyclopentadiene, 1-di(sec-butyl)(3,5-dimethylphenyl)silyl-2,3,4,5-tetramethylcyclopentadiene, 1-di(tert-butyl)(3,5-dimethylphenyl)silyl-2,3,4,5-tetramethylcyclopentadiene, 1-ethylmethyl(3,5-dimethylphenyl)silyl-2,3,4,5-tetramethylcyclopentadiene, 1-methyl(3,5-dimethylphenyl)(n-propyl)silyl-2,3,4,5-tetramethylcyclopentadiene, 1-methyl(3,5-dimethylphenyl)(isopropyl)silyl-2,3,4,5-tetramethylcyclopentadiene, 1-n-butylmethyl(3,5-dimethylphenyl)silyl-2,3,4,5-tetramethylcyclopentadiene, 1-isobutylmethyl(3,5-dimethylphenyl)silyl-2,3,4,5-tetramethylcyclopentadiene, 1-sec-butylmethyl(3,5-dimethylphenyl)silyl-2,3,4,5-tetramethylcyclopentadiene, 1-tert-butylmethyl(3,5-dimethylphenyl)silyl-2,3,4,5-tetramethylcyclopentadiene, 1-cyclohexylmethyl(3,5-dimethylphenyl)silyl-2,3,4,5-tetramethylcyclopentadiene and 1-methyl(n-octadecyl)(3,5-dimethylphenyl)silyl-2,3,4,5-tetramethylcyclopentadiene.

Moreover, examples of the substituted cyclopentadiene compound (7-1) also include substituted cyclopentadiene compounds obtained by substituting “cyclopentadiene”, “2-methylcyclopentadiene”, “3-methylcyclopentadiene”, “2,3-dimethylcyclopentadiene”, “2,4-dimethylcyclopentadiene”, “2,5-dimethylcyclopentadiene”, “2,3,5-trimethylcyclopentadiene”, “2-ethylcyclopentadiene”, “3-ethylcyclopentadiene”, “2-n-propylcyclopentadiene”, “3-n-propylcyclopentadiene”, “2-isopropylcyclopentadiene”, “3-isopropylcyclopentadiene”, “2-n-butylcyclopentadiene”, “3-n-butylcyclopentadiene”, “2-sec-butylcyclopentadiene”, “3-sec-butylcyclopentadiene”, “2-tert-butylcyclopentadiene”, “3-tert-butylcyclopentadiene”, “2-phenylcyclopentadiene”, “3-phenylcyclopentadiene”, “2-benzylcyclopentadiene”, “3-benzylcyclopentadiene”, “indene”, “2-methylindene”, “fluorene”, “tetrahydroindene”, “2-methyltetrahydroindene” or “octahydrofluorene” for “2,3,4,5-tetramethylcyclopentadiene” in the substituted cyclopentadiene compounds exemplified above.

Examples of the substituted cyclopentadiene compound (7-2) include the following substituted cyclopentadiene compounds:

1-methyldiphenylsilyl-2,3,4,5-tetramethylcyclopentadiene, 1-ethyldiphenylsilyl-2,3,4,5-tetramethylcyclopentadiene, 1-n-propyldiphenylsilyl-2,3,4,5-tetramethylcyclopentadiene, 1-isopropyldiphenylsilyl-2,3,4,5-tetramethylcyclopentadiene, 1-n-butyldiphenylsilyl-2,3,4,5-tetramethylcyclopentadiene, 1-isobutyldiphenylsilyl-2,3,4,5-tetramethylcyclopentadiene, 1-sec-butyldiphenylsilyl-2,3,4,5-tetramethylcyclopentadiene, 1-tert-butyldiphenylsilyl-2,3,4,5-tetramethylcyclopentadiene, 1-cyclohexyldiphenylsilyl-2,3,4,5-tetramethylcyclopentadiene, 1-n-octadecyldiphenylsilyl-2,3,4,5-tetramethylcyclopentadiene, 1-methylphenyl(2-methylphenyl)silyl-2,3,4,5-tetramethylcyclopentadiene, 1-methylphenyl(3-methylphenyl)silyl-2,3,4,5-tetramethylcyclopentadiene, 1-methylphenyl(4-methylphenyl)silyl-2,3,4,5-tetramethylcyclopentadiene, 1-methylphenyl(2,3-dimethylphenyl)silyl-2,3,4,5-tetramethylcyclopentadiene, 1-methylphenyl(2,4-dimethylphenyl)silyl-2,3,4,5-tetramethylcyclopentadiene, 1-methylphenyl(2,5-dimethylphenyl)silyl-2,3,4,5-tetramethylcyclopentadiene, 1-methylphenyl(2,6-dimethylphenyl)silyl-2,3,4,5-tetramethylcyclopentadiene, 1-methylphenyl(3,5-dimethylphenyl)silyl-2,3,4,5-tetramethylcyclopentadiene, 1-methylphenyl(3,4,5-trimethylphenyl)silyl-2,3,4,5-tetramethylcyclopentadiene,

1-ethylphenyl(3,5-dimethylphenyl)silyl-2,3,4,5-tetramethylcyclopentadiene, 1-n-propylphenyl(3,5-dimethylphenyl)silyl-2,3,4,5-tetramethylcyclopentadiene, 1-isopropylphenyl(3,5-dimethylphenyl)silyl-2,3,4,5-tetramethylcyclopentadiene, 1-n-butylphenyl(3,5-dimethylphenyl)silyl-2,3,4,5-tetramethylcyclopentadiene, 1-isobutylphenyl(3,5-dimethylphenyl)silyl-2,3,4,5-tetramethylcyclopentadiene, 1-sec-butylphenyl(3,5-dimethylphenyl)silyl-2,3,4,5-tetramethylcyclopentadiene, 1-tert-butylphenyl(3,5-dimethylphenyl)silyl-2,3,4,5-tetramethylcyclopentadiene, 1-cyclohexylphenyl(3,5-dimethylphenyl)silyl-2,3,4,5-tetramethylcyclopentadiene, 1-n-octadecylphenyl(3,5-dimethylphenyl)silyl-2,3,4,5-tetramethylcyclopentadiene, 1-methyl(2-methylphenyl)(3,5-dimethylphenyl)silyl-2,3,4,5-tetramethylcyclopentadiene, 1-methyl(3-methylphenyl)(3,5-dimethylphenyl)silyl-2,3,4,5-tetramethylcyclopentadiene, 1-methyl(4-methylphenyl)(3,5-dimethylphenyl)silyl-2,3,4,5-tetramethylcyclopentadiene, 1-methyl(2,3-dimethylphenyl)(3,5-dimethylphenyl)silyl-2,3,4,5-tetramethylcyclopentadiene, 1-methyl(2,4-dimethylphenyl)(3,5-dimethylphenyl)silyl-2,3,4,5-tetramethylcyclopentadiene, 1-methyl(2,5-dimethylphenyl)(3,5-dimethylphenyl)silyl-2,3,4,5-tetramethylcyclopentadiene, 1-methylphenyl(2,6-dimethylphenyl)(3,5-dimethylphenyl)silyl-2,3,4,5-tetramethylcyclopentadiene, 1-methylbis(3,5-dimethylphenyl)silyl-2,3,4,5-tetramethylcyclopentadiene and 1-methyl(3,5-dimethylphenyl)(3,4,5-trimethylphenyl)silyl-2,3,4,5-tetramethylcyclopentadiene.

Moreover, examples of the substituted cyclopentadiene compound (7-2) also include substituted cyclopentadiene compounds obtained by substituting “cyclopentadiene”, “2-methylcyclopentadiene”, “3-methylcyclopentadiene”, “2,3-dimethylcyclopentadiene”, “2,4-dimethylcyclopentadiene”, “2,5-dimethylcyclopentadiene”, “2,3,5-trimethylcyclopentadiene”, “2-ethylcyclopentadiene”, “3-ethylcyclopentadiene”, “2-n-propylcyclopentadiene”, “3-n-propylcyclopentadiene”, “2-isopropylcyclopentadiene”, “3-isopropylcyclopentadiene”, “2-n-butylcyclopentadiene”, “3-n-butylcyclopentadiene”, “2-sec-butylcyclopentadiene”, “3-sec-butylcyclopentadiene”, “2-tert-butylcyclopentadiene”, “3-tert-butylcyclopentadiene”, “2-phenylcyclopentadiene”, “3-phenylcyclopentadiene”, “2-benzylcyclopentadiene”, “3-benzylcyclopentadiene”, “indene”, “2-methylindene”, “fluorene”, “tetrahydroindene”, “2-methyltetrahydroindene” or “octahydrofluorene” for “2,3,4,5-tetramethylcyclopentadiene” in the substituted cyclopentadiene compounds exemplified above.

Examples of the substituted cyclopentadiene compound (7-3) include the following substituted cyclopentadiene compounds:

1-triphenylsilyl-2,3,4,5-tetramethylcyclopentadiene, 1-phenyldi(2-methylphenyl)silyl-2,3,4,5-tetramethylcyclopentadiene, 1-phenyldi(3-methylphenyl)silyl-2,3,4,5-tetramethylcyclopentadiene, 1-phenyldi(4-methylphenyl)silyl-2,3,4,5-tetramethylcyclopentadiene, 1-phenylbis(2,3-dimethylphenyl)silyl-2,3,4,5-tetramethylcyclopentadiene, 1-phenylbis(2,4-dimethylphenyl)silyl-2,3,4,5-tetramethylcyclopentadiene, 1-phenylbis(2,5-dimethylphenyl)silyl-2,3,4,5-tetramethylcyclopentadiene, 1-phenylbis(2,6-dimethylphenyl)silyl-2,3,4,5-tetramethylcyclopentadiene, 1-phenylbis(3,5-dimethylphenyl)silyl-2,3,4,5-tetramethylcyclopentadiene, 1-phenylbis(3,4,5-trimethylphenyl)silyl-2,3,4,5-tetramethylcyclopentadiene,

1-diphenyl(2-methylphenyl)silyl-2,3,4,5-tetramethylcyclopentadiene, 1-diphenyl(3-methylphenyl)silyl-2,3,4,5-tetramethylcyclopentadiene, 1-diphenyl(4-methylphenyl)silyl-2,3,4,5-tetramethylcyclopentadiene, 1-diphenyl(2,3-dimethylphenyl)silyl-2,3,4,5-tetramethylcyclopentadiene, 1-diphenyl(2,4-dimethylphenyl)silyl-2,3,4,5-tetramethylcyclopentadiene, 1-diphenyl(2,5-dimethylphenyl)silyl-2,3,4,5-tetramethylcyclopentadiene, 1-diphenyl(2,6-dimethylphenyl)silyl-2,3,4,5-tetramethylcyclopentadiene, 1-diphenyl(3,5-dimethylphenyl)silyl-2,3,4,5-tetramethylcyclopentadiene, 1-diphenyl(3,4,5-trimethylphenyl)silyl-2,3,4,5-tetramethylcyclopentadiene,

1-phenyl(2-methylphenyl)(3,5-dimethylphenyl)silyl-2,3,4,5-tetramethylcyclopentadiene, 1-phenyl(3-methylphenyl)(3,5-dimethylphenyl)silyl-2,3,4,5-tetramethylcyclopentadiene, 1-phenyl(4-methylphenyl)(3,5-dimethylphenyl)silyl-2,3,4,5-tetramethylcyclopentadiene, 1-phenyl(2,3-dimethylphenyl)(3,5-dimethylphenyl)silyl-2,3,4,5-tetramethylcyclopentadiene, 1-phenyl(2,4-dimethylphenyl)(3,5-dimethylphenyl)silyl-2,3,4,5-tetramethylcyclopentadiene, 1-phenyl(2,5-dimethylphenyl)(3,5-dimethylphenyl)silyl-2,3,4,5-tetramethylcyclopentadiene, 1-phenyl(2,6-dimethylphenyl)(3,5-dimethylphenyl)silyl-2,3,4,5-tetramethylcyclopentadiene, 1-phenyl(3,5-dimethylphenyl)(3,4,5-trimethylphenyl)silyl-2,3,4,5-tetramethylcyclopentadiene,

1-di(2-methylphenyl)(3,5-dimethylphenyl)silyl-2,3,4,5-tetramethylcyclopentadiene, 1-di(3-methylphenyl)(3,5-dimethylphenyl) silyl-2,3,4,5-tetramethylcyclopentadiene, 1-di(4-methylphenyl)(3,5-dimethylphenyl)silyl-2,3,4,5-tetramethylcyclopentadiene, 1-bis(2,3-dimethylphenyl)(3,5-dimethylphenyl)silyl-2,3,4,5-tetramethylcyclopentadiene, 1-bis(2,4-dimethylphenyl)(3,5-dimethylphenyl)silyl-2,3,4,5-tetramethylcyclopentadiene, 1-bis(2,5-dimethylphenyl)(3,5-dimethylphenyl)silyl-2,3,4,5-tetramethylcyclopentadiene, 1-bis(2,6-dimethylphenyl)(3,5-dimethylphenyl)silyl-2,3,4,5-tetramethylcyclopentadiene, 1-tris(3,5-dimethylphenyl)silyl-2,3,4,5-tetramethylcyclopentadiene and 1-(3,5-dimethylphenyl)bis(3,4,5-dimethylphenyl)silyl-2,3,4,5-tetramethylcyclopentadiene.

Moreover, examples of the substituted cyclopentadiene compound (7-3) also include substituted cyclopentadiene compounds obtained by substituting “cyclopentadiene”, “2-methylcyclopentadiene”, “3-methylcyclopentadiene”, “2,3-dimethylcyclopentadiene”, “2,4-dimethylcyclopentadiene”, “2,5-dimethylcyclopentadiene”, “2,3,5-trimethylcyclopentadiene”, “2-ethylcyclopentadiene”, “3-ethylcyclopentadiene”, “2-n-propylcyclopentadiene”, “3-n-propylcyclopentadiene”, “2-isopropylcyclopentadiene”, “3-isopropylcyclopentadiene”, “2-n-butylcyclopentadiene”, “3-n-butylcyclopentadiene”, “2-sec-butylcyclopentadiene”, “3-sec-butylcyclopentadiene”, “2-tert-butylcyclopentadiene”, “3-tert-butylcyclopentadiene”, “2-phenylcyclopentadiene”, “3-phenylcyclopentadiene”, “2-benzylcyclopentadiene”, “3-benzylcyclopentadiene”, “indene”, “2-methylindene”, “fluorene”, “tetrahydroindene”, “2-methyltetrahydroindene” or “octahydrofluorene” for “2,3,4,5-tetramethylcyclopentadiene” in the substituted cyclopentadiene compounds exemplified above.

Examples of the substituted cyclopentadiene compound (7-4) include the following substituted cyclopentadiene compounds:

1-phenylmethyl-cyclopentadiene, 1-(2-methylphenyl)methyl-cyclopentadiene, 1-(3-methylphenyl)methyl-cyclopentadiene, 1-(4-methylphenyl)methyl-cyclopentadiene, dimethylphenyl)methyl-cyclopentadiene, 1-(2,4-dimethylphenyl)methyl-cyclopentadiene, 1-(2,5-dimethylphenyl)methyl-cyclopentadiene, 1-(2,6-dimethylphenyl)methyl-cyclopentadiene, 1-(3,4-dimethylphenyl)methyl-cyclopentadiene, 1-(3,5-dimethylphenyl)methyl-cyclopentadiene, 1-(2,3,4-trimethylphenyl)methyl-cyclopentadiene, [1-(2,3,5-trimethylphenyl)methyl-cyclopentadiene, 1-(2,3,6-trimethylphenyl)methyl-cyclopentadiene, 1-(2,4,5-trimethylphenyl)methyl-cyclopentadiene, 1-(2,4,6-trimethylphenyl)methyl-cyclopentadiene, 1-(3,4,5-trimethylphenyl)methyl-cyclopentadiene, 1-(2,3,4,5,6-pentamethylphenyl)methyl-cyclopentadiene, 1-(2-trimethylsilylphenyl)methyl-cyclopentadiene, 1-(3-trimethylsilylphenyl)methyl-cyclopentadiene, 1-(4-trimethylsilylphenyl)methyl-cyclopentadiene, 1-(1-methyl-1-phenylethyl)-cyclopentadiene, 1-(1-methyl-1-(2-methylphenyl)ethyl)-cyclopentadiene, 1-(1-methyl-1-(3-methylphenyl)ethyl)-cyclopentadiene, 1-(1-methyl-1-(4-methylphenyl)ethyl)-cyclopentadiene, 1-(1-methyl-1-(2,3-dimethylphenyl)ethyl)-cyclopentadiene, 1-(1-methyl-1-(2,4-dimethylphenyl)ethyl)-cyclopentadiene, 1-(1-methyl-1-(2,5-dimethylphenyl)ethyl)-cyclopentadiene, 1-(1-methyl-1-(2,6-dimethylphenyl)ethyl)-cyclopentadiene, 1-(1-methyl-1-(3,4-dimethylphenyl)ethyl)-cyclopentadiene, 1-(1-methyl-1-(3,5-dimethylphenyl)ethyl)-cyclopentadiene, 1-(1-methyl-1-(2,3,4-trimethylphenyl)ethyl)-cyclopentadiene, 1-(1-methyl-1-(2,3,5-trimethylphenyl)ethyl)-cyclopentadiene, 1-(1-methyl-1-(2,3,6-trimethylphenyl)ethyl)-cyclopentadiene, 1-(1-methyl-1-(2,4,5-trimethylphenyl)ethyl)-cyclopentadiene, 1-(1-methyl-1-(2,4,6-trimethylphenyl)ethyl)-cyclopentadiene, 1-(1-methyl-1-(3,4,5-trimethylphenyl)ethyl)-cyclopentadiene, 1-(1-methyl-1-(2,3,4,5,6-pentamethylphenyl)ethyl)-cyclopentadiene, 1-(1-ethyl-1-phenylpropyl)-cyclopentadiene, 141-phenylcyclohexyl)-cyclopentadiene, 1-(1-phenylvinyl)-cyclopentadiene,

1-phenylmethyl-3-trimethylsilylcyclopentadiene, 1-(2-methylphenyl)methyl-3-trimethylsilylcyclopentadiene, 1-(3-methylphenyl)methyl-3-trimethylsilylcyclopentadiene, 1-(4-methylphenyl)methyl-3-trimethylsilylcyclopentadiene, 1-(2,3-dimethylphenyl)methyl-3-trimethylsilylcyclopentadiene, 1-(2,4-dimethylphenyl)methyl-3-trimethylsilylcyclopentadiene, 1-(2,5-dim ethylphenyl)methyl-3-trimethylsilylcyclopentadiene, 1-(2,6-dimethylphenyl)methyl-3-trimethylsilylcyclopentadiene, 1-(3,4-dimethylphenyl)methyl-3-trimethylsilylcyclopentadiene, 1-(3,5-dimethylphenyl)methyl-3-trimethylsilylcyclopentadiene, 1-(2,3,4-trimethylphenyl)methyl-3-trimethylsilylcyclopentadiene, 1-(2,3,5-trimethylphenyl)methyl-3-trimethylsilylcyclopentadiene, 1-(2,3,6-trimethylphenyl)methyl-3-trimethylsilylcyclopentadiene, 1-(2,4,5-trimethylphenyl)methyl-3-trimethylsilylcyclopentadiene, 1-(2,4,6-trimethylphenyl)methyl-3-trimethylsilylcyclopentadiene, 1-(3,4,5-trimethylphenyl)methyl-3-trimethylsilylcyclopentadiene, 1-(2,3,4,5,6-pentamethylphenyl)methyl-3-trimethylsilylcyclopentadiene, 1-(2-trimethylsilylphenyl)methyl-3-trimethylsilylcyclopentadiene, 1-(3-trimethylsilylphenyl)methyl-3-trimethylsilylcyclopentadiene, 1-(4-trimethylsilylphenyl)methyl-3-trimethylsilylcyclopentadiene, 1-(1-methyl-1-phenylethyl)-3-trimethylsilylcyclopentadiene, 1-(1-methyl-1-(2-methylphenyl)ethyl)-3-trimethylsilylcyclopentadiene, 1-(1-methyl-1-(3-methylphenyl)ethyl)-3-trimethylsilylcyclopentadiene, 1-(1-methyl-1-(4-methylphenyl)ethyl)-3-trimethylsilylcyclopentadiene, 1-(1-methyl-1-(2,3-dimethylphenyl)ethyl)-3-trimethylsilylcyclopentadiene, 1-(1-methyl-1-(2,4-dimethylphenyl)ethyl)-3-trimethylsilylcyclopentadiene, 1-(1-methyl-1-(2,5-dimethylphenyl)ethyl)-3-trimethylsilylcyclopentadiene, 1-(1-methyl-1-(2,6-dimethylphenyl)ethyl)-3-trimethylsilylcyclopentadiene, 1-(1-methyl-1-(3,4-dimethylphenyl)ethyl)-3-trimethylsilylcyclopentadiene, 1-(1-methyl-1-(3,5-dimethylphenyl)ethyl)-3-trimethylsilylcyclopentadiene, 1-(1-methyl-1-(2,3,4-trimethylphenyl)ethyl)-3-trimethylsilylcyclopentadiene, 1-(1-methyl-1-(2,3,5-trimethylphenyl)ethyl)-3-trimethylsilylcyclopentadiene, 1-(1-methyl-1-(2,3,6-trimethylphenyl)ethyl)-3-trimethylsilylcyclopentadiene, 1-(1-methyl-1-(2,4,5-trimethylphenyl)ethyl)-3-trimethylsilylcyclopentadiene, 1-(1-methyl-1-(2,4,6-trimethylphenyl)ethyl)-3-trimethylsilylcyclopentadiene, 1-(1-methyl-1-(3,4,5-trimethylphenyl)ethyl)-3-trimethylsilylcyclopentadiene, 1-(1-methyl-1-(2,3,4,5,6-pentamethylphenyl)ethyl)-3-trimethylsilylcyclopentadiene, 1-(1-ethyl-1-phenylpropyl)-3-trimethylsilylcyclopentadiene and 1-(1-phenylcyclohexyl)-3-trimethylsilylcyclopentadiene and 1-(1-phenylvinyl)-3-trimethylsilylcyclopentadiene.

Examples of the substituted cyclopentadiene compound (7-5) include the following substituted cyclopentadiene compounds:

1-diphenylmethyl-2-methyl-cyclopentadiene, 1 diphenylmethyl-3-methyl-cyclopentadiene, 1-diphenylmethyl-2,3-dimethyl-cyclopentadiene, 1-diphenylmethyl-2,4-dimethyl-cyclopentadiene, 1-diphenylmethyl-3,4-dimethyl-cyclopentadiene, 1-diphenylmethyl-2,3,4-trimethyl-cyclopentadiene, 1-diphenylmethyl-2,3,5-trimethyl-cyclopentadiene, 1-diphenylmethyl-2,3,4,5-tetramethyl-cyclopentadiene, 1-diphenylmethyl-2-ethyl-cyclopentadiene, 1-diphenylmethyl-3-ethyl-cyclopentadiene, 1-diphenylmethyl-2-n-propyl-cyclopentadiene, 1-diphenylmethyl-3-n-propyl-cyclopentadiene, 1-diphenylmethyl-2-isopropyl-cyclopentadiene, 1-diphenylmethyl-3-isopropyl-cyclopentadiene, 1-diphenylmethyl-2-n-butyl-cyclopentadiene, 1-diphenylmethyl-3-n-butyl-cyclopentadiene, 1-diphenylmethyl-2-sec-butyl-cyclopentadiene, 1-diphenylmethyl-3-sec-butyl-cyclopentadiene, 1-diphenylmethyl-2-tert-butyl-cyclopentadiene, 1-diphenylmethyl-3-tert-butyl-cyclopentadiene, 1-diphenylmethyl-2-phenyl-cyclopentadiene, 1-diphenylmethyl-3-phenyl-cyclopentadiene, 1-diphenylmethyl-2-benzyl-cyclopentadiene, 1-diphenylmethyl-3-benzyl-cyclopentadiene, 1-diphenylmethyl-indene, 1-diphenylmethyl-2-methylindene, 1-diphenylmethyl-tetrahydroindene, 9-diphenylmethyl-fluorene, 1-diphenylmethyl-2-methyltetrahydroindene, 9-diphenylmethyl-octahydrofluorene,

1-di(3-methylphenyl)methyl-cyclopentadiene, 1-di(3-methylphenyl)methyl-2-methyl-cyclopentadiene, 1-di(3-methylphenyl)methyl-3-methyl-cyclopentadiene, 1-di(3-methylphenyl)methyl-2,3-dimethyl-cyclopentadiene, 1-di(3-methylphenyl)methyl-2,4-dimethyl-cyclopentadiene, 1-di(3-methylphenyl)methyl-3,4-dimethyl-cyclopentadiene, 1-di(3-methylphenyl)methyl-2,3,4-trimethyl-cyclopentadiene, 1-di(3-methylphenyl)methyl-2,3,5-trimethyl-cyclopentadiene, 1-di(3-methylphenyl)methyl-2,3,4,5-tetramethyl-cyclopentadiene, 1-di(3-methylphenyl)methyl-2-ethyl-cyclopentadiene, 1-di(3-methylphenyl)methyl-3-ethyl-cyclopentadiene, 1-di(3-methylphenyl)methyl-2-n-propyl-cyclopentadiene, 1-di(3-methylphenyl)methyl-3-n-propyl-cyclopentadiene, 1-di(3-methylphenyl)methyl-2-isopropyl-cyclopentadiene, 1-di(3-methylphenyl)methyl-3-isopropyl-cyclopentadiene, 1-di(3-methylphenyl)methyl-2-n-butyl-cyclopentadiene, 1-di(3-methylphenyl)methyl-3-n-butyl-cyclopentadiene, 1-di(3-methylphenyl)methyl-2-sec-butyl-cyclopentadiene, 1-di(3-methylphenyl)methyl-3-sec-butyl-cyclopentadiene, 1-di(3-methylphenyl)methyl-2-tert-butyl-cyclopentadiene, 1-di(3-methylphenyl)methyl-3-tert-butyl-cyclopentadiene, 1-di(3-methylphenyl)methyl-2-phenyl-cyclopentadiene, 1-di(3-methylphenyl)methyl-3-phenyl-cyclopentadiene, 1-di(3-methylphenyl)methyl-2-benzyl-cyclopentadiene, 1-di(3-methylphenyl)methyl-3-benzyl-cyclopentadiene, 1-di(3-methylphenyl)methyl-indene, 1-di(3-methylphenyl)methyl-2-methylindene, 9-di(3-methylphenyl)methyl-fluorene, 1-di(3-methylphenyl)methyl-tetrahydroindene, 1-di(3-methylphenyl)methyl-2-methyltetrahydroindene, 9-di(3-methylphenyl)methyl-octahydrofluorene,

1-bis(3,5-dimethylphenyl)methyl-cyclopentadiene, 1-bis(3,5-dimethylphenyl)methyl-2-methyl-cyclopentadiene, 1-bis(3,5-dimethylphenyl)methyl-3-methyl-cyclopentadiene, 1-bis(3,5-dimethylphenyl)methyl-2,3-dimethyl-cyclopentadiene, 1-bis(3,5-dimethylphenyl)methyl-2,4-dimethyl-cyclopentadiene, 1-bis(3,5-dimethylphenyl)methyl-3,4-dimethyl-cyclopentadiene, 1-bis(3,5-dimethylphenyl)methyl-2,3,4-trimethyl-cyclopentadiene, 1-bis(3,5-dimethylphenyl)methyl-2,3,5-trimethyl-cyclopentadiene, 1-bis(3,5-dimethylphenyl)methyl-2,3,4,5-tetramethyl-cyclopentadiene, 1-bis(3,5-dimethylphenyl)methyl-2-ethyl-cyclopentadiene, 1-bis(3,5-dimethylphenyl)methyl-3-ethyl-cyclopentadiene, 1-bis(3,5-dimethylphenyl)methyl-2-n-propyl-cyclopentadiene, 1-bis(3,5-dimethylphenyl)methyl-3-n-propyl-cyclopentadiene, 1-bis(3,5-dimethylphenyl)methyl-2-isopropyl-cyclopentadiene, 1-bis(3,5-dimethylphenyl)methyl-3-isopropyl-cyclopentadiene, 1-bis(3,5-dimethylphenyl)methyl-2-n-butyl-cyclopentadiene, 1-bis(3,5-dimethylphenyl)methyl-3-n-butyl-cyclopentadiene, 1-bis(3,5-dimethylphenyl)methyl-2-sec-butyl-cyclopentadiene, 1-bis(3,5-dimethylphenyl)methyl-3-sec-butyl-cyclopentadiene, 1-bis(3,5-dimethylphenyl)methyl-2-tert-butyl-cyclopentadiene, 1-bis(3,5-dimethylphenyl)methyl-3-tert-butyl-cyclopentadiene, 1-bis(3,5-dimethylphenyl)methyl-2-phenyl-cyclopentadiene, 1-bis(3,5-dimethylphenyl)methyl-3-phenyl-cyclopentadiene, 1-bis(3,5-dimethylphenyl)methyl-2-benzyl-cyclopentadiene, 1-bis(3,5-dimethylphenyl)methyl-3-benzyl-cyclopentadiene, 1-bis(3,5-dimethylphenyl)methyl-indene, 1-bis(3,5-dimethylphenyl)methyl-2-methylindene, 9-bis(3,5-dimethylphenyl)methyl-fluorene, 1-bis(3,5-dimethylphenyl)methyl-tetrahydroindene, 1-bis(3,5-dimethylphenyl)methyl-2-methyltetrahydroindene, 9-bis(3,5-dimethylphenyl)methyl-octahydrofluorene,

1-(1,1-diphenyl-ethyl)-cyclopentadiene, 1-(1,1-diphenyl-ethyl)-2-methyl-cyclopentadiene, 1-(1,1-diphenyl-ethyl)-3-methyl-cyclopentadiene, 1-(1,1-diphenyl-ethyl)-2,3-dimethyl-cyclopentadiene, 1-(1,1-diphenyl-ethyl)-2,4-dimethyl-cyclopentadiene, 1-(1,1-diphenyl-ethyl)-3,4-dimethyl-cyclopentadiene, 1-(1,1-diphenyl-ethyl)-2,3,4-trimethyl-cyclopentadiene, 1-(1,1-diphenyl-ethyl)-2,3,5-trimethyl-cyclopentadiene, 1-(1,1-diphenyl-ethyl)-2,3,4,5-tetramethyl-cyclopentadiene, 1-(1,1-diphenyl-ethyl)-2-ethyl-cyclopentadiene, 1-(1,1-diphenyl-ethyl)-3-ethyl-cyclopentadiene, 1-(1,1-diphenylethyl)-2-n-propyl-cyclopentadiene, 1-(1,1-diphenyl-ethyl)-3-n-propyl-cyclopentadiene, 1-(1,1-diphenyl-ethyl)-2-isopropyl-cyclopentadiene, 1-(1,1-diphenyl-ethyl)-3-isopropyl-cyclopentadiene, 1-(1,1-diphenyl-ethyl)-2-n-butyl-cyclopentadiene, 1-(1,1-diphenylethyl)-3-n-butyl-cyclopentadiene, 1-(1,1-diphenyl-ethyl)-2-sec-butyl-cyclopentadiene, 1-(1,1-diphenyl-ethyl)-3-sec-butyl-cyclopentadiene, diphenyl-ethyl)-2-tert-butyl-cyclopentadiene, 1-(1,1-diphenyl-ethyl)-3-tert-butyl-cyclopentadiene, 1-(1,1-diphenyl-ethyl)-2-phenyl-cyclopentadiene, 1-(1,1-diphenyl-ethyl)-3-phenyl-cyclopentadiene, 1-(1,1-diphenyl-ethyl)-2-benzyl-cyclopentadiene, 1-(1,1-diphenyl-ethyl)-3-benzyl-cyclopentadiene, 1-(1,1-diphenyl-ethyl)-indene, 1-(1,1-diphenyl-ethyl)-2-methylindene, 1-(1,1-diphenyl-ethyl)-tetrahydroindene, 9-(1,1-diphenyl-ethyl)-fluorene, 1-(1,1-diphenyl-ethyl)-2-methyltetrahydroindene, 9-(1,1-diphenyl-ethyl)-octahydrofluorene,

1-(1,1-diphenyl-pentyl)-cyclopentadiene, 1-(1,1-diphenyl-pentyl)-2-methyl-cyclopentadiene, 1-(1,1-diphenyl-pentyl)-3-methyl-cyclopentadiene, 1-(1,1-diphenyl-pentyl)-2,3-dimethyl-cyclopentadiene, 1-(1,1-diphenyl-pentyl)-2,4-dimethyl-cyclopentadiene, 1-(1,1-diphenyl-pentyl)-3,4-dimethyl-cyclopentadiene, 1-(1,1-diphenyl-pentyl)-2,3,4-trimethyl-cyclopentadiene, 1-(1,1-diphenyl-pentyl)-2,3,5-trimethyl-cyclopentadiene, 1-(1,1-diphenyl-pentyl)-2,3,4,5-tetramethyl-cyclopentadiene, 1-(1,1-diphenyl-pentyl)-2-ethyl-cyclopentadiene, 1-(1,1-diphenyl-pentyl)-3-ethyl-cyclopentadiene, 1-(1,1-diphenyl-pentyl)-2-n-propyl-cyclopentadiene, 1-(1,1-diphenyl-pentyl)-3-n-propyl-cyclopentadiene, 1-(1,1-diphenyl-pentyl)-2-isopropyl-cyclopentadiene, 1-(1,1-diphenyl-pentyl)-3-isopropyl-cyclopentadiene, 1-(1,1-diphenyl-pentyl)-2-n-butyl-cyclopentadiene, 1-(1,1-diphenyl-pentyl)-3-n-butyl-cyclopentadiene, 1-(1,1-diphenyl-pentyl)-2-sec-butyl-cyclopentadiene, 1-(1,1-diphenyl-pentyl)-3-sec-butyl-cyclopentadiene, 1-(1,1-diphenyl-pentyl)-2-tert-butyl-cyclopentadiene, 1-(1,1-diphenyl-pentyl)-3-tert-butyl-cyclopentadiene, 1-(1,1-diphenyl-pentyl)-2-phenyl-cyclopentadiene, 1-(1,1-diphenyl-pentyl)-3-phenyl-cyclopentadiene, 1-(1,1-diphenyl-pentyl)-2-benzyl-cyclopentadiene, 1-(1,1-diphenyl-pentyl)-3-benzyl-cyclopentadiene, 1-(1,1-diphenyl-pentyl)-indene, 1-(1,1-diphenyl-pentyl)-2-methylindene, 1-(1,1-diphenyl-pentyl)-tetrahydroindene, 9-(1,1-diphenyl-pentyl)-fluorene, 1-(1,1-diphenyl-pentyl)-2-methyltetrahydroindene, [9-(1,1-diphenyl-pentyl)-octahydrofluorene,

1-(1,1-diphenyl-1-tert-butyl-methyl)-cyclopentadiene, 1-(1,1-diphenyl-1-tert-butyl-methyl)-2-methyl-cyclopentadiene, 1-(1,1-diphenyl-1-tert-butyl-methyl)-3-methyl-cyclopentadiene, 1-(1,1-diphenyl-1-tert-butyl-methyl)-2,3-dimethyl-cyclopentadiene, 1-(1,1-diphenyl-1-tert-butyl-methyl)-2,4-dimethyl-cyclopentadiene, 1-(1,1-diphenyl-1-tert-butyl-methyl)-3,4-dimethyl-cyclopentadiene, 1-(1,1-diphenyl-1-tert-butyl-methyl)-2,3,4-trimethyl-cyclopentadiene, 1-(1,1-diphenyl-1-tert-butyl-methyl)-2,3,5-trimethyl-cyclopentadiene, 1-(1,1-diphenyl-1-tert-butyl-methyl)-2,3,4,5-tetramethyl-cyclopentadiene, 1-(1,1-diphenyl-1-tert-butyl-methyl)-2-ethyl-cyclopentadiene, 1-(1,1-diphenyl-1-tert-butyl-methyl)-3-ethyl-cyclopentadiene, 1-(1,1-diphenyl-1-tert-butyl-methyl)-2-n-propyl-cyclopentadiene, 1-(1,1-diphenyl-1-tert-butyl-methyl)-3-n-propyl-cyclopentadiene, 1-(1,1-diphenyl-1-tert-butyl-methyl)-2-isopropyl-cyclopentadiene, 1-(1,1-diphenyl-1-tert-butyl-methyl)-3-isopropyl-cyclopentadiene, 1-(1,1-diphenyl-1-tert-butyl-methyl)-2-n-butyl-cyclopentadiene, 1-(1,1-diphenyl-1-tert-butyl-methyl)-3-n-butyl-cyclopentadiene, 1-(1,1-diphenyl-1-tert-butyl-methyl)-2-sec-butyl-cyclopentadiene, 1-(1,1-diphenyl-1-tert-butyl-methyl)-3-sec-butyl-cyclopentadiene, 1-(1,1-diphenyl-1-tert-butyl-methyl)-2-tert-butyl-cyclopentadiene, 1-(1,1-diphenyl-1-tert-butyl-methyl)-3-tert-butyl-cyclopentadiene, 1-(1,1-diphenyl-1-tert-butyl-methyl)-2-phenyl-cyclopentadiene, 1-(1,1-diphenyl-1-tert-butyl-methyl)-3-phenyl-cyclopentadiene, 1-(1,1-diphenyl-1-tert-butyl-methyl)-2-benzyl-cyclopentadiene, 1-(1,1-diphenyl-1-tert-butyl-methyl)-3-benzyl-cyclopentadiene, 1-(1,1-diphenyl-1-tert-butyl-methyl)-indene, 1-(1,1-diphenyl-1-tert-butyl-methyl)-2-methylindene, 1-(1,1-diphenyl-1-tert-butyl-methyl)-tetrahydroindene, 9-(1,1-diphenyl-1-tert-butyl-methyl)-fluorene, 1-(1,1-diphenyl-1-tert-butyl-methyl)-2-methyltetrahydroindene and 9-(1,1-diphenyl-1-1.5 tert-butyl-methyl)-octahydrofluorene.

Examples of the substituted cyclopentadiene compound (7-6) include the following substituted cyclopentadiene compounds:

1-triphenylmethyl-2-methyl-cyclopentadiene, 1-triphenylmethyl-3-methyl-cyclopentadiene, 1-triphenylmethyl-2,3-dimethyl-cyclopentadiene, 1-triphenylmethyl-2,4-dimethyl-cyclopentadiene, 1-triphenylmethyl-3,4-dimethyl-cyclopentadiene, 1-triphenylmethyl-2,3,4-trimethyl-cyclopentadiene, 1-triphenylmethyl-2,3,5-trimethyl-cyclopentadiene, 1-triphenylmethyl-2,3,4,5-tetramethyl-cyclopentadiene, 1-triphenylmethyl-2-ethyl-cyclopentadiene, 1-triphenylmethyl-3-ethyl-cyclopentadiene, 1-triphenylmethyl-2-n-propyl-cyclopentadiene, 1-triphenylmethyl-3-n-propyl-cyclopentadiene, 1-triphenylmethyl-2-isopropyl-cyclopentadiene, 1-triphenylmethyl-3-isopropyl-cyclopentadiene, 1-triphenylmethyl-2-n-butyl-cyclopentadiene, 1-triphenylmethyl-3-n-butyl-cyclopentadiene, 1-triphenylmethyl-2-sec-butyl-cyclopentadiene, 1-triphenylmethyl-3-sec-butyl-cyclopentadiene, 1-triphenylmethyl-2-tert-butyl-cyclopentadiene, 1-triphenylmethyl-3-tert-butyl-cyclopentadiene, 1-triphenylmethyl-2-phenyl-cyclopentadiene, 1-triphenylmethyl-3-phenyl-cyclopentadiene, 1-triphenylmethyl-2-benzyl-cyclopentadiene, 1-triphenylmethyl-3-benzyl-cyclopentadiene, 1-triphenylmethyl-indene, 1-triphenylmethyl-2-methylindene, 9-triphenylmethyl-fluorene, 1-triphenylmethyl-tetrahydroindene, 1-triphenylmethyl-2-methyl tetrahydroindene, 9-triphenyl methyl-octahydrofluorene,

1-tri(3-methylphenyl)methyl-cyclopentadiene, 1-tri(3-methylphenyl)methyl-2-methyl-cyclopentadiene, 1-tri(3-methylphenyl)methyl-3-methyl-cyclopentadiene, 1-tri(3-methylphenyl)methyl-2,3-dimethyl-cyclopentadiene, 1-tri(3-methylphenyl)methyl-2,4-dimethyl-cyclopentadiene, 1-tri(3-methylphenyl)methyl-3,4-dimethyl-cyclopentadiene, 1-tri(3-methylphenyl)methyl-2,3,4-trimethyl-cyclopentadiene, 1-tri(3-methylphenyl)methyl-2,3,5-trimethyl-cyclopentadiene, 1-tri(3-methylphenyl)methyl-2,3,4,5-tetramethyl-cyclopentadiene, 1-tri(3-methylphenyl)methyl-2-ethyl-cyclopentadiene, 1-tri(3-methylphenyl)methyl-3-ethyl-cyclopentadiene, 1-tri(3-methylphenyl)methyl-2-n-propyl-cyclopentadiene, 1-tri(3-methylphenyl)methyl-3-n-propyl-cyclopentadiene, 1-tri(3-methylphenyl)methyl-2-isopropyl-cyclopentadiene, 1-tri(3-methylphenyl)methyl-3-isopropyl-cyclopentadiene, 1-tri(3-methylphenyl)methyl-2-n-butyl-cyclopentadiene, 1-tri(3-methylphenyl)methyl-3-n-butyl-cyclopentadiene, 1-tri(3-methylphenyl)methyl-2-sec-butyl-cyclopentadiene, 1-tri(3-methylphenyl)methyl-3-sec-butyl-cyclopentadiene, 1-tri(3-methylphenyl)methyl-2-tert-butyl-cyclopentadiene, 1-tri(3-methylphenyl)methyl-3-tert-butyl-cyclopentadiene, 1-tri(3-methylphenyl)methyl-2-phenyl-cyclopentadiene, 1-tri(3-methylphenyl)methyl-3-phenyl-cyclopentadiene, 1-tri(3-methylphenyl)methyl-2-benzyl-cyclopentadiene, 1-tri(3-methylphenyl)methyl-3-benzyl-cyclopentadiene, 1-tri(3-methylphenyl)methyl-indene, 1-tri(3-methylphenyl)methyl-2-methylindene, 9-tri(3-methylphenyl)methyl-fluorene, 1-tri(3-methylphenyl)methyl-tetrahydroindene, 1-tri(3-methylphenyl)methyl-2-methyltetrahydroindene, 9-tri(3-methylphenyl)methyl-octahydrofluorene,

1-tris(3,5-dimethylphenyl)methyl-cyclopentadiene, 1-tris(3,5-dimethylphenyl)methyl-2-methyl-cyclopentadiene, 1-tris(3,5-dimethylphenyl)methyl-3-methyl-cyclopentadiene, 1-tris(3,5-dimethylphenyl)methyl-2,3-dimethyl-cyclopentadiene, 1-tris(3,5-dimethylphenyl)methyl-2,4-dimethyl-cyclopentadiene, 1-tris(3,5-dimethylphenyl)methyl-3,4-dimethyl-cyclopentadiene, 1-tris(3,5-dimethylphenyl)methyl-2,3,4-trimethyl-cyclopentadiene, 1-tris(3,5-dimethylphenyl)methyl-2,3,5-trimethyl-cyclopentadiene, 1-tris(3,5-dimethylphenyl)methyl-2,3,4,5-tetramethyl-cyclopentadiene, 1-tris(3,5-dimethylphenyl)methyl-2-ethyl-cyclopentadiene, 1-tris(3,5-dimethylphenyl)methyl-3-ethyl-cyclopentadiene, 1-tris(3,5-dimethylphenyl)methyl-2-n-propyl-cyclopentadiene, 1-tris(3,5-dimethylphenyl)methyl-3-n-propyl-cyclopentadiene, 1-tris(3,5-dimethylphenyl)methyl-2-isopropyl-cyclopentadiene, 1-tris(3,5-dimethylphenyl)methyl-3-isopropyl-cyclopentadiene, 1-tris(3,5-dimethylphenyl)methyl-2-n-butyl-cyclopentadiene, 1-tris(3,5-dimethylphenyl)methyl-3-n-butyl-cyclopentadiene, 1-tris(3,5-dimethylphenyl)methyl-2-sec-butyl-cyclopentadiene, 1-tris(3,5-dimethylphenyl)methyl-3-sec-butyl-cyclopentadiene, 1-tris(3,5-dimethylphenyl)methyl-2-tert-butyl-cyclopentadiene, 1-tris(3,5-dimethylphenyl)methyl-3-tert-butyl-cyclopentadiene, 1-tris(3,5-dimethyl-phenyl)methyl-2-phenyl-cyclopentadiene, 1-tris(3,5-dimethylphenyl)methyl-3-phenyl-cyclopentadiene, 1-tris(3,5-dimethylphenyl)methyl-2-benzyl-cyclopentadiene, 1-tris(3,5-dimethylphenyl)methyl-3-benzyl-cyclopentadiene, 1-tris(3,5-dimethylphenyl)methyl-indene, 1-tris(3,5-dimethylphenyl)methyl-2-methylindene, 1-tris(3,5-dimethylphenyl)methyl-tetrahydroindene, 9-tris(3,5-dimethylphenyl)methyl-fluorene, 1-tris(3,5-dimethylphenyl)methyl-2-methyltetrahydroindene and 9-tris(3,5-dimethylphenyl)methyl-octahydrofluorene.

### <Methods for Producing Substituted Cyclopentadiene Compounds (7-1) to (7-6)>

The methods for producing the substituted cyclopentadiene compounds (7-1) to (7-6) will be described below by taking a substituted cyclopentadiene compound wherein J is silicon as an example.

The substituted cyclopentadiene compounds (7-1), (7-2) and (7-3) can be produced by similar methods comprising the steps of:

reacting a substituted cyclopentadiene compound represented by formula (9) (hereinafter, abbreviated to a “substituted cyclopentadiene compound (9)”) with a base; and
reacting the reaction product of the substituted cyclopentadiene compound (9) and the base with a halogenated silyl compound represented by formula (10-1) (hereinafter, abbreviated to a “halogenated silyl compound (10-1)”),
reacting the substituted cyclopentadiene compound (9) with a base; and
reacting the reaction product of the substituted cyclopentadiene compound (9) and the base with a halogenated silyl compound represented by formula (10-2) (hereinafter, abbreviated to a “halogenated silyl compound (10-2)”), and
reacting the substituted cyclopentadiene compound (9) with a base; and
reacting the reaction product of the substituted cyclopentadiene compound (9) and the base with a halogenated silyl compound represented by formula (10-3) (hereinafter, abbreviated to a “halogenated silyl compound (10-3)”), respectively:

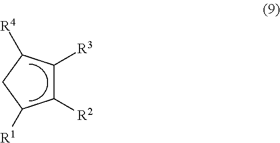

wherein R¹, R², R³ and R⁴ are as defined above, and

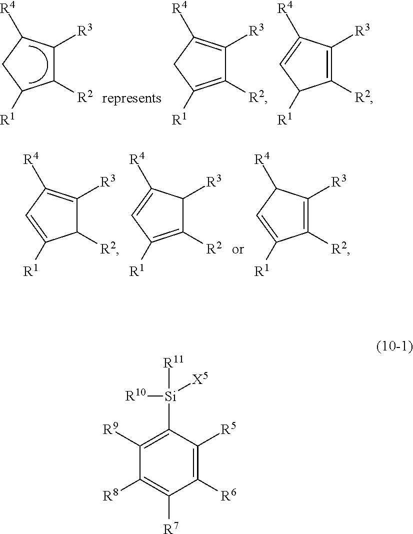

wherein R⁵, R⁶, R⁷, R⁸, R⁹, R¹⁰ and R¹¹ are as defined above, and X⁵ is a halogen atom,

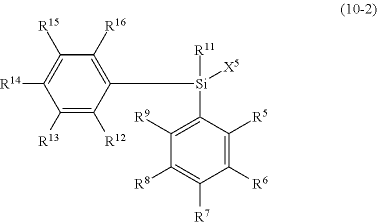

wherein R⁵, R⁶, R⁷, R⁸, R⁹, R¹¹, R¹², R¹³, R¹⁴, R¹⁵ and R¹⁶ are as defined above, and X⁵ is a halogen atom, and

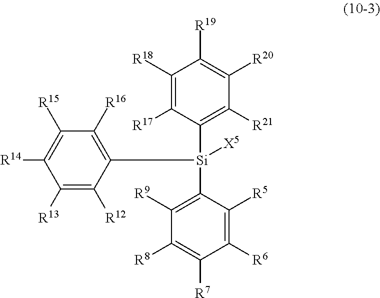

wherein R⁵, R⁶, R⁷, R⁸, R⁹, R¹², R¹³, R¹⁴, R¹⁵, R¹⁶, R¹⁷, R¹⁸, R¹⁹, R²⁰ and R²¹ are as defined above, and X⁵ is a halogen atom.

Hereinafter, a method for producing the substituted cyclopentadiene compound (7-1) will be described as an example.

The substituted cyclopentadiene compound (9) is as follows:

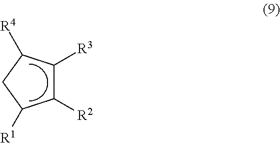

wherein R¹, R², R³ and R⁴ are as defined above, and

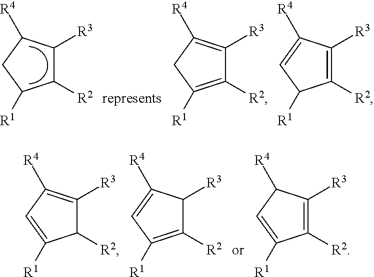

Examples of the substituted cyclopentadiene compound (9) include the following compounds:

cyclopentadiene, methylcyclopentadiene, 1,2-dimethylcyclopentadiene, 1,3-dimethylcyclopentadiene, 1,2,3-trimethylcyclopentadiene, 1,2,4-trimethylcyclopentadiene, 1,2,3,4-tetramethylcyclopentadiene, ethylcyclopentadiene, 1,2-diethylcyclopentadiene, 1,3-diethylcyclopentadiene, 1,2,3-triethylcyclopentadiene, 1,2,4-triethylcyclopentadiene, 1,2,3,4-tetraethylcyclopentadiene, n-propylcyclopentadiene, isopropylcyclopentadiene, n-butylcyclopentadiene, sec-butylcyclopentadiene, tert-butylcyclopentadiene, n-pentylcyclopentadiene, neopentylcyclopentadiene, n-hexylcyclopentadiene, n-octylcyclopentadiene, phenylcyclopentadiene, naphthylcyclopentadiene, trimethylsilylcyclopentadiene, triethylsilylcyclopentadiene, tert-butyldimethylsilylcyclopentadiene, indene, 2-methylindene, tetrahydroindene, 2-methyltetrahydroindene, 3-methyltetrahydroindene, 2,3-dimethyltetrahydroindene, 2-ethyltetrahydroindene, 2-n-propyltetrahydroindene, 2-isopropyltetrahydroindene, 2-n-butyltetrahydroindene, 2-sec-butyltetrahydroindene, 2-tert-butyltetrahydroindene, 2-n-pentyltetrahydroindene, 2-neopentyltetrahydroindene, 2-amyltetrahydroindene, 2-n-hexyltetrahydroindene, 2-cyclohexyltetrahydroindene, 2-n-octyltetrahydroindene, 2-n-decyltetrahydroindene, 2-phenyltetrahydroindene, 2-benzyltetrahydroindene, 2-naphthyltetrahydroindene, 2-methoxytetrahydroindene, 2-phenoxytetrahydroindene, 2-benzyloxytetrahydroindene, 2-dimethylaminotetrahydroindene, 2-trimethylsilyltetrahydroindene, fluorene and octahydrofluorene.

The substituted cyclopentadiene compounds (9) exemplified above may have isomers differing in the double bond position of each cyclopentadiene ring. A mixture of these isomers may be used.

The halogenated silyl compound (10-3) is as follows:

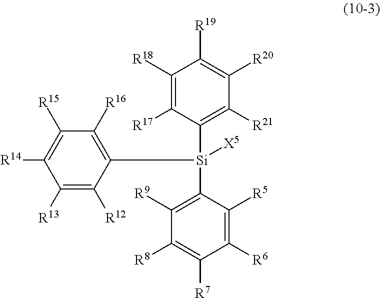

wherein R⁵, R⁶, R⁷, R⁸, R⁹, R¹², R¹³, R¹⁴, R¹⁵, R¹⁶, R¹⁷, R¹⁸, R¹⁹, R²⁰ and R²¹ are as defined above, and X⁵ is a halogen atom.

Examples of the halogenated silyl compound (10-1) include the following halogenated silyl compounds:

chlorodimethylphenylsilane, chlorodiethylphenylsilane, chlorophenyldi(n-propyl)silane, chlorodiisopropylphenylsilane, di(n-butyl)chlorophenylsilane, di(isobutyl)chlorophenylsilane, di(sec-butyl)chlorophenylsilane, di(tert-butyl)chlorophenylsilane, chloroethylmethylphenylsilane, chloromethylphenyl(n-propyl)silane, chloromethylphenyl(isopropyl)silane, n-butylchloromethylphenylsilane, isobutylchloromethylphenylsilane, sec-butylchloromethylphenylsilane, tert-butylchloromethylphenylsilane, chlorocyclohexylmethylphenylsilane, chloromethyl(n-octadecyl)phenylsilane,

chlorodimethyl(3,5-dimethylphenyl)silane, chlorodiethyl(3,5-dimethylphenyl)silane, chloro(3,5-dimethylphenyl)di(n-propyl)silane, chlorodiisopropyl(3,5-dimethylphenyl)silane, di(n-butyl)chloro(3,5-dimethylphenyl)silane, di(isobutyl)chloro(3,5-dimethylphenyl)silane, di(sec-butyl)chloro(3,5-dimethylphenyl)silane, di(tert-butyl)chloro(3,5-dimethylphenyl)silane, chloroethylmethyl(3,5-dimethylphenyl)silane, chloromethyl(3,5-dimethylphenyl)(n-propyl)silane, chloromethyl(3,5-dimethylphenyl)(isopropyl)silane, n-butylchloromethyl(3,5-dimethylphenyl)silane, isobutylchloromethyl(3,5-dimethylphenyl)silane, sec-butylchloromethyl(3,5-dimethylphenyl)silane, tert-butylchloromethyl(3,5-dimethylphenyl)silane, chlorocyclohexylmethyl(3,5-dimethylphenyl)silane and chloromethyl(n-octadecyl)(3,5-dimethylphenyl)silane.

Compounds obtained by substituting “fluoro”, “bromo” or “iodo” for “chloro” in these compounds exemplified above are also included therein.

Examples of the halogenated silyl compound (10-2) include the following halogenated silyl compounds:

chloromethyldiphenylsilane, chloroethyldiphenylsilane, chloro-n-propyldiphenylsilane, chloroisopropyldiphenylsilane, n-butylchlorodiphenylsilane, isobutylchlorodiphenylsilane, sec-butylchlorodiphenylsilane, tert-butylchlorodiphenylsilane, chlorocyclohexyldiphenylsilane, chloro-n-octadecyldiphenylsilane, chloromethylphenyl(2-methylphenyl)silane, chloromethylphenyl(3-methylphenyl)silane, chloromethylphenyl(4-methylphenyl)silane, chloromethylphenyl(2,3-dimethylphenyl)silane, chloromethylphenyl(2,4-dimethylphenyl)silane, chloromethylphenyl(2,5-dimethylphenyl)silane, chloromethylphenyl(2,6-dimethylphenyl)silane, chloromethylphenyl(3,5-dimethylphenyl)silane, chloromethylphenyl(3,4,5-trimethylphenyl)silane,

chloroethylphenyl(3,5-dimethylphenyl)silane, chloro-n-propylphenyl(3,5-dimethylphenyl)silane, chloroisopropylphenyl(3,5-dimethylphenyl)silane, n-butylchlorophenyl(3,5-dimethylphertyl)silane, isobutylchlorophenyl(3,5-dimethylphenyl)silane, sec-butylchlorophenyl(3,5-dimethylphenyl)silane, tert-butylchlorophenyl(3,5-dimethylphenyl)silane, chlorocyclohexylphenyl(3,5-dimethylphenyl)silane, chloro-n-octadecylphenyl(3,5-dimethylphenyl)silane, chloromethyl(2-methylphenyl)(3,5-dimethylphenyl)silane, chloromethyl(3-methylphenyl)(3,5-dimethylphenyl)silane, chloromethyl(4-methylphenyl)(3,5-dimethylphenyl)silane, chloromethyl(2,3-dimethylphenyl)(3,5-dimethylphenyl)silane, chloromethyl(2,4-dimethylphenyl)(3,5-dimethylphenyl)silane, chloromethyl(2,5-dimethylphenyl)(3,5-dimethylphenyl)silane, chloromethylphenyl(2,6-dimethylphenyl)(3,5-dimethylphenyl)silane, chloromethylbis(3,5-dimethylphenyl)silane and chloromethyl(3,5-dimethylphenyl)(3,4,5-trimethylphenyl)silane.

Compounds obtained by substituting “fluoro”, “bromo” or “iodo” for “chloro” in these compounds exemplified above are also included therein.

Examples of the halogenated silyl compound (10-3) include the following halogenated silyl compounds:

chlorotriphenylsilane, chlorophenyldi(2-methylphenyl)silane, chlorophenyldi(3-methylphenyl)silane, chlorophenyldi(4-methylphenyl)silane, chlorophenylbis(2,3-dimethylphenyl)silane, chlorophenylbis(2,4-dimethylphenyl)silane, chlorophenylbis(2,5-dimethylphenyl)silane, chlorophenylbis(2,6-dimethylphenyl)silane, chlorophenylbis(3,5-dimethylphenyl)silane, chlorophenylbis(3,4,5-trimethylphenyl)silane,

chlorodiphenyl(2-methylphenyl)silane, chlorodiphenyl(3-methylphenyl)silane, chlorodiphenyl(4-methylphenyl)silane, chlorodiphenyl(2,3-dimethylphenyl)silane, chlorodiphenyl(2,4-dimethylphenyl)silane, chlorodiphenyl(2,5-dimethylphenyl)silane, chlorodiphenyl(2,6-dimethylphenyl)silane, chlorodiphenyl(3,5-dimethylphenyl)silane, chlorodiphenyl(3,4,5-trimethylphenyl)silane,

chlorophenyl(2-methylphenyl)(3,5-dimethylphenyl)silane, chlorophenyl(3-methylphenyl)(3,5-dimethylphenyl)silane, chlorophenyl(4-methylphenyl)(3,5-dimethylphenyl)silane, chlorophenyl(2,3-dimethylphenyl)(3,5-dimethylphenyl)silane, chlorophenyl(2,4-dimethylphenyl)(3,5-dimethylphenyl)silane, chlorophenyl(2,5-dimethylphenyl)(3,5-dimethylphenyl)silane, chlorophenyl(2,6-dimethylphenyl)(3,5-dimethylphenyl)silane, chlorophenyl(3,5-dimethylphenyl)(3,4,5-trimethylphenyl)silane,

chlorodi(2-methylphenyl)(3,5-dimethylphenyl)silane, chlorodi(3-methylphenyl)(3,5-dimethylphenyl)silane, chlorodi(4-methylphenyl)(3,5-dimethylphenyl)silane, chlorobis(2,3-dimethylphenyl)(3,5-dimethylphenyl)silane, chlorobis(2,4-dimethylphenyl)(3,5-dimethylphenyl)silane, chlorobis(2,5-dimethylphenyl)(3,5-dimethylphenyl)silane, chlorobis(2,6-dimethylphenyl)(3,5-dimethylphenyl)silane, chlorotris(3,5-dimethylphenyl)silane and chloro(3,5-dimethylphenyl)bis(3,4,5-dimethylphenyl)silane.

Compounds obtained by substituting “fluoro”, “bromo” or “iodo” for “chloro” in these compounds exemplified above are also included therein.

Examples of the base reacted with the substituted cyclopentadiene compound (9) include: alkali metal hydride such as lithium hydride, sodium hydride and potassium hydride; alkaline earth metal hydride such as calcium hydride; and organic alkali metal compounds typified by organic lithium compounds such as methyllithium, ethyllithium, n-butyllithium, sec-butyllithium, tert-butyllithium, lithiumtrimethylsilyl acetylide, lithium acetylide, trimethylsilylmethyllithium, vinyllithium, phenyllithium and allyllithium. The amount thereof used is usually in the range of 0.5- to 3-fold by mol, preferably 0.9- to 2-fold by mol, with respect to the substituted cyclopentadiene compound (9). A usual commercially available mineral oil-containing product can be used directly as sodium hydride or potassium hydride. Of course, the mineral oil may be removed, for use, by washing with a hydrocarbyl solvent such as hexane.

In the step of reacting the substituted cyclopentadiene compound (9) with a base, an amine compound may be used. Examples of such an amine compound include: primary anilines such as aniline, chloroaniline, bromoaniline, fluoroaniline, dichloroaniline, dibromoaniline, difluoroaniline, trichloroaniline, tribromoaniline, trifluoroaniline, tetrachloroaniline, tetrabromoaniline, tetrafluoroaniline, pentachloroaniline, pentafluoroaniline, nitroaniline, dinitroaniline, hydroxyaniline, phenylenediamine, anisidine, dimethoxyaniline, trimethoxyaniline, ethoxyaniline, diethoxyaniline, triethoxyaniline, n-propoxyaniline, isopropoxyaniline, n-butoxyaniline, sec-butoxyaniline, isobutoxyaniline, t-butoxyaniline, phenoxyaniline, methylaniline, ethylaniline, n-propylaniline, isopropylaniline, n-butylaniline, sec-butylaniline, isobutylaniline, t-butylaniline, dimethylaniline, diethylaniline, di-n-propylaniline, diisopropylaniline, di-n-butylaniline, di-sec-butylaniline, diisobutylaniline, di-t-butylaniline, trimethylaniline, triethylaniline, diisopropylaniline, phenylaniline, benzylaniline, aminobenzoic acid, methyl aminobenzoate, ethyl aminobenzoate, n-propyl aminobenzoate, isopropyl aminobenzoate, n-butyl aminobenzoate, isobutyl aminobenzoate, sec-butyl aminobenzoate and t-butyl aminobenzoate, and other primary amines including naphthylamine, naphthylmethylamine, benzylamine, propylamine, butylamine, pentylamine, hexylamine, cyclohexylamine, heptylamine, octylamine, 2-aminopyridine, 3-aminopyridine and 4-aminopyridine;

secondary amines such as N-methylaniline, N-ethylaniline, diphenylamine, N-methylchloroaniline, N-methylbromoaniline, N-methylfluoroaniline, N-methylanisidine, N-methylmethylaniline, N-methylethylaniline, N-methyl-n-propylaniline, N-methylisopropylaniline, diethylamine, dipropylamine, diisopropylamine, dipentylamine, dihexylamine, dicyclohexylamine, diheptylamine, dioctylamine, morpholine, piperidine, 2,2,6,6-tetramethylpiperidine, pyrrolidine, 2-methylaminopyridine, 3-methylaminopyridine and 4-methylaminopyridine; and

tertiary amines such as N,N-dimethylaniline, N,N-dimethylchloroaniline, N,N-dimethylbromoaniline, N,N-dimethylfluoroaniline, N,N-dimethylanisidine, N-methylmethylaniline, N,N-dimethylethylaniline, N,N-dimethyl-n-propylaniline, N,N-dimethylisopropylaniline, 1,4-diazabicyclo[2.2.2]octane, 1,5-diazabicyclo[4.3.0]non-5-ene, 1,8-diazabicyclo[5.4.0]undec-7-ene, 2-di methylaminopyridine, 3-dimethylaminopyridine, 4-dimethylaminopyridine, trimethylamine, triethylamine, tri-n-propylamine, tri-n-butylamine, diisopropylethylamine, tri-n-octylamine, tri-n-decylamine and triphenylamine. Preferably primary or secondary amines, more preferably primary amines are used.

The amount of such an amine compound used is usually in the range of 0.001- to 2-fold by mol, preferably 0.01- to 0.5-fold by mol, with respect to the base. The reaction is usually performed in a solvent inert to the reaction. Examples of such a solvent include aprotic solvents such as: aromatic hydrocarbyl solvents such as benzene, toluene and xylene; aliphatic hydrocarbyl solvents such as pentane, hexane, heptane, octane and cyclohexane; ether solvents such as diethyl ether, methyl t-butyl ether, tetrahydrofuran and 1,4-dioxane; amide solvents such as hexamethylphosphoric amide, dimethylformamide, dimethylacetamide and N-methylpyrrolidone; and halogen solvents such as chlorobenzene and dichlorobenzene. These solvents are used alone or as a mixture of two or more thereof, and the amount thereof used is usually in the range of 1- to 200-fold by weight, preferably 3- to 30-fold by weight, with respect to each cyclopentadiene compound.

For the reaction, for example, the substituted cyclopentadiene compound (9), the base and the amine compound may be mixed simultaneously in a solvent, or the base and the amine compound are mixed in advance and then the substituted cyclopentadiene compound (9) may be added to the mixture. The reaction temperature is not particularly limited, and a temperature region that eliminates the need of low temperature equipment is industrially preferable and is, for example, in the range of 0 to 70° C., preferably 10 to 60° C. This reaction efficiently produces a metal salt of the substituted cyclopentadiene compound (9). The metal salt of the substituted cyclopentadiene compound (9) thus obtained may be used directly in the form of the reaction mixture or may be taken from the reaction mixture. The former case usually suffices.

The reaction for obtaining the substituted cyclopentadiene compound (7-3) is usually performed in a solvent inert to the reaction. Examples of such a solvent include aprotic solvents such as: aromatic hydrocarbyl solvents such as benzene, toluene and xylene; aliphatic hydrocarbyl solvents such as pentane, hexane, heptane, octane and cyclohexane; ether solvents such as diethyl ether, methyl t-butyl ether, tetrahydrofuran and 1,4-dioxane; amide solvents such as hexamethylphosphoric amide, dimethylformamide, dimethylacetamide and N-methylpyrrolidone; and halogen solvents such as chlorobenzene and dichlorobenzene. These solvents are used alone or as a mixture of two or more thereof, and the amount thereof used is usually in the range of 1- to 200-fold by weight, preferably 3- to 30-fold by weight, with respect to the substituted cyclopentadiene compound (9). This reaction is usually performed, for example, by mixing the base, the amine compound and the substituted cyclopentadiene compound (9) in a solvent and then adding the halogenated silyl compound (10-3) to the mixture. However, even when a method is adopted in which these components are mixed simultaneously, the substituted cyclopentadiene compound (7-3) of interest is produced. The reaction temperature is not particularly limited, and a temperature region that eliminates the need of low temperature equipment is industrially advantageous and is, for example, in the range of 0 to 70° C., preferably 10 to 60° C.

The amount of the substituted cyclopentadiene compound (9) used is usually in the range of 0.5- to 5-fold by mol, preferably 0.8- to 3-fold by mol, with respect to the halogenated silyl compound (10-3).

After completion of the reaction, water, an aqueous sodium bicarbonate solution, an aqueous sodium carbonate solution, an aqueous ammonium chloride solution or an aqueous solution of hydrochloric acid or the like is added to the obtained reaction mixture. Then, organic and aqueous phases are separated to obtain a solution of the substituted cyclopentadiene compound (7-3) as the organic phase. When a water-compatible solvent is used in the reaction or when the amount of the solvent used in the reaction is too small to easily separate organic and aqueous phases, a water-insoluble organic solvent such as toluene, ethyl acetate or chlorobenzene may be added to the reaction mixture as appropriate, followed by separation into organic and aqueous phases. The obtained organic phase is concentrated to obtain the substituted cyclopentadiene compound (7-3). The obtained substituted cyclopentadiene compound (8-1) may be purified, if necessary, by a method such as distillation and column chromatography treatment.

The substituted cyclopentadiene compounds (7-4) and (7-5) can be produced by, for example, a method described in J. Organomet. Chem. 1999, 592, 84-94.

The substituted cyclopentadiene compound (7-6) can be produced by, for example, a method described in J. Organomet. Chem. 1995, 485, 173-178.

### EXAMPLES

The present invention will be described by way of Reference Examples, Examples and Comparative Examples.

### <Production of Transition Metal Complex>

Physical properties were measured by the following methods.

(1) Proton nuclear magnetic resonance spectrum (¹H-NMR)

Apparatus: EX270 manufactured by JEOL Ltd.
Sample cell: Tube (5 mm in diameter)
Measurement solvent: CDCl₃ or CD₂Cl₂
Sample concentration: 10 mg/0.5 mL (CDCl₃ or CD₂Cl₂)
Measurement temperature: Room temperature (about 25° C.)
Measurement parameter: Probe (5 mm in diameter), EXMOD NON, OBNUC ¹H, accumulated number 16 times or more
Repeat time: ACQTM 6 seconds, PD 1 second
Internal standard: CDCl₃ (7.26 ppm) or CD₂Cl₂ (5.32 ppm)

(2) Carbon nuclear magnetic resonance spectrum (¹³C-NMR)

Apparatus: EX270 manufactured by JEOL Ltd.
Sample cell: Tube (5 mm in diameter)
Measurement solvent: CDCl₃ or CD₂Cl₂
Sample concentration: 30 mg/0.5 mL (CDCl₃ or CD₂Cl₂)
Measurement temperature: Room temperature (about 25° C.)
Measurement parameter: Probe (5 mm in diameter), EXMOD BCM, OBNUC ¹³C, accumulated number 256 times or more
Repeat time: ACQTM 1.79 seconds, PD 1.21 seconds
Internal standard: CDCl₃ (77.0 ppm) or CD₂Cl₂ (53.8 ppm)

(3) Mass spectrum

[Electron ionization mass spectrometry (EI-MS)]
Apparatus: JMS-T100GC manufactured by 0.1EOL Ltd.
Ionization voltage: 70 eV
Ion source temperature: 230° C.
Acceleration voltage: 7 kV

### MASS RANGE: m/z 35-800

### Reference Example 1

### Synthesis of 1-tris(3,5-dimethylphenyl)silyl-2,3,4,5-tetramethylcyclopentadiene

Under a nitrogen atmosphere, sodium hydride (0.49 g, 20.45 mmol in terms of sodium hydride) dispersed in mineral oil and tetrahydrofuran (23 mL) were mixed. This mixture was heated 50° C. and aniline (0.13 g, 1.36 mmol) was added and stirred at 50° C. for one hour. To this, a solution dissolving 1,2,3,4-tetramethylcyclopenta-1,3-diene (1.83 g, 15.00 mmol) in tetrahydrofuran (6 mL) was added dropwise and stirred at 50° C. for 3.5 hours. This was cooled to 0° C. To this solution, a solution dissolving chlorotris(3,5-dimethylphenyl)silane (5.17 g, 13.64 mmol) in toluene (6 mL) was added dropwise and stirred at room temperature for 3 hours, and thereafter, stirred at 50° C. for 22 hours. The resultant mixture was added dropwise at 0° C. to a 10% aqueous sodium carbonate solution (40 mL). Toluene (50 mL) was added to separate an organic phase, and the organic phase was washed with water (50 mL) twice and further washed with saturated saline (50 mL). The organic phase was dried over sodium sulfate and then filtrated. The solvent was removed under reduced pressure. After purification was performed by silica gel column chromatography, the resultant solid substance, to which hexane at 50° C. was added, was filtrated to remove insoluble matter. The solvent was removed from the filtrate under reduced pressure. The resultant solid substance was washed with a small amount of hexane and then dried under reduced pressure to obtain 1-tris(3,5-dimethylphenyl)silyl-2,3,4,5-tetramethylcyclopentadiene (1.49 g, yield 23.4%).

¹H-NMR (CDCl₃, δ ppm): 1.54 (s, 6H), 1.60 (s, 6H), 2.27 (s, 18H), 3.73 (s, 1H), 6.98 (s, 3H), 7.17 (s, 6H)

Mass Spec (EI-MS, m/z): 464 (M⁺)

### Reference Example 2

### Synthesis of [1-tris(3,5-dimethylphenyl)silyl-2,3,4,5-tetramethylcyclopentadienyl]titanium trichloride (hereinafter, abbreviated to a “complex 1”)

Under a nitrogen atmosphere, to a toluene solution (20 mL) of 1-tris(3,5-dimethylphenyl)silyl-2,3,4,5-tetramethylcyclopentadiene (0.93 g, 2.00 mmol) and triethylamine (1.01 g, 10.00 mmol), a 1.67 M hexane solution of n-butyllithium (1.32 mL, 2.20 mmol) was added dropwise at −78° C. After the mixture was gradually warmed to room temperature, the mixture was stirred at room temperature for 5 hours. The resultant mixture was cooled to −78° C. and a solution dissolving titanium tetrachloride (0.42 g, 2.20 mmol) in toluene (2 mL) was added dropwise at the same temperature. After the mixture was gradually warmed to room temperature, the mixture was stirred at room temperature overnight. After completion of the reaction, the solvent was removed under reduced pressure. Thereafter, the residue, to which heptane was added, was filtrated to remove insolubles. The solvent was removed from the filtrate under reduced pressure to obtain a mixture of complex 1 and 2,2′-bis{tris(3,5-dimethylphenyl)silyl}-1,1′3,3′,4,4′,5,5′-octamethyl-5,5′-bi-1,3-cyclopentadienyl as an orange solid. Furthermore, the resultant mixture, to which diethyl ether was added, was filtrated to remove insolubles. The solvent was removed from the filtrate under reduced pressure. Pentane was added and cooling to −20° C. was performed. The resultant solid substance was filtrated, washed with a small amount of pentane, and then dried under reduced pressure to obtain complex 1 (0.03 g, yield 2.7%) as an orange solid.

¹H-NMR (CDCl₃, δ ppm): 2.03 (s, 614), 127 (s, 18H), 2.36 (s, 6H), 7.06 (s, 3H), 7.20 (s, 6H)

¹³C-NMR (CDCl₃, δ ppm): 14.52, 17.83, 21.41, 131.63, 132.93, 134.60, 137.03, 142.26, 146.34

Mass Spec (EI-MS, m/z): 616 (M⁺)

### Reference Example 3

### Synthesis of [1-tris(3,5-dimethylphenyl)silyl-2,3,4,5-tetramethylcyclopentadienyl]trimethyltitanium (hereinafter, abbreviated to a “complex 2”)

Under a nitrogen atmosphere, to a diethyl ether solution (5 mL) of complex 1 (87.0 mg, 0.14 mmol), a 3.00 M tetrahydrofuran solution of methylmagnesium chloride (0.23 mL, 0.70 mmol) was added dropwise at −20° C. The mixture was gradually warmed to room temperature while stirring. After completion of the reaction, the solvent was removed under reduced pressure. Thereafter, the residue, to which hexane was added, was filtrated to remove insolubles. The solvent was removed from the filtrate under reduced pressure. The resultant solid substance was dried under reduced pressure to obtain complex 2 (57.3 mg, yield 73.1%) as a light yellow solid.

¹H-NMR(CD₂Cl₂ δ ppm): 0.95 (s, 9H), 1.60 (s, 6H), 1.95 (s, 6H), 2.26 (s, 18H), 7.02 (s, 3H), 7.11 (s, 6H)

¹³C-NMR (CD₂Cl₂, δ ppm): 12.47, 15.53, 21.47, 63.32, 117.74, 129.08, 130.62, 131.25, 134.77, 135.68, 137.15

### <Production of 1-Hexene>

### (1) Trimerization Activity

Analysis was conducted using gas chromatography (Shimadzu GC-2010, DB-1 column).

### (2) Synthesis of Known Transition Metal Complex

[1-(1-Methyl-1-(3,5-dimethylphenyl)ethyl)-3-trimethylsitylcyclopentadienyl]titanium trichloride (hereinafter, referred to as “complex 3”) was synthesized in accordance with a method known in the art (Organometallics 2002, 21, 5122-5135).

### Example 1

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 80° C. Thereafter, ethylene was introduced so that the partial pressure of ethylene might become 0.5 MPa, and the system was stabilized. Aside from this, complex 1 was dissolved in a toluene solution of TIBA prepared at 10 mmol/L to prepare a solution having the complex concentration of 1 mmol/L. 1 mL of this toluene solution was added to the autoclave and N,N-dimethylanilinium tetrakis(pentafluorophenyl)borate (AB) (4.1 μmol) was then added. A trimerization reaction of ethylene was performed at 80° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated. 1-Hexene was obtained at an activity of 7.82×10⁶ g/mol complex/h and a polymer was obtained at an activity of 0.02×10⁶ g/mol complex/h. The 1-hexene/polymer ratio was 391.

### Example 2

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 80° C. Thereafter, ethylene was introduced so that the partial pressure of ethylene might become 0.5 MPa, and the system was stabilized. Aside from this, complex 2 was dissolved in a toluene solution of TIBA prepared at 10 mmol/L to prepare a solution having the complex concentration of 1 mmol/L. 1 mL of this toluene solution was added to the autoclave and N,N-dimethylanilinium tetrakis(pentafluorophenyl)borate (AB) (3.8 μmol) was then added. A trimerization reaction of ethylene was performed at 80° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated. 1-Hexene was obtained at an activity of 10.1×10⁶ g/mol complex/h and a polymer was obtained at an activity of 0.05×10⁶ g/mol complex/h. The 1-hexene/polymer ratio was 202.

### Example 3

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 40° C. Thereafter, ethylene was introduced so that the partial pressure of ethylene might become 0.5 MPa, and the system was stabilized. Aside from this, complex 3 was dissolved in a toluene solution of TIBA prepared at 10 mmol/L to prepare a solution having the complex concentration of 1 mmol/L. 1 mL of this toluene solution was added to the autoclave and 1.5 mL of a toluene solution of N,N-dimethylanilinium tetrakis(pentafluorophenyl)borate (AB) (2 μmol/mL) was then added. A trimerization reaction of ethylene was performed at 40° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated. 1-Hexene was obtained at an activity of 2.37×10⁶ g/mol complex/h and a polymer was obtained at an activity of 0.22×10⁶ g/mol complex/h. The 1-hexene/polymer ratio was 11.

### Example 4

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 80° C. Thereafter, ethylene was introduced so that the partial pressure of ethylene might become 0.5 MPa, and the system was stabilized. Aside from this, complex 1 was dissolved in a hexane solution of triethylaluminum prepared at 10 mmol/L to prepare a solution having the complex concentration of 1 mmol/L. 1 mL of this toluene solution was added to the autoclave and 1.5 mL of a toluene solution of N,N-dimethylanilinium tetrakis(pentafluorophenyl)borate (AB) (2 μmol/mL) was then added. A trimerization reaction of ethylene was performed at 80° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated. 1-Hexene was obtained at an activity of 6.76×10⁶ g/mol complex/h and a polymer was obtained at an activity of 0.02×10⁶ g/mol complex/h. The 1-hexene/polymer ratio was 338.

### Example 5

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 80° C. Thereafter, ethylene was introduced so that the partial pressure of ethylene might become 0.5 MPa, and the system was stabilized. Aside from this, complex 2 was dissolved in a hexane solution of tri-normal octylaluminum prepared at 10 mmol/L to prepare a solution having the complex concentration of 1 mmol/L. 1 mL of this toluene solution was added to the autoclave and 1.5 mL of a toluene solution of N,N-dimethylanilinium tetrakis(pentafluorophenyl)borate (AB) (2 μmol/mL) was then added. A trimerization reaction of ethylene was performed at 80° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated. 1-Hexene was obtained at an activity of 5.99×10⁶ g/mol complex/h and a polymer was obtained at an activity of 0.27×10⁶ g/mol complex/h. The 1-hexene/polymer ratio was 22.2.

### Comparative Example 1

An autoclave (0.4 liter) equipped with a stirrer was dried under reduced pressure and then purged with argon. Toluene (90 mL) and a hexane solution (0.43 mL) of triisobutylaluminum (TIBA) having a concentration of 0.93 mmol/mL were supplied and the interior temperature of the system was elevated to 40° C. Thereafter, ethylene was introduced so that the partial pressure of ethylene might become 0.5 MPa, and the system was stabilized. 1.0 mL of a toluene solution of complex 3 (1 μmol/mL) was added thereto and 1.5 mL of a toluene solution of N,N-dimethylanilinium tetrakis(pentafluorophenyl)borate (AB) (2 μmol/mL) was then added. A trimerization reaction of ethylene was performed at 40° C. for 30 minutes while continuously supplying ethylene gas so as to maintain the total pressure at a constant value. Ethanol (1.0 mL) was added to terminate the reaction. Thereafter, ethylene was purged and the content of the autoclave was decalcificated with ethanol-hydrochloric acid and filtrated, 1-Hexene was obtained at an activity of 1.56×10⁶ g/mol complex/h and a polymer was obtained at an activity of 0.31×10⁶ g/mol complex/h. The 1-hexene/polymer ratio was 5.

### INDUSTRIAL APPLICABILITY

Since the present invention provides a method for producing 1-hexene that is capable of reducing the amount of by-product polymers even under high temperature conditions when 1-hexene is produced through the trimerization reaction of ethylene, the present invention is highly valuable in various fields of industries, especially in the field of methods for producing 1-hexene.

1. A method for producing 1-hexene, comprising the following steps 1 and 2:
step 1: the step of preparing a catalytic component by bringing a transition metal complex represented by any one of formulae (1-1) to (1-3) into contact with the following compound (A) in the absence of ethylene; and
step 2: the step of trimerizing ethylene in the presence of a catalyst obtainable by bringing the catalytic component obtained in step 1 into contact with the following compound (B): 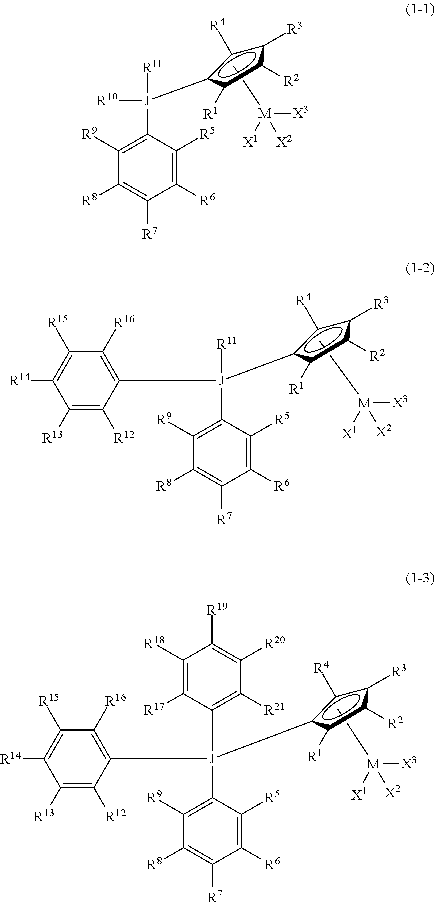
wherein
M represents a transition metal atom of Group 4 of the Periodic Table of the Elements; J represents a carbon atom or a silicon atom;
R¹, R², R³, R⁴, R⁵, R⁶, R⁷, R⁸, R⁹, R¹², R¹³, R¹⁴, R¹⁵, R¹⁶, R¹⁷, R¹⁸, R¹⁹, R²⁰, R²¹, X¹, X² and X³ each independently represent
a hydrogen atom, a halogen atom,
an alkyl group having 1 to 20 carbon atoms which may have a halogen atom as a substituent,
an alkoxy group having 1 to 20 carbon atoms which may have a halogen atom as a substituent,
an aryl group having 6 to 20 carbon atoms which may have a halogen atom as a substituent,
an aryloxy group having 6 to 20 carbon atoms which may have a halogen atom as a substituent,
an aralkyl group having 7 to 20 carbon atoms which may have a halogen atom as a substituent,
an aralkyloxy group having 7 to 20 carbon atoms which may have a halogen atom as a substituent,
a substituted silyl group represented by —Si(R²²)₃, wherein the three R²² groups each independently represent a hydrogen atom, a hydrocarbyl group or a halogenated hydrocarbyl group, and the total number of the carbon atoms in the three R²² groups is 1 to 20, or
a disubstituted amino group represented by —N(R²³)₂, wherein the two R²³ groups each independently represent a hydrocarbyl group or a halogenated hydrocarbyl group, and the total number of the carbon atoms in the two R²³ groups is 2 to 20, and
R¹⁹ and R¹¹ each independently represent
a hydrogen atom, a halogen atom,
an alkyl group having 1 to 20 carbon atoms which may have a halogen atom as a substituent,
an alkoxy group having 1 to 20 carbon atoms which may have a halogen atom as a substituent,
an aryloxy group having 6 to 20 carbon atoms which may have a halogen atom as a substituent,
an aralkyl group having 7 to 20 carbon atoms which may have a halogen atom as a substituent,
an aralkyloxy group having 7 to 20 carbon atoms which may have a halogen atom as a substituent,
a substituted silyl group represented by —Si(R²²)₃, wherein the three R²² groups each independently represent a hydrogen atom, a hydrocarbyl group or a halogenated hydrocarbyl group, and the total number of the carbon atoms in the three R²² groups is 1 to 20, or
a disubstituted amino group represented by —N(R²³)₂, wherein the two R²³ groups each independently represent a hydrocarbyl group or a halogenated hydrocarbyl group, and the total number of the carbon atoms in the two R²³ groups is 2 to 20;
of R¹, R², R³ and R⁴, two group bonded to two adjoining carbon atoms may be bonded to each other to form a ring together with the two carbon atoms to which the two groups are bonded, of R⁵, R⁶, R⁷, R⁸, R⁹, two group bonded to two adjoining carbon atoms may be bonded to each other to form a ring together with the two carbon atoms to which the two groups are bonded, of R¹², R¹³, R¹⁴, R¹⁵ and R¹⁶, two group bonded to two adjoining carbon atoms may be bonded to each other to form a ring together with the two carbon atoms to which the two groups are bonded, of R¹⁷, R¹⁸, R¹⁹, R²⁰ and R²¹, two group bonded to two adjoining carbon atoms may be bonded to each other to form a ring together with the two carbon atoms bonded to which the two groups are bonded, and R¹⁰ and R¹¹ may be bonded to each other to form a ring together with J to which they are bonded.
compound (A): an organic aluminum compound represented by formula
(E¹)ₐAl(G)₃₋ₐ,
wherein E¹ represents a hydrocarbyl group having 2 to 8 carbon atoms; G represents a hydrogen atom or a halogen atom; a represents an integer of 1 to 3; in the case where more than one E¹ groups exist, the E¹ groups may be the same as or different from each other; and in the case where more than one G groups exist, the G groups may be the same as or different from each other, and
compound (B): one or more boron compounds selected from the compound group consisting of the following compounds (B1), (B2) and (B3):
(B1): a boron compound represented by formula BQ¹Q²Q³,
(B2): a borate compound represented by formula T⁺(BQ⁴Q⁵Q⁶Q⁷)⁻, and
(B3): a borate compound represented by formula (L-H)⁺(BQ⁸Q⁹Q¹⁰Q¹¹)⁻, wherein
B represents a trivalent boron; Q¹, Q², Q³, Q⁴, Q⁵, Q⁶, Q⁷, Q⁸, Q⁹, Q¹⁰ and Q¹¹ are the same as or different from each other and each represent a halogen atom, a hydrocarbyl group having 1 to 20 carbon atoms which may have a halogen atom as a substituent, a hydrocarbylsilyl group having 1 to 20 carbon atoms which may have a halogen atom as a substituent, an alkoxy group having 1 to 20 carbon atoms which may have a halogen atom as a substituent or a dihydrocarbylamino group having 2 to 20 carbon atoms which may have a halogen atom as a substituent; T⁺ represents an inorganic or organic cation; and (L-H)⁺ represents a Broensted acid.

2. The method for producing 1-hexene according to claim 1, wherein X¹, X² and X³ each are a halogen atom.

3. The method for producing 1-hexene according to claim 1, wherein X¹, X² and X³ each are an alkyl group having 1 to 20 carbon atoms which may have a halogen atom as a substituent.

4. The method for producing 1-hexene according to claim 1, wherein M is a titanium atom.

5. The method for producing 1-hexene according to claim 1, wherein J is a silicon atom.
